SEQUENCE LISTING (726 sequences)

SEQ ID NO: 1 - DNA, 20 bp, Artificial
  Oligonucleotide primer
gaggtgcagctgttggagtc

SEQ ID NO: 2 - DNA, 69 bp, Artificial
  Oligonucleotide primer
gcccttcacggagtctgcgtamnntgtmnnmnnaccmnnmnnmnnaatmnntgagacccactccagccc

SEQ ID NO: 3 - DNA, 69 bp, Artificial
  Oligonucleotide primer
tabhtgagacccactccagcccgcccttcacggagtctgcgtaabhtgtabhabhaccabhabhabhaa

SEQ ID NO: 4 - DNA, 19 bp, Artificial
  Oligonucleotide primer
cgcagactccgtgaagggc

SEQ ID NO: 5 - DNA, 53 bp, Artificial
  Oligonucleotide primer
tccctggccccagtagtcaaamnnmnnmnnmnntttcgcacagtaatatacgg

SEQ ID NO: 6 - DNA, 53 bp, Artificial
  Oligonucleotide primer
tccctggccccagtagtcaaaabhabhabhabhtttcgcacagtaatatacgg

SEQ ID NO: 7 - DNA, 20 bp, Artificial
  Oligonucleotide primer
gacatccagatgacccagtc

SEQ ID NO: 8 - DNA, 51 bp, Artificial
  Oligonucleotide primer
atgggaccccactttgcaamnnggatgcmnnatagatcaggagcttagggg

SEQ ID NO: 9 - DNA, 51 bp, Artificial
  Oligonucleotide primer
atgggaccccactttgcaaabhggatgcabhatagatcaggagcttagggg

SEQ ID NO: 10 - DNA, 19 bp, Artificial
  Oligonucleotide primer
ttgcaaagtggggtcccat

SEQ ID NO: 11 - DNA, 60 bp, Artificial
  Oligonucleotide primer
cttggtcccttggccgaacgtmnnaggmnnmnnmnnmnnctgttgacagtagtaagttgc

SEQ ID NO: 12 - DNA, 60 bp, Artificial
  Oligonucleotide primer
cttggtcccttggccgaacgtabhaggabhabhabhabhctgttgacagtagtaagttgc

SEQ ID NO: 13 - DNA, 53 bp, Artificial
  Oligonucleotide primer
ctggagcctggcggacccamnncatmnnatamnngctaaaggtgaatccagag

SEQ ID NO: 14 - DNA, 53 bp, Artificial
  Oligonucleotide primer
ctggagcctggcggacccaabhcatabhataabhgctaaaggtgaatccagag

SEQ ID NO: 15 - DNA, 19 bp, Artificial
  Oligonucleotide primer
tgggtccgccaggctccag

SEQ ID NO: 16 - DNA, 53 bp, Artificial
  Oligonucleotide primer
ctggtttctgctgataccamnntaamnnmnnmnnaatgctctgacttgcccgg

SEQ ID NO: 17 - DNA, 53 bp, Artificial
  Oligonucleotide primer
ctggtttctgctgataccaabhtaaabhabhabhaatgctctgacttgcccgg

SEQ ID NO: 18 - DNA, 21 bp, Artificial
  Oligonucleotide primer
tggtatcagcagaaaccaggg

SEQ ID NO: 19 - DNA, 60 bp, Artificial
  Oligonucleotide primer
cttggtcccttggccgaacgtmnnaggggtactgtaactctgttgacagtagtaagttgc

SEQ ID NO: 20 - DNA, 60 bp, Artificial
  Oligonucleotide primer
cttggtcccttggccgaacgtabhaggggtactgtaactctgttgacagtagtaagttgc

SEQ ID NO: 21 - DNA, 53 bp, Artificial
  Oligonucleotide primer
gtcctcgcaactgcggcccagccggccatggccgaggtgcagctgttggagtc

SEQ ID NO: 22 - DNA, 56 bp, Artificial
  Oligonucleotide primer
gaaccgcctccaccgctcgagacggtgaccagggttccctggccccagtagtcaaa

SEQ ID NO: 23 - DNA, 71 bp, Artificial
  Oligonucleotide primer
agcggtggaggcggttcaggcggaggtggcagcggcggtggcgggtcgacggacatccagatgacccagtc

SEQ ID NO: 24 - DNA, 59 bp, Artificial
  Oligonucleotide primer
gagtcattctcgacttgcggccgcccgtttgatttccaccttggtcccttggccgaacg

SEQ ID NO: 25 - DNA, 23 bp, Artificial
  Oligonucleotide primer
gaggtbcarctgcaggastcygg

SEQ ID NO: 26 - protein, 240 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSGGGGSGGGGSGGGGSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 27 - DNA, 720 bp, Homo sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagttatggtgcttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctaatacgttcggccaagggaccaaggtggaaatcaaacgg

SEQ ID NO: 28 - protein, 5 aa, Homo sapiens
SYAMS

SEQ ID NO: 29 - protein, 17 aa, Homo sapiens
IIGSEGWPTIYADSVKG

SEQ ID NO: 30 - protein, 7 aa, Homo sapiens
GGSMFDY

SEQ ID NO: 31 - protein, 11 aa, Homo sapiens
RASQSISSYLN

SEQ ID NO: 32 - protein, 7 aa, Homo sapiens
RASSLQS

SEQ ID NO: 33 - protein, 9 aa, Homo sapiens
QQSSNTPYT

SEQ ID NO: 34 - protein, 5 aa, Homo sapiens
AYAMT

SEQ ID NO: 35 - protein, 17 aa, Homo sapiens
AISGSGGSTYYADSVKG

SEQ ID NO: 36 - protein, 7 aa, Homo sapiens
KASSFDY

SEQ ID NO: 37 - protein, 7 aa, Homo sapiens
AASSLQS

SEQ ID NO: 38 - protein, 9 aa, Homo sapiens
QQSYSTPST

SEQ ID NO: 39 - protein, 17 aa, Homo sapiens
LISPLGKDTSYADSVKG

SEQ ID NO: 40 - protein, 7 aa, Homo sapiens
RAGIFDY

SEQ ID NO: 41 - protein, 7 aa, Homo sapiens
HASRLQS

SEQ ID NO: 42 - protein, 9 aa, Homo sapiens
QQYRLRPLT

SEQ ID NO: 43 - protein, 17 aa, Homo sapiens
GIRRVGQATSYADSVKG

SEQ ID NO: 44 - protein, 7 aa, Homo sapiens
GGRLFDY

SEQ ID NO: 45 - protein, 7 aa, Homo sapiens
YASHLQS

SEQ ID NO: 46 - protein, 5 aa, Homo sapiens
KYRMF

SEQ ID NO: 47 - protein, 9 aa, Homo sapiens
QQYLLDPVT

SEQ ID NO: 48 - protein, 17 aa, Homo sapiens
AINTKGMTTDYADSVKG

SEQ ID NO: 49 - protein, 7 aa, Homo sapiens
GSQAFDY

SEQ ID NO: 50 - protein, 7 aa, Homo sapiens
QASFLQS

SEQ ID NO: 51 - protein, 9 aa, Homo sapiens
QQGYNKPRT

SEQ ID NO: 52 - protein, 5 aa, Homo sapiens
NYQMH

SEQ ID NO: 53 - protein, 7 aa, Homo sapiens
GTRRFDY

SEQ ID NO: 54 - protein, 9 aa, Homo sapiens
QQSYSTPVT

SEQ ID NO: 55 - protein, 17 aa, Homo sapiens
AISPKGRRTYYADSVKG

SEQ ID NO: 56 - protein, 7 aa, Homo sapiens
RDKLFDY

SEQ ID NO: 57 - protein, 7 aa, Homo sapiens
EASTLQS

SEQ ID NO: 58 - protein, 9 aa, Homo sapiens
QQEKMVPLT

SEQ ID NO: 59 - protein, 17 aa, Homo sapiens
RITPAGRRTTYADSVKG

SEQ ID NO: 60 - protein, 7 aa, Homo sapiens
PSPPFDY

SEQ ID NO: 61 - protein, 7 aa, Homo sapiens
HASILQS

SEQ ID NO: 62 - protein, 9 aa, Homo sapiens
QQGQHRPLT

SEQ ID NO: 63 - protein, 17 aa, Homo sapiens
RITPAGHRTYYADSVKG

SEQ ID NO: 64 - protein, 7 aa, Homo sapiens
QVSRFDY

SEQ ID NO: 65 - protein, 17 aa, Homo sapiens
TISPQGLRTTYADSVKG

SEQ ID NO: 66 - protein, 7 aa, Homo sapiens
GRPRFDY

SEQ ID NO: 67 - protein, 17 aa, Homo sapiens
TISPKGRSTTYADSVKG

SEQ ID NO: 68 - protein, 7 aa, Homo sapiens
TNRSFDY

SEQ ID NO: 69 - protein, 7 aa, Homo sapiens
RASRLQS

SEQ ID NO: 70 - protein, 9 aa, Homo sapiens
QQRAKKPPT

SEQ ID NO: 71 - protein, 7 aa, Homo sapiens
GRWPFDY

SEQ ID NO: 72 - protein, 11 aa, Homo sapiens
RASQSINENLS

SEQ ID NO: 73 - protein, 9 aa, Homo sapiens
QQSYSTPHT

SEQ ID NO: 74 - protein, 5 aa, Homo sapiens
RYRMH

SEQ ID NO: 75 - protein, 7 aa, Homo sapiens
NEPRFDY

SEQ ID NO: 76 - protein, 11 aa, Homo sapiens
RASQSIFMRLN

SEQ ID NO: 77 - protein, 9 aa, Homo sapiens
QQSYSTPST

SEQ ID NO: 78 - protein, 5 aa, Homo sapiens
RYRMG

SEQ ID NO: 79 - protein, 7 aa, Homo sapiens
GYRKFDY

SEQ ID NO: 80 - protein, 11 aa, Homo sapiens
RASQSISTLLN

SEQ ID NO: 81 - protein, 9 aa, Homo sapiens
QQSYSTPLT

SEQ ID NO: 82 - protein, 11 aa, Homo sapiens
RASQSIGPFLS

SEQ ID NO: 83 - protein, 9 aa, Homo sapiens
QQSYSTPPT

SEQ ID NO: 84 - protein, 11 aa, Homo sapiens
RASQSILRTLN

SEQ ID NO: 85 - protein, 9 aa, Homo sapiens
QQSYSTPGT

SEQ ID NO: 86 - protein, 17 aa, Homo sapiens
RIPARGTVTHYADSVKG

SEQ ID NO: 87 - protein, 7 aa, Homo sapiens
GGLRFDY

SEQ ID NO: 88 - protein, 7 aa, Homo sapiens
HASALQS

SEQ ID NO: 89 - protein, 9 aa, Homo sapiens
QQSYRKPTT

SEQ ID NO: 90 - protein, 17 aa, Homo sapiens
GISHTGSNTRYADSVKG

SEQ ID NO: 91 - protein, 7 aa, Homo sapiens
RHKGFDY

SEQ ID NO: 92 - protein, 7 aa, Homo sapiens
RASTLQS

SEQ ID NO: 93 - protein, 9 aa, Homo sapiens
QQGYRFPAT

SEQ ID NO: 94 - protein, 17 aa, Homo sapiens
RIAPEGGRTKYADSVKG

SEQ ID NO: 95 - protein, 7 aa, Homo sapiens
GRYWFDY

SEQ ID NO: 96 - protein, 7 aa, Homo sapiens
RASRLQS

SEQ ID NO: 97 - protein, 9 aa, Homo sapiens
QQSRNAPTT

SEQ ID NO: 98 - protein, 17 aa, Homo sapiens
TISYLGEKTRYADSVKG

SEQ ID NO: 99 - protein, 7 aa, Homo sapiens
SRRTFDY

SEQ ID NO: 100 - protein, 7 aa, Homo sapiens
KASTLQS

SEQ ID NO: 101 - protein, 9 aa, Homo sapiens
QQRSRPPAT

SEQ ID NO: 102 - protein, 5 aa, Homo sapiens
RYGMH

SEQ ID NO: 103 - protein, 7 aa, Homo sapiens
RGLGFDY

SEQ ID NO: 104 - protein, 9 aa, Homo sapiens
QQSYSTPLT

SEQ ID NO: 105 - protein, 5 aa, Homo sapiens
SYRMV

SEQ ID NO: 106 - protein, 7 aa, Homo sapiens
RGMAFDY

SEQ ID NO: 107 - protein, 11 aa, Homo sapiens
RASQSIHSRLS

SEQ ID NO: 108 - protein, 5 aa, Homo sapiens
KYNNH

SEQ ID NO: 109 - protein, 7 aa, Homo sapiens
ARWRFDY

SEQ ID NO: 110 - protein, 9 aa, Homo sapiens
QQSYSTPIT

SEQ ID NO: 111 - protein, 7 aa, Homo sapiens
TPRPFDY

SEQ ID NO: 112 - protein, 11 aa, Homo sapiens
RASQSIQMGLS

SEQ ID NO: 113 - protein, 9 aa, Homo sapiens
QQSYSTPNT

SEQ ID NO: 114 - protein, 11 aa, Homo sapiens
RASQSISENLL

SEQ ID NO: 115 - protein, 17 aa, Homo sapiens
TISPYGKQTRYADSVKG

SEQ ID NO: 116 - protein, 7 aa, Homo sapiens
KSQHFDY

SEQ ID NO: 117 - protein, 7 aa, Homo sapiens
AASRLQS

SEQ ID NO: 118 - protein, 9 aa, Homo sapiens
QQRGGGPPT

SEQ ID NO: 119 - protein, 17 aa, Homo sapiens
TITPRGSLTSYADSVKG

SEQ ID NO: 120 - protein, 7 aa, Homo sapiens
TAPPFDY

SEQ ID NO: 121 - protein, 7 aa, Homo sapiens
RASRLQS

SEQ ID NO: 122 - protein, 9 aa, Homo sapiens
QQSQRKPST

SEQ ID NO: 123 - protein, 17 aa, Homo sapiens
GISAYGTVTYYADSVKG

SEQ ID NO: 124 - protein, 7 aa, Homo sapiens
RRAGFDY

SEQ ID NO: 125 - protein, 9 aa, Homo sapiens
QQPRHMPQT

SEQ ID NO: 126 - protein, 17 aa, Homo sapiens
SITNSGLATAYADSVKG

SEQ ID NO: 127 - protein, 7 aa, Homo sapiens
RSFRFDY

SEQ ID NO: 128 - protein, 7 aa, Homo sapiens
HASRLQS

SEQ ID NO: 129 - protein, 9 aa, Homo sapiens
QQRHTNPPT

SEQ ID NO: 130 - protein, 17 aa, Homo sapiens
GITTRGQTTRYADSVKG

SEQ ID NO: 131 - protein, 7 aa, Homo sapiens
TYPKFDY

SEQ ID NO: 132 - protein, 7 aa, Homo sapiens
NASRLQS

SEQ ID NO: 133 - protein, 9 aa, Homo sapiens
QQSKLSPVT

SEQ ID NO: 134 - protein, 17 aa, Homo sapiens
TIPARGGHTKYADSVKG

SEQ ID NO: 135 - protein, 7 aa, Homo sapiens
SAKAFDY

SEQ ID NO: 136 - protein, 11 aa, Homo sapiens
RASRSISSYLN

SEQ ID NO: 137 - protein, 7 aa, Homo sapiens
QASNLQS

SEQ ID NO: 138 - protein, 9 aa, Homo sapiens
QQRSAGPLT

SEQ ID NO: 139 - protein, 5 aa, Homo sapiens
MYRMG

SEQ ID NO: 140 - protein, 7 aa, Homo sapiens
RTFRFDY

SEQ ID NO: 141 - protein, 11 aa, Homo sapiens
RASQSIRSRLS

SEQ ID NO: 142 - protein, 9 aa, Homo sapiens
QQSYSTPRT

SEQ ID NO: 143 - protein, 5 aa, Homo sapiens
SYAMT

SEQ ID NO: 144 - protein, 7 aa, Homo sapiens
KTGMFDY

SEQ ID NO: 145 - protein, 11 aa, Homo sapiens
RASQSIRTRLR

SEQ ID NO: 146 - protein, 17 aa, Homo sapiens
AINRRGSATRYADSVKG

SEQ ID NO: 147 - protein, 7 aa, Homo sapiens
YLHTFDY

SEQ ID NO: 148 - protein, 9 aa, Homo sapiens
QHPGLRPGT

SEQ ID NO: 149 - protein, 7 aa, Homo sapiens
AASALQS

SEQ ID NO: 150 - protein, 9 aa, Homo sapiens
QQSDLPPST

SEQ ID NO: 151 - protein, 5 aa, Homo sapiens
RYRMW

SEQ ID NO: 152 - protein, 7 aa, Homo sapiens
RPSTFDY

SEQ ID NO: 153 - protein, 11 aa, Homo sapiens
RASQSIAKNLS

SEQ ID NO: 154 - protein, 9 aa, Homo sapiens
QQSYSTPST

SEQ ID NO: 155 - protein, 11 aa, Homo sapiens
RASQSIKQRLH

SEQ ID NO: 156 - protein, 17 aa, Homo sapiens
SIAPAGRHTYYADSVKG

SEQ ID NO: 157 - protein, 7 aa, Homo sapiens
NIRIFDY

SEQ ID NO: 158 - protein, 7 aa, Homo sapiens
SASRLQS

SEQ ID NO: 159 - protein, 9 aa, Homo sapiens
QQRAGTPVT

SEQ ID NO: 160 - protein, 17 aa, Homo sapiens
GITMTGRTTKYADSVKG

SEQ ID NO: 161 - protein, 7 aa, Homo sapiens
NSMIFDY

SEQ ID NO: 162 - protein, 7 aa, Homo sapiens
QASRLQS

SEQ ID NO: 163 - protein, 9 aa, Homo sapiens
QQRVLRPPT

SEQ ID NO: 164 - protein, 5 aa, Homo sapiens
RYPMS

SEQ ID NO: 165 - protein, 7 aa, Homo sapiens
GFYAFDY

SEQ ID NO: 166 - protein, 11 aa, Homo sapiens
RASQSIVRVIT

SEQ ID NO: 167 - protein, 9 aa, Homo sapiens
QQSYSTPHT

SEQ ID NO: 168 - protein, 17 aa, Homo sapiens
TITASGPNTRYADSVKG

SEQ ID NO: 169 - protein, 7 aa, Homo sapiens
NHSTFDY

SEQ ID NO: 170 - protein, 7 aa, Homo sapiens
RASHLQS

SEQ ID NO: 171 - protein, 9 aa, Homo sapiens
QQNRTAPRT

SEQ ID NO: 172 - protein, 17 aa, Homo sapiens
TIYYAGSNTYYADSVKG

SEQ ID NO: 173 - protein, 7 aa, Homo sapiens
GYYTFDY

SEQ ID NO: 174 - protein, 7 aa, Homo sapiens
YASNLQS

SEQ ID NO: 175 - protein, 9 aa, Homo sapiens
QQSDTSPTT

SEQ ID NO: 176 - protein, 16 aa, Homo sapiens
MIYPGGYTKYADSVKG

SEQ ID NO: 177 - protein, 7 aa, Homo sapiens
NADLFDY

SEQ ID NO: 178 - protein, 7 aa, Homo sapiens
TASRLQS

SEQ ID NO: 179 - protein, 9 aa, Homo sapiens
QQMRRKPAT

SEQ ID NO: 180 - protein, 5 aa, Homo sapiens
LYNMV

SEQ ID NO: 181 - protein, 7 aa, Homo sapiens
EWSRFDY

SEQ ID NO: 182 - protein, 11 aa, Homo sapiens
RASQSISKSLI

SEQ ID NO: 183 - protein, 9 aa, Homo sapiens
QQSYSTPKT

SEQ ID NO: 184 - protein, 5 aa, Homo sapiens
GYYMS

SEQ ID NO: 185 - protein, 7 aa, Homo sapiens
THDSFDY

SEQ ID NO: 186 - protein, 11 aa, Homo sapiens
RASQSIDRYLN

SEQ ID NO: 187 - protein, 9 aa, Homo sapiens
QQSYSTPIT

SEQ ID NO: 188 - protein, 5 aa, Homo sapiens
RYQMV

SEQ ID NO: 189 - protein, 7 aa, Homo sapiens
HLSRFDY

SEQ ID NO: 190 - protein, 11 aa, Homo sapiens
RASQSIKYNLA

SEQ ID NO: 191 - protein, 9 aa, Homo sapiens
QQSYSTPRT

SEQ ID NO: 192 - protein, 17 aa, Homo sapiens
EILPRGHRTAYADSVKG

SEQ ID NO: 193 - protein, 7 aa, Homo sapiens
SGKHFDY

SEQ ID NO: 194 - protein, 7 aa, Homo sapiens
NASTLQS

SEQ ID NO: 195 - protein, 9 aa, Homo sapiens
QQRKRLPET

SEQ ID NO: 196 - protein, 5 aa, Homo sapiens
YYEML

SEQ ID NO: 197 - protein, 7 aa, Homo sapiens
PFMSFDY

SEQ ID NO: 198 - protein, 17 aa, Homo sapiens
SIGSSGYGTGYADSVKG

SEQ ID NO: 199 - protein, 7 aa, Homo sapiens
GYYSFDY

SEQ ID NO: 200 - protein, 7 aa, Homo sapiens
DASSLQS

SEQ ID NO: 201 - protein, 9 aa, Homo sapiens
QQSDSSPYT

SEQ ID NO: 202 - protein, 5 aa, Homo sapiens
DYDMS

SEQ ID NO: 203 - protein, 7 aa, Homo sapiens
DGAGFDY

SEQ ID NO: 204 - protein, 11 aa, Homo sapiens
RASQSIGSSLS

SEQ ID NO: 205 - protein, 9 aa, Homo sapiens
QQSYSTPNT

SEQ ID NO: 206 - protein, 17 aa, Homo sapiens
AISGLGKQTRYADSVKG

SEQ ID NO: 207 - protein, 7 aa, Homo sapiens
GYSRFDY

SEQ ID NO: 208 - protein, 7 aa, Homo sapiens
SASLLQS

SEQ ID NO: 209 - protein, 9 aa, Homo sapiens
QQLGTPPRT

SEQ ID NO: 210 - protein, 5 aa, Homo sapiens
RYEMS

SEQ ID NO: 211 - protein, 7 aa, Homo sapiens
SWTLFDY

SEQ ID NO: 212 - protein, 11 aa, Homo sapiens
RASQSIFTNLD

SEQ ID NO: 213 - protein, 9 aa, Homo sapiens
QQSYSTPPT

SEQ ID NO: 214 - protein, 11 aa, Homo sapiens
RASQSIGTLLR

SEQ ID NO: 215 - protein, 9 aa, Homo sapiens
QQSYSTPNT

SEQ ID NO: 216 - protein, 30 aa, Homo sapiens
QVQLVQSGAEVKKPGASVKVSCKASGYTFT

SEQ ID NO: 217 - protein, 4 aa, Homo sapiens
YYMH

SEQ ID NO: 218 - protein, 14 aa, Homo sapiens
WVRQAPGQGLEWMG

SEQ ID NO: 219 - protein, 4 aa, Homo sapiens
WINP

SEQ ID NO: 220 - protein, 13 aa, Homo sapiens
NSGGTNYAQKFQG

SEQ ID NO: 221 - protein, 32 aa, Homo sapiens
RVTMTRDTSISTAYMELSRLRSDDTAVYYCAR

SEQ ID NO: 222 - protein, 4 aa, Homo sapiens
YAMH

SEQ ID NO: 223 - protein, 14 aa, Homo sapiens
WVRQAPGQRLEWMG

SEQ ID NO: 224 - protein, 4 aa, Homo sapiens
WINA

SEQ ID NO: 225 - protein, 13 aa, Homo sapiens
GNGNTKYSQKFQG

SEQ ID NO: 226 - protein, 32 aa, Homo sapiens
RVTITRDTSASTAYMELSSLRSEDTAVYYCAR

SEQ ID NO: 227 - protein, 4 aa, Homo sapiens
YDIN

SEQ ID NO: 228 - protein, 14 aa, Homo sapiens
WVRQATGQGLEWMG

SEQ ID NO: 229 - protein, 4 aa, Homo sapiens
WMNP

SEQ ID NO: 230 - protein, 13 aa, Homo sapiens
NSGNTGYAQKFQG

SEQ ID NO: 231 - protein, 32 aa, Homo sapiens
RVTMTRNTSISTAYMELSSLRSEDTAVYYCAR

SEQ ID NO: 232 - protein, 4 aa, Homo sapiens
YGIS

SEQ ID NO: 233 - protein, 4 aa, Homo sapiens
WISA

SEQ ID NO: 234 - protein, 13 aa, Homo sapiens
YNGNTNYAQKLQG

SEQ ID NO: 235 - protein, 32 aa, Homo sapiens
RVTMTTDTSTSTAYMELRSLRSDDTAVYYCAR

SEQ ID NO: 236 - protein, 30 aa, Homo sapiens
QVQLVQSGAEVKKPGASVKVSCKVSGYTLT

SEQ ID NO: 237 - protein, 4 aa, Homo sapiens
LSMH

SEQ ID NO: 238 - protein, 14 aa, Homo sapiens
WVRQAPGKGLEWMG

SEQ ID NO: 239 - protein, 4 aa, Homo sapiens
GFDP

SEQ ID NO: 240 - protein, 13 aa, Homo sapiens
EDGETIYAQKFQG

SEQ ID NO: 241 - protein, 32 aa, Homo sapiens
RVTMTEDTSTDTAYMELSSLRSEDTAVYYCAT

SEQ ID NO: 242 - protein, 30 aa, Homo sapiens
QMQLVQSGAEVKKTGSSVKVSCKASGYTFT

SEQ ID NO: 243 - protein, 4 aa, Homo sapiens
RYLH

SEQ ID NO: 244 - protein, 14 aa, Homo sapiens
WVRQAPGQALEWMG

SEQ ID NO: 245 - protein, 4 aa, Homo sapiens
WITP

SEQ ID NO: 246 - protein, 13 aa, Homo sapiens
FNGNTNYAQKFQD

SEQ ID NO: 247 - protein, 32 aa, Homo sapiens
RVTITRDRSMSTAYMELSSLRSEDTAMYYCAR

SEQ ID NO: 248 - protein, 30 aa, Homo sapiens; identical to SEQ ID NO: 216

SEQ ID NO: 249 - protein, 4 aa, Homo sapiens
YYMH

SEQ ID NO: 250 - protein, 4 aa, Homo sapiens
IINP

SEQ ID NO: 251 - protein, 13 aa, Homo sapiens
SGGSTSYAQKFQG

SEQ ID NO: 252 - protein, 32 aa, Homo sapiens
RVTMTRDTSTSTVYMELSSLRSEDTAVYYCAR

SEQ ID NO: 253 - protein, 30 aa, Homo sapiens
QMQLVQSGPEVKKPGTSVKVSCKASGFTFT

SEQ ID NO: 254 - protein, 4 aa, Homo sapiens
SAVQ

SEQ ID NO: 255 - protein, 14 aa, Homo sapiens
WVRQARGQRLEWIG

SEQ ID NO: 256 - protein, 4 aa, Homo sapiens
WIVV

SEQ ID NO: 257 - protein, 13 aa, Homo sapiens
GSGNTNYAQKFQE

SEQ ID NO: 258 - protein, 32 aa, Homo sapiens
RVTITRDMSTSTAYMELSSLRSEDTAVYYCAA

SEQ ID NO: 259 - protein, 30 aa, Homo sapiens
QVQLVQSGAEVKKPGSSVKVSCKASGGTFS

SEQ ID NO: 260 - protein, 4 aa, Homo sapiens
YAIS

SEQ ID NO: 261 - protein, 14 aa, Homo sapiens
WVRQAPGQGLEWMG

SEQ ID NO: 262 - protein, 4 aa, Homo sapiens
GIIP

SEQ ID NO: 263 - protein, 13 aa, Homo sapiens
IFGTANYAQKFQG

SEQ ID NO: 264 - protein, 32 aa, Homo sapiens
RVTITADESTSTAYMELSSLRSEDTAVYYCAR

SEQ ID NO: 265 - protein, 32 aa, Homo sapiens
RVTITADKSTSTAYMELSSLRSEDTAVYYCAR

SEQ ID NO: 266 - protein, 30 aa, Homo sapiens
EVQLVQSGAEVKKPGATVKISCKVSGYTFT

SEQ ID NO: 267 - protein, 14 aa, Homo sapiens
WVQQAPGKGLEWMG

SEQ ID NO: 268 - protein, 4 aa, Homo sapiens
LVDP

SEQ ID NO: 269 - protein, 13 aa, Homo sapiens
EDGETIYAEKFQG

SEQ ID NO: 270 - protein, 32 aa, Homo sapiens
RVTITADTSTDTAYMELSSLRSEDTAVYYCAT

SEQ ID NO: 271 - protein, 30 aa, Homo sapiens
QITLKESGPTLVKPTQTLTLTCTFSGFSLS

SEQ ID NO: 272 - protein, 7 aa, Homo sapiens
TSGVGVG

SEQ ID NO: 273 - protein, 14 aa, Homo sapiens
WIRQPPGKALEWLA

SEQ ID NO: 274 - protein, 13 aa, Homo sapiens
WNDDKRYSPSLKS

SEQ ID NO: 275 - protein, 33 aa, Homo sapiens
RLTITKDTSKNQVVLTMTNMDPVDTATYYCAHR

SEQ ID NO: 276 - protein, 30 aa, Homo sapiens
QVTLKESGPVLVKPTETLTLTCTVSGFSLS

SEQ ID NO: 277 - protein, 7 aa, Homo sapiens
NARMGVS

SEQ ID NO: 278 - protein, 13 aa, Homo sapiens
SNDEKSYSTSLKS

SEQ ID NO: 279 - protein, 33 aa, Homo sapiens
RLTISKDTSKSQVVLTMTNMDPVDTATYYCARI

SEQ ID NO: 280 - protein, 30 aa, Homo sapiens
QVTLKESGPALVKPTQTLTLTCTFSGFSLS

SEQ ID NO: 281 - protein, 7 aa, Homo sapiens
TSGMRVS

SEQ ID NO: 282 - protein, 13 aa, Homo sapiens
WDDDKFYSTSLKT

SEQ ID NO: 283 - protein, 33 aa, Homo sapiens
RLTISKDTSKNQVVLTMTNMDPVDTATYYCARI

SEQ ID NO: 284 - protein, 30 aa, Homo sapiens
EVQLVESGGGLVQPGGSLRLSCAASGFTFS

SEQ ID NO: 285 - protein, 4 aa, Homo sapiens
YWMS

SEQ ID NO: 286 - protein, 14 aa, Homo sapiens
WVRQAPGKGLEWVA

SEQ ID NO: 287 - protein, 4 aa, Homo sapiens
NIKQ

SEQ ID NO: 288 - protein, 13 aa, Homo sapiens
DGSEKYYVDSVKG

SEQ ID NO: 289 - protein, 32 aa, Homo sapiens
RFTISRDNAKNSLYLQMNSLRAEDTAVYYCAR

SEQ ID NO: 290 - protein, 30 aa, Homo sapiens
EVQLVESGGGLVQPGRSLRLSCAASGFTFD

SEQ ID NO: 291 - protein, 14 aa, Homo sapiens
WVRQAPGKGLEWVS

SEQ ID NO: 292 - protein, 4 aa, Homo sapiens
GISW

SEQ ID NO: 293 - protein, 13 aa, Homo sapiens
NSGSIGYADSVKG

SEQ ID NO: 294 - protein, 33 aa, Homo sapiens
RFTISRDNAKNSLYLQMNSLRAEDTALYYCAKD

SEQ ID NO: 295 - protein, 30 aa, Homo sapiens
QVQLVESGGGLVKPGGSLRLSCAASGFTFS

SEQ ID NO: 296 - protein, 4 aa, Homo sapiens
YYMS

SEQ ID NO: 297 - protein, 14 aa, Homo sapiens
WIRQAPGKGLEWVS

SEQ ID NO: 298 - protein, 4 aa, Homo sapiens
YISS

SEQ ID NO: 299 - protein, 13 aa, Homo sapiens
SGSTIYYADSVKG

SEQ ID NO: 300 - protein, 30 aa, Homo sapiens; identical to SEQ ID NO: 284

SEQ ID NO: 301 - protein, 4 aa, Homo sapiens
YDMH

SEQ ID NO: 302 - protein, 14 aa, Homo sapiens
WVRQATGKGLEWVS

SEQ ID NO: 303 - protein, 13 aa, Homo sapiens
TAGDTYYPGSVKG

SEQ ID NO: 304 - protein, 32 aa, Homo sapiens
RFTISRENAKNSLYLQMNSLRAGDTAVYYCAR

SEQ ID NO: 305 - protein, 30 aa, Homo sapiens
EVQLVESGGGLVKPGGSLRLSCAASGFTFS

SEQ ID NO: 306 - protein, 4 aa, Homo sapiens
AWMS

SEQ ID NO: 307 - protein, 14 aa, Homo sapiens
WVRQAPGKGLEWVG

SEQ ID NO: 308 - protein, 19 aa, Homo sapiens
RIKSKTDGGTTDYAAPVKG

SEQ ID NO: 309 - protein, 32 aa, Homo sapiens
RFTISRDDSKNTLYLQMNSLKTEDTAVYYCTT

SEQ ID NO: 310 - protein, 30 aa, Homo sapiens
EVQLVESGGGVVRPGGSLRLSCAASGFTFD

SEQ ID NO: 311 - protein, 4 aa, Homo sapiens
YGMS

SEQ ID NO: 312 - protein, 4 aa, Homo sapiens
GINW

SEQ ID NO: 313 - protein, 13 aa, Homo sapiens
NGGSTGYADSVKG

SEQ ID NO: 314 - protein, 32 aa, Homo sapiens
RFTISRDNAKNSLYLQMNSLRAEDTALYHCAR

SEQ ID NO: 315 - protein, 30 aa, Homo sapiens; identical to SEQ ID NO: 305

SEQ ID NO: 316 - protein, 4 aa, Homo sapiens
YSMN

SEQ ID NO: 317 - protein, 4 aa, Homo sapiens
SISS

SEQ ID NO: 318 - protein, 13 aa, Homo sapiens
SSSYIYYADSVKG

SEQ ID NO: 319 - protein, 32 aa, Homo sapiens; identical to SEQ ID NO: 289

SEQ ID NO: 320 - protein, 30 aa, Homo sapiens
EVQLLESGGGLVQPGGSLRLSCAASGFTFS

SEQ ID NO: 321 - protein, 4 aa, Homo sapiens
YAMS

SEQ ID NO: 322 - protein, 4 aa, Homo sapiens
AISG

SEQ ID NO: 323 - protein, 13 aa, Homo sapiens
SGGSTYYADSVKG

SEQ ID NO: 324 - protein, 32 aa, Homo sapiens
RFTISRDNSKNTLYLQMNSLRAEDTAVYYCAK

SEQ ID NO: 325 - protein, 30 aa, Homo sapiens
QVQLVESGGGVVQPGRSLRLSCAASGFTFS

SEQ ID NO: 326 - protein, 4 aa, Homo sapiens
YGMH

SEQ ID NO: 327 - protein, 14 aa, Homo sapiens
WVRQAPGKGLEWVA

SEQ ID NO: 328 - protein, 4 aa, Homo sapiens
VISY

SEQ ID NO: 329 - protein, 13 aa, Homo sapiens
DGSNKYYADSVKG

SEQ ID NO: 330 - protein, 32 aa, Homo sapiens
RFTISRDNSKNTLYLQMNSLRAEDTAVYYCAR

SEQ ID NO: 331 - protein, 4 aa, Homo sapiens
VIWY

SEQ ID NO: 332 - protein, 30 aa, Homo sapiens
EVQLVESGGVVVQPGGSLRLSCAASGFTFD

SEQ ID NO: 333 - protein, 4 aa, Homo sapiens
YTMH

SEQ ID NO: 334 - protein, 4 aa, Homo sapiens
LISW

SEQ ID NO: 335 - protein, 13 aa, Homo sapiens
DGGSTYYADSVKG

SEQ ID NO: 336 - protein, 33 aa, Homo sapiens
RFTISRDNSKNSLYLQMNSLRTEDTALYYCAKD

SEQ ID NO: 337 - protein, 4 aa, Homo sapiens
YISS

SEQ ID NO: 338 - protein, 13 aa, Homo sapiens
SSSTIYYADSVKG

SEQ ID NO: 339 - protein, 32 aa, Homo sapiens
RFTISRDNAKNSLYLQMNSLRDEDTAVYYCAR

SEQ ID NO: 340 - protein, 30 aa, Homo sapiens
EVQLVESGGGLVQPGRSLRLSCTASGFTFG

SEQ ID NO: 341 - protein, 14 aa, Homo sapiens
WFRQAPGKGLEWVG

SEQ ID NO: 342 - protein, 19 aa, Homo sapiens
FIRSKAYGGTTEYTASVKG

SEQ ID NO: 343 - protein, 32 aa, Homo sapiens
RFTISRDGSKSIAYLQMNSLKTEDTAVYYCTR

SEQ ID NO: 344 - protein, 30 aa, Homo sapiens
EVQLVETGGGLIQPGGSLRLSCAASGFTVS

SEQ ID NO: 345 - protein, 4 aa, Homo sapiens
NYMS

SEQ ID NO: 346 - protein, 13 aa, Homo sapiens
SGGSTYYADSVKG

SEQ ID NO: 347 - protein, 14 aa, Homo sapiens
WVRQAPGKGLEYVS

SEQ ID NO: 348 - protein, 4 aa, Homo sapiens
AISS

SEQ ID NO: 349 - protein, 13 aa, Homo sapiens
NGGSTYYANSVKG

SEQ ID NO: 350 - protein, 32 aa, Homo sapiens
RFTISRDNSKNTLYLQMGSLRAEDMAVYYCAR

SEQ ID NO: 351 - protein, 30 aa, Homo sapiens
EVQLVESGGGLVQPGGSLRLSCAASGFTVS

SEQ ID NO: 352 - protein, 4 aa, Homo sapiens
HYMD

SEQ ID NO: 353 - protein, 14 aa, Homo sapiens
WVRQAPGKGLEWVG

SEQ ID NO: 354 - protein, 19 aa, Homo sapiens
RTRNKANSYTTEYAASVKG

SEQ ID NO: 355 - protein, 32 aa, Homo sapiens
RFTISRDDSKNSLYLQMNSLKTEDTAVYYCAR

SEQ ID NO: 356 - protein, 30 aa, Homo sapiens
EVQLVESGGGLVQPGGSLKLSCAASGFTFS

SEQ ID NO: 357 - protein, 4 aa, Homo sapiens
SAMH

SEQ ID NO: 358 - protein, 14 aa, Homo sapiens
WVRQASGKGLEWVG

SEQ ID NO: 359 - protein, 19 aa, Homo sapiens
RIRSKANSYATAYAASVKG

SEQ ID NO: 360 - protein, 32 aa, Homo sapiens
RFTISRDDSKNTAYLQMNSLKTEDTAVYYCTR

SEQ ID NO: 361 - protein, 4 aa, Homo sapiens
YWMH

SEQ ID NO: 362 - protein, 14 aa, Homo sapiens
WVRQAPGKGLVWVS

SEQ ID NO: 363 - protein, 4 aa, Homo sapiens
RINS

SEQ ID NO: 364 - protein, 13 aa, Homo sapiens
DGSSTSYADSVKG

SEQ ID NO: 365 - protein, 32 aa, Homo sapiens
RFTISRDNAKNTLYLQMNSLRAEDTAVYYCAR

SEQ ID NO: 366 - protein, 30 aa, Homo sapiens
EVQLVESRGVLVQPGGSLRLSCAASGFTVS

SEQ ID NO: 367 - protein, 4 aa, Homo sapiens
NEMS

SEQ ID NO: 368 - protein, 13 aa, Homo sapiens
SGGSTYYADSRKG

SEQ ID NO: 369 - protein, 32 aa, Homo sapiens
RFTISRDNSKNTLHLQMNSLRAEDTAVYYCKK

SEQ ID NO: 370 - protein, 30 aa, Homo sapiens
QVQLQESGPGLVKPSGTLSLTCAVSGGSIS

SEQ ID NO: 371 - protein, 4 aa, Homo sapiens
NWWS

SEQ ID NO: 372 - protein, 14 aa, Homo sapiens
WVRQPPGKGLEWIG

SEQ ID NO: 373 - protein, 13 aa, Homo sapiens
HSGSTNYNPSLKS

SEQ ID NO: 374 - protein, 32 aa, Homo sapiens
RVTISVDKSKNQFSLKLSSVTAADTAVYYCAR

SEQ ID NO: 375 - protein, 30 aa, Homo sapiens
QVQLQESGPGLVKPSDTLSLTCAVSGYSIS

SEQ ID NO: 376 - protein, 4 aa, Homo sapiens
NWWG

SEQ ID NO: 377 - protein, 14 aa, Homo sapiens
WIRQPPGKGLEWIG

SEQ ID NO: 378 - protein, 13 aa, Homo sapiens
YSGSTYYNPSLKS

SEQ ID NO: 379 - protein, 32 aa, Homo sapiens
RVTMSVDTSKNQFSLKLSSVTAVDTAVYYCAR

SEQ ID NO: 380 - protein, 30 aa, Homo sapiens
QVQLQESGPGLVKPSQTLSLTCTVSGGSIS

SEQ ID NO: 381 - protein, 7 aa, Homo sapiens
SGGYYWS

SEQ ID NO: 382 - protein, 14 aa, Homo sapiens
WIRQHPGKGLEWIG

SEQ ID NO: 383 - protein, 32 aa, Homo sapiens
RVTISVDTSKNQFSLKLSSVTAADTAVYYCAR

SEQ ID NO: 384 - protein, 30 aa, Homo sapiens
QLQLQESGSGLVKPSQTLSLTCAVSGGSIS

SEQ ID NO: 385 - protein, 7 aa, Homo sapiens
SGGYSWS

SEQ ID NO: 386 - protein, 13 aa, Homo sapiens
HSGSTYYNPSLKS

SEQ ID NO: 387 - protein, 32 aa, Homo sapiens
RVTISVDRSKNQFSLKLSSVTAADTAVYYCAR

SEQ ID NO: 388 - protein, 7 aa, Homo sapiens
SGDYYWS

SEQ ID NO: 389 - protein, 32 aa, Homo sapiens; identical to SEQ ID NO: 383

SEQ ID NO: 390 - protein, 30 aa, Homo sapiens
QVQLQQWGAGLLKPSETLSLTCAVYGGSFS

SEQ ID NO: 391 - protein, 4 aa, Homo sapiens
YYWS

SEQ ID NO: 392 - protein, 30 aa, Homo sapiens
QLQLQESGPGLVKPSETLSLTCTVSGGSIS

SEQ ID NO: 393 - protein, 7 aa, Homo sapiens
SSSYYWG

SEQ ID NO: 394 - protein, 13 aa, Homo sapiens
YSGSTNYNPSLKS

SEQ ID NO: 395 - protein, 30 aa, Homo sapiens
QVQLQESGPGLVKPSETLSLTCTVSGGSVS

SEQ ID NO: 396 - protein, 7 aa, Homo sapiens
SGSYYWS

SEQ ID NO: 397 - protein, 30 aa, Homo sapiens
QVQLQESGPGLVKPSETLSLTCAVSGYSIS

SEQ ID NO: 398 - protein, 4 aa, Homo sapiens
YYWG

SEQ ID NO: 399 - protein, 30 aa, Homo sapiens
EVQLVQSGAEVKKPGESLKISCKGSGYSFT

SEQ ID NO: 400 - protein, 4 aa, Homo sapiens
YWIG

SEQ ID NO: 401 - protein, 14 aa, Homo sapiens
WVRQMPGKGLEWMG

SEQ ID NO: 402 - protein, 4 aa, Homo sapiens
IIYP

SEQ ID NO: 403 - protein, 13 aa, Homo sapiens
GDSDTRYSPSFQG

SEQ ID NO: 404 - protein, 32 aa, Homo sapiens
QVTISADKSISTAYLQWSSLKASDTAMYYCAR

SEQ ID NO: 405 - protein, 30 aa, Homo sapiens
EVQLVQSGAEVKKPGESLRISCKGSGYSFT

SEQ ID NO: 406 - protein, 4 aa, Homo sapiens
YWIS

SEQ ID NO: 407 - protein, 4 aa, Homo sapiens
RIDP

SEQ ID NO: 408 - protein, 13 aa, Homo sapiens
SDSYTNYSPSFQG

SEQ ID NO: 409 - protein, 32 aa, Homo sapiens
HVTISADKSISTAYLQWSSLKASDTAMYYCAR

SEQ ID NO: 410 - protein, 30 aa, Homo sapiens
QVQLQQSGPGLVKPSQTLSLTCAISGDSVS

SEQ ID NO: 411 - protein, 7 aa, Homo sapiens
SNSAAWN

SEQ ID NO: 412 - protein, 14 aa, Homo sapiens
WIRQSPSRGLEWLG

SEQ ID NO: 413 - protein, 5 aa, Homo sapiens
RTYYR

SEQ ID NO: 414 - protein, 13 aa, Homo sapiens
SKWYNDYAVSVKS

SEQ ID NO: 415 - protein, 32 aa, Homo sapiens
RITINPDTSKNQFSLQLNSVTPEDTAVYYCAR

SEQ ID NO: 416 - protein, 30 aa, Homo sapiens
QVQLVQSGSELKKPGASVKVSCKASGYTFT

SEQ ID NO: 417 - protein, 4 aa, Homo sapiens
YAMN

SEQ ID NO: 418 - protein, 14 aa, Homo sapiens
WVRQAPGQGLEWMG

SEQ ID NO: 419 - protein, 4 aa, Homo sapiens
WINT

SEQ ID NO: 420 - protein, 13 aa, Homo sapiens
NTGNPTYAQGFTG

SEQ ID NO: 421 - protein, 32 aa, Homo sapiens
RFVFSLDTSVSTAYLQICSLKAEDTAVYYCAR

SEQ ID NO: 422 - protein, 23 aa, Homo sapiens
DIQMTQSPSSLSASVGDRVTITC

SEQ ID NO: 423 - protein, 8 aa, Homo sapiens
RASQSISS

SEQ ID NO: 424 - protein, 15 aa, Homo sapiens
WYQQKPGKAPKLLIY

SEQ ID NO: 425 - protein, 7 aa, Homo sapiens
AASSLQS

SEQ ID NO: 426 - protein, 32 aa, Homo sapiens
GVPSRFSGSGSGTDFTLTISSLQPEDFATYYC

SEQ ID NO: 427 - protein, 7 aa, Homo sapiens
QQSYSTP

SEQ ID NO: 428 - protein, 8 aa, Homo sapiens
QASQDISN

SEQ ID NO: 429 - protein, 7 aa, Homo sapiens
DASNLET

SEQ ID NO: 430 - protein, 32 aa, Homo sapiens
GVPSRFSGSGSGTDFTFTISSLQPEDIATYYC

SEQ ID NO: 431 - protein, 7 aa, Homo sapiens
QQYDNLP

SEQ ID NO: 432 - protein, 8 aa, Homo sapiens
RASQGISN

SEQ ID NO: 433 - protein, 7 aa, Homo sapiens
AASTLQS

SEQ ID NO: 434 - protein, 32 aa, Homo sapiens
GVPSRFSGSGSGTDFTLTISSLQPEDVATYYC

SEQ ID NO: 435 - protein, 7 aa, Homo sapiens
QKYNSAP

SEQ ID NO: 436 - protein, 8 aa, Homo sapiens
RASQGIRN

SEQ ID NO: 437 - protein, 32 aa, Homo sapiens
GVPSRFSGSGSGTEFTLTISSLQPEDFATYYC

SEQ ID NO: 438 - protein, 7 aa, Homo sapiens
LQHNSYP

SEQ ID NO: 439 - protein, 23 aa, Homo sapiens
NIQMTQSPSAMSASVGDRVTITC

SEQ ID NO: 440 - protein, 15 aa, Homo sapiens
WFQQKPGKVPKHLIY

SEQ ID NO: 441 - protein, 8 aa, Homo sapiens
RASQGISN

SEQ ID NO: 442 - protein, 15 aa, Homo sapiens
WFQQKPGKAPKSLIY

SEQ ID NO: 443 - protein, 32 aa, Homo sapiens; identical to SEQ ID NO: 426

SEQ ID NO: 444 - protein, 7 aa, Homo sapiens
QQYNSYP

SEQ ID NO: 445 - protein, 8 aa, Homo sapiens
RASQGISS

SEQ ID NO: 446 - protein, 15 aa, Homo sapiens
WYQQKPEKAPKSLIY

SEQ ID NO: 447 - protein, 23 aa, Homo sapiens
AIQLTQSPSSLSASVGDRVTITC

SEQ ID NO: 448 - protein, 15 aa, Homo sapiens
WYQQKPGKAPKLLIY

SEQ ID NO: 449 - protein, 7 aa, Homo sapiens
DASSLES

SEQ ID NO: 450 - protein, 7 aa, Homo sapiens
QQFNSYP

SEQ ID NO: 451 - protein, 23 aa, Homo sapiens
DIQMTQSPSSVSASVGDRVTITC

SEQ ID NO: 452 - protein, 7 aa, Homo sapiens
QQANSFP

SEQ ID NO: 453 - protein, 23 aa, Homo sapiens
DIQLTQSPSFLSASVGDRVTITC

SEQ ID NO: 454 - protein, 7 aa, Homo sapiens
QQLNSYP

SEQ ID NO: 455 - protein, 23 aa, Homo sapiens
AIRMTQSPFSLSASVGDRVTITC

SEQ ID NO: 456 - protein, 8 aa, Homo sapiens
WASQGISS

SEQ ID NO: 457 - protein, 15 aa, Homo sapiens
WYQQKPAKAPKLFIY

SEQ ID NO: 458 - protein, 7 aa, Homo sapiens
YASSLQS

SEQ ID NO: 459 - protein, 32 aa, Homo sapiens
GVPSRFSGSGSGTDYTLTISSLQPEDFATYYC

SEQ ID NO: 460 - protein, 7 aa, Homo sapiens
QQYYSTP

SEQ ID NO: 461 - protein, 23 aa, Homo sapiens
AIRMTQSPSSFSASTGDRVTITC

SEQ ID NO: 462 - protein, 32 aa, Homo sapiens
GVPSRFSGSGSGTDFTLTISCLQSEDFATYYC

SEQ ID NO: 463 - protein, 23 aa, Homo sapiens
VIWMTQSPSLLSASTGDRVTISC

SEQ ID NO: 464 - protein, 8 aa, Homo sapiens
RMSQGISS

SEQ ID NO: 465 - protein, 15 aa, Homo sapiens
WYQQKPGKAPELLIY

SEQ ID NO: 466 - protein, 32 aa, Homo sapiens; identical to SEQ ID NO: 462

SEQ ID NO: 467 - protein, 7 aa, Homo sapiens
QQYYSFP

SEQ ID NO: 468 - protein, 23 aa, Homo sapiens
AIQMTQSPSSLSASVGDRVTITC

SEQ ID NO: 469 - protein, 8 aa, Homo sapiens
RASQGIRN

SEQ ID NO: 470 - protein, 15 aa, Homo sapiens
WYQQKPGKAPKLLIY

SEQ ID NO: 471 - protein, 32 aa, Homo sapiens; identical to SEQ ID NO: 426

SEQ ID NO: 472 - protein, 7 aa, Homo sapiens
LQDYNYP

SEQ ID NO: 473 - protein, 23 aa, Homo sapiens
DIQMTQSPSTLSASVGDRVTITC

SEQ ID NO: 474 - protein, 8 aa, Homo sapiens
RASQSISS

SEQ ID NO: 475 - protein, 32 aa, Homo sapiens
GVPSRFSGSGSGTEFTLTISSLQPDDFATYYC

SEQ ID NO: 476 - protein, 7 aa, Homo sapiens
QQYNSYS

SEQ ID NO: 477 - protein, 23 aa, Homo sapiens
DIVMTQTPLSLPVTPGEPASISC

SEQ ID NO: 478 - protein, 17 aa, Homo sapiens
RSSQSLLDSDDGNTYLD

SEQ ID NO: 479 - protein, 15 aa, Homo sapiens
WYLQKPGQSPQLLIY

SEQ ID NO: 480 - protein, 7 aa, Homo sapiens
TLSYRAS

SEQ ID NO: 481 - protein, 32 aa, Homo sapiens
GVPDRFSGSGSGTDFTLKISRVEAEDVGVYYC

SEQ ID NO: 482 - protein, 7 aa, Homo sapiens
MQRIEFP

SEQ ID NO: 483 - protein, 23 aa, Homo sapiens
DVVMTQSPLSLPVTLGQPASISC

SEQ ID NO: 484 - protein, 9 aa, Homo sapiens
RSSQSLVYS

SEQ ID NO: 485 - protein, 7 aa, Homo sapiens
DGNTYLN

SEQ ID NO: 486 - protein, 15 aa, Homo sapiens
WFQQRPGQSPRRLIY

SEQ ID NO: 487 - protein, 7 aa, Homo sapiens
KVSNRDS

SEQ ID NO: 488 - protein, 7 aa, Homo sapiens
MQGTHWP

SEQ ID NO: 489 - protein, 7 aa, Homo sapiens
KVSNWDS

SEQ ID NO: 490 - protein, 23 aa, Homo sapiens
DIVMTQTPLSLSVTPGQPASISC

SEQ ID NO: 491 - protein, 9 aa, Homo sapiens
KSSQSLLHS

SEQ ID NO: 492 - protein, 7 aa, Homo sapiens
DGKTYLY

SEQ ID NO: 493 - protein, 15 aa, Homo sapiens
WYLQKPGQSPQLLIY

SEQ ID NO: 494 - protein, 7 aa, Homo sapiens
EVSSRFS

SEQ ID NO: 495 - protein, 7 aa, Homo sapiens
MQGIHLP

SEQ ID NO: 496 - protein, 15 aa, Homo sapiens
WYLQKPGQPPQLLIY

SEQ ID NO: 497 - protein, 7 aa, Homo sapiens
EVSNRFS

SEQ ID NO: 498 - protein, 7 aa, Homo sapiens
MQSIQLP

SEQ ID NO: 499 - protein, 23 aa, Homo sapiens
DIVMTQSPLSLPVTPGEPASISC

SEQ ID NO: 500 - protein, 9 aa, Homo sapiens
RSSQSLLHS

SEQ ID NO: 501 - protein, 7 aa, Homo sapiens
NGYNYLD

SEQ ID NO: 502 - protein, 7 aa, Homo sapiens
LGSNRAS

SEQ ID NO: 503 - protein, 7 aa, Homo sapiens
MQALQTP

SEQ ID NO: 504 - protein, 23 aa, Homo sapiens
DIVMTQTPLSSPVTLGQPASISC

SEQ ID NO: 505 - protein, 9 aa, Homo sapiens
RSSQSLVHS

SEQ ID NO: 506 - protein, 7 aa, Homo sapiens
DGNTYLS

SEQ ID NO: 507 - protein, 15 aa, Homo sapiens
WLQQRPGQPPRLLIY

SEQ ID NO: 508 - protein, 7 aa, Homo sapiens
KISNRFS

SEQ ID NO: 509 - protein, 32 aa, Homo sapiens
GVPDRFSGSGAGTDFTLKISRVEAEDVGVYYC

SEQ ID NO: 510 - protein, 7 aa, Homo sapiens
MQATQFP

SEQ ID NO: 511 - protein, 23 aa, Homo sapiens
EIVLTQSPGTLSLSPGERATLSC

SEQ ID NO: 512 - protein, 9 aa, Homo sapiens
RASQSVSSS

SEQ ID NO: 513 - protein, 15 aa, Homo sapiens
WYQQKPGQAPRLLIY

SEQ ID NO: 514 - protein, 7 aa, Homo sapiens
GASSRAT

SEQ ID NO: 515 - protein, 32 aa, Homo sapiens
GIPDRFSGSGSGTDFTLTISRLEPEDFAVYYC

SEQ ID NO: 516 - protein, 7 aa, Homo sapiens
QQYGSSP

SEQ ID NO: 517 - protein, 23 aa, Homo sapiens
EIVLTQSPATLSLSPGERATLSC

SEQ ID NO: 518 - protein, 9 aa, Homo sapiens
GASQSVSSS

SEQ ID NO: 519 - protein, 15 aa, Homo sapiens
WYQQKPGLAPRLLIY

SEQ ID NO: 520 - protein, 7 aa, Homo sapiens
DASSRAT

SEQ ID NO: 521 - protein, 23 aa, Homo sapiens
EIVMTQSPATLSVSPGERATLSC

SEQ ID NO: 522 - protein, 8 aa, Homo sapiens
RASQSVSS

SEQ ID NO: 523 - protein, 7 aa, Homo sapiens
GASTRAT

SEQ ID NO: 524 - protein, 32 aa, Homo sapiens
GIPARFSGSGSGTEFTLTISSLQSEDFAVYYC

SEQ ID NO: 525 - protein, 7 aa, Homo sapiens
QQYNNWP

SEQ ID NO: 526 - protein, 23 aa, Homo sapiens
EIVLTQSPATLSLSPGERATLSC

SEQ ID NO: 527 - protein, 7 aa, Homo sapiens
DASNRAT

SEQ ID NO: 528 - protein, 32 aa, Homo sapiens
GIPARFSGSGSGTDFTLTISSLEPEDFAVYYC

SEQ ID NO: 529 - protein, 7 aa, Homo sapiens
QQRSNWP

SEQ ID NO: 530 - protein, 8 aa, Homo sapiens
RASQGVSS

SEQ ID NO: 531 - protein, 32 aa, Homo sapiens
GIPARFSGSGPGTDFTLTISSLEPEDFAVYYC

SEQ ID NO: 532 - protein, 7 aa, Homo sapiens
QQRSNWH

SEQ ID NO: 533 - protein, 32 aa, Homo sapiens
GIPARFSGSGSGTDFTLTISSLQPEDFAVYYC

SEQ ID NO: 534 - protein, 7 aa, Homo sapiens
QQDYNLP

SEQ ID NO: 535 - protein, 23 aa, Homo sapiens
DIVMTQSPDSLAVSLGERATINC

SEQ ID NO: 536 - protein, 17 aa, Homo sapiens
KSSQSVLYSSNNKNYLA

SEQ ID NO: 537 - protein, 15 aa, Homo sapiens
WYQQKPGQPPKLLIY

SEQ ID NO: 538 - protein, 7 aa, Homo sapiens
WASTRES

SEQ ID NO: 539 - protein, 32 aa, Homo sapiens
GVPDRFSGSGSGTDFTLTISSLQAEDVAVYYC

SEQ ID NO: 540 - protein, 7 aa, Homo sapiens
QQYYSTP

SEQ ID NO: 541 - protein, 23 aa, Homo sapiens
ETTLTQSPAFMSATPGDKVNISC

SEQ ID NO: 542 - protein, 8 aa, Homo sapiens
KASQDIDD

SEQ ID NO: 543 - protein, 15 aa, Homo sapiens
WYQQKPGEAAIFIIQ

SEQ ID NO: 544 - protein, 7 aa, Homo sapiens
EATTLVP

SEQ ID NO: 545 - protein, 32 aa, Homo sapiens
GIPPRFSGSGYGTDFTLTINNIESEDAAYYFC

SEQ ID NO: 546 - protein, 7 aa, Homo sapiens
LQHDNFP

SEQ ID NO: 547 - protein, 23 aa, Homo sapiens
EIVLTQSPDFQSVTPKEKVTITC

SEQ ID NO: 548 - protein, 8 aa, Homo sapiens
RASQSIGS

SEQ ID NO: 549 - protein, 15 aa, Homo sapiens
WYQQKPDQSPKLLIK

SEQ ID NO: 550 - protein, 7 aa, Homo sapiens
YASQSFS

SEQ ID NO: 551 - protein, 32 aa, Homo sapiens
GVPSRFSGSGSGTDFTLTINSLEAEDAATYYC

SEQ ID NO: 552 - protein, 7 aa, Homo sapiens
HQSSSLP

SEQ ID NO: 553 - protein, 23 aa, Homo sapiens
DVVMTQSPAFLSVTPGEKVTITC

SEQ ID NO: 554 - protein, 8 aa, Homo sapiens
QASEGIGN

SEQ ID NO: 555 - protein, 15 aa, Homo sapiens
WYQQKPDQAPKLLIK

SEQ ID NO: 556 - protein, 7 aa, Homo sapiens
YASQSIS

SEQ ID NO: 557 - protein, 32 aa, Homo sapiens
GVPSRFSGSGSGTDFTFTISSLEAEDAATYYC

SEQ ID NO: 558 - protein, 7 aa, Homo sapiens
QQGNKHP

SEQ ID NO: 559 - protein, 22 aa, Homo sapiens
QSVLTQPPSVSEAPRQRVTISC

SEQ ID NO: 560 - protein, 10 aa, Homo sapiens
SGSSSNIGNN

SEQ ID NO: 561 - protein, 15 aa, Homo sapiens
WYQQLPGKAPKLLIY

SEQ ID NO: 562 - protein, 5 aa, Homo sapiens
DLLPS

SEQ ID NO: 563 - protein, 12 aa, Homo sapiens
GVSDRFSGSKSG

SEQ ID NO: 564 - protein, 20 aa, Homo sapiens
TSASLAISGLQSEDEADYYC

SEQ ID NO: 565 - protein, 9 aa, Homo sapiens
AAWDDSLNG

SEQ ID NO: 566 - protein, 22 aa, Homo sapiens
QSVLTQPPSVSGAPGQRVTISC

SEQ ID NO: 567 - protein, 14 aa, Homo sapiens
TGSSSNIGAGYDVH

SEQ ID NO: 568 - protein, 15 aa, Homo sapiens
WYQQLPGTAPKLLIY

SEQ ID NO: 569 - protein, 5 aa, Homo sapiens
SNRPS

SEQ ID NO: 570 - protein, 12 aa, Homo sapiens
GVPDRFSGSKSG

SEQ ID NO: 571 - protein, 20 aa, Homo sapiens
TSASLAITGLQAEDEADYYC

SEQ ID NO: 572 - protein, 9 aa, Homo sapiens
QSYDSSLSG

SEQ ID NO: 573 - protein, 22 aa, Homo sapiens
QSVLTQPPSASGTPGQRVTISC

SEQ ID NO: 574 - protein, 10 aa, Homo sapiens
SGSSSNIGSN

SEQ ID NO: 575 - protein, 5 aa, Homo sapiens
NQRPS

SEQ ID NO: 576 - protein, 20 aa, Homo sapiens
TSASLAISGLRSEDEADYYC

SEQ ID NO: 577 - protein, 9 aa, Homo sapiens
AAWDDSLSG

SEQ ID NO: 578 - protein, 22 aa, Homo sapiens
QSVLTQPPSVSAAPGQKVTISC

SEQ ID NO: 579 - protein, 5 aa, Homo sapiens
NKRPS

SEQ ID NO: 580 - protein, 12 aa, Homo sapiens
GIPDRFSGSKSG

SEQ ID NO: 581 - protein, 20 aa, Homo sapiens
TSATLGITGLQTGDEADYYC

SEQ ID NO: 582 - protein, 9 aa, Homo sapiens
GTWDSSLSA

SEQ ID NO: 583 - protein, 22 aa, Homo sapiens
QSALTQPPSASGSPGQSVTISC

SEQ ID NO: 584 - protein, 14 aa, Homo sapiens
TGTSSDVGGYNYVS

SEQ ID NO: 585 - protein, 15 aa, Homo sapiens
WYQQHPGKAPKLMIY

SEQ ID NO: 586 - protein, 5 aa, Homo sapiens
SKRPS

SEQ ID NO: 587 - protein, 20 aa, Homo sapiens
NTASLTVSGLQAEDEADYYC

SEQ ID NO: 588 - protein, 9 aa, Homo sapiens
SSYAGSNNF

SEQ ID NO: 589 - protein, 22 aa, Homo sapiens
QSALTQPRSVSGSPGQSVTISC

SEQ ID NO: 590 - protein, 20 aa, Homo sapiens
NTASLTISGLQAEDEADYYC

SEQ ID NO: 591 - protein, 9 aa, Homo sapiens
CSYAGSYTF

SEQ ID NO: 592 - protein, 22 aa, Homo sapiens
QSALTQPASVSGSPGQSITISC

SEQ ID NO: 593 - protein, 5 aa, Homo sapiens
SNRPS

SEQ ID NO: 594 - protein, 12 aa, Homo sapiens
GVSNRFSGSKSG

SEQ ID NO: 595 - protein, 9 aa, Homo sapiens
SSYTSSSTL

SEQ ID NO: 596 - protein, 22 aa, Homo sapiens
QSALTQPPSVSGSPGQSVTISC

SEQ ID NO: 597 - protein, 14 aa, Homo sapiens
TGTSSDVGSYNRVS

SEQ ID NO: 598 - protein, 15 aa, Homo sapiens
WYQQPPGTAPKLMIY

SEQ ID NO: 599 - protein, 9 aa, Homo sapiens
SLYTSSSTF

SEQ ID NO: 600 - protein, 14 aa, Homo sapiens
TGTSSDVGSYNLVS

SEQ ID NO: 601 - protein, 12 aa, Homo sapiens
GVSNRFSGSKSG

SEQ ID NO: 602 - protein, 9 aa, Homo sapiens
CSYAGSSTF

SEQ ID NO: 603 - protein, 22 aa, Homo sapiens
SYELTQPPSVSVSPGQTASITC

SEQ ID NO: 604 - protein, 4 aa, Homo sapiens
LGDK

SEQ ID NO: 605 - protein, 15 aa, Homo sapiens
WYQQKPGQSPVLVIY

SEQ ID NO: 606 - protein, 12 aa, Homo sapiens
GIPERFSGSNSG

SEQ ID NO: 607 - protein, 20 aa, Homo sapiens
NTATLTISGTQAMDEADYYC

SEQ ID NO: 608 - protein, 8 aa, Homo sapiens
QAWDSSTA

SEQ ID NO: 609 - protein, 22 aa, Homo sapiens
SYELTQPLSVSVALGQTARITC

SEQ ID NO: 610 - protein, 4 aa, Homo sapiens
IGSK

SEQ ID NO: 611 - protein, 15 aa, Homo sapiens
WYQQKPGQAPVLVIY

SEQ ID NO: 612 - protein, 20 aa, Homo sapiens
NTATLTISRAQAGDEADYYC

SEQ ID NO: 613 - protein, 8 aa, Homo sapiens
QVWDSSTA

SEQ ID NO: 614 - protein, 22 aa, Homo sapiens
SYELTQPPSVSVSPGQTARITC

SEQ ID NO: 615 - protein, 4 aa, Homo sapiens
LPKK

SEQ ID NO: 616 - protein, 15 aa, Homo sapiens
WYQQKSGQAPVLVIY

SEQ ID NO: 617 - protein, 12 aa, Homo sapiens
GIPERFSGSSSG

SEQ ID NO: 618 - protein, 20 aa, Homo sapiens
TMATLTISGAQVEDEADYYC

SEQ ID NO: 619 - protein, 9 aa, Homo sapiens
YSTDSSGNH

SEQ ID NO: 620 - protein, 22 aa, Homo sapiens
SYELTQPPSVSVSLGQMARITC

SEQ ID NO: 621 - protein, 15 aa, Homo sapiens
WYQQKPGQFPVLVIY

SEQ ID NO: 622 - protein, 12 aa, Homo sapiens
GIPERFSGSSSG

SEQ ID NO: 623 - protein, 20 aa, Homo sapiens
TIVTLTISGVQAEDEADYYC

SEQ ID NO: 624 - protein, 9 aa, Homo sapiens
LSADSSGTY

SEQ ID NO: 625 - protein, 22 aa, Homo sapiens
SSELTQDPAVSVALGQTVRITC

SEQ ID NO: 626 - protein, 4 aa, Homo sapiens
LRSY

SEQ ID NO: 627 - protein, 5 aa, Homo sapiens
NNRPS

SEQ ID NO: 628 - protein, 12 aa, Homo sapiens
GIPDRFSGSSSG

SEQ ID NO: 629 - protein, 20 aa, Homo sapiens
NTASLTITGAQAEDEADYYC

SEQ ID NO: 630 - protein, 9 aa, Homo sapiens
NSRDSSGNH

SEQ ID NO: 631 - protein, 22 aa, Homo sapiens
SYVLTQPPSVSVAPGKTARITC

SEQ ID NO: 632 - protein, 5 aa, Homo sapiens
SDRPS

SEQ ID NO: 633 - protein, 20 aa, Homo sapiens
NTATLTISRVEAGDEADYYC

SEQ ID NO: 634 - protein, 9 aa, Homo sapiens
QVWDSSSDH

SEQ ID NO: 635 - protein, 22 aa, Homo sapiens
SYELTQLPSVSVSPGQTARITC

SEQ ID NO: 636 - protein, 4 aa, Homo sapiens
LGEN

SEQ ID NO: 637 - protein, 15 aa, Homo sapiens
WYQQKPGQAPELVIY

SEQ ID NO: 638 - protein, 5 aa, Homo sapiens
SERYP

SEQ ID NO: 639 - protein, 12 aa, Homo sapiens
GIPERFSGSTSG

SEQ ID NO: 640 - protein, 20 aa, Homo sapiens
NTTTLTISRVLTEDEADYYC

SEQ ID NO: 641 - protein, 7 aa, Homo sapiens
LSGDEDN

SEQ ID NO: 642 - protein, 22 aa, Homo sapiens
SYELMQPPSVSVSPGQTARITC

SEQ ID NO: 643 - protein, 4 aa, Homo sapiens
LPKQ

SEQ ID NO: 644 - protein, 20 aa, Homo sapiens
TTVTLTISGVQAEDEADYYC

SEQ ID NO: 645 - protein, 9 aa, Homo sapiens
QSADSSGTY

SEQ ID NO: 646 - protein, 22 aa, Homo sapiens
SYELTQPSSVSVSPGQTARITC

SEQ ID NO: 647 - protein, 4 aa, Homo sapiens
LAKK

SEQ ID NO: 648 - protein, 15 aa, Homo sapiens
WFQQKPGQAPVLVIY

SEQ ID NO: 649 - protein, 20 aa, Homo sapiens
TTVTLTISGAQVEDEADYYC

SEQ ID NO: 650 - protein, 7 aa, Homo sapiens
YSAADNN

SEQ ID NO: 651 - protein, 22 aa, Homo sapiens
LPVLTQPPSASALLGASIKLTC

SEQ ID NO: 652 - protein, 9 aa, Homo sapiens
TLSSEHSTY

SEQ ID NO: 653 - protein, 15 aa, Homo sapiens
WYQQRPGRSPQYIMK

SEQ ID NO: 654 - protein, 8 aa, Homo sapiens
DGSHSKGD

SEQ ID NO: 655 - protein, 12 aa, Homo sapiens
GIPDRFMGSSSG

SEQ ID NO: 656 - protein, 20 aa, Homo sapiens
ADRYLTFSNLQSDDEAEYHC

SEQ ID NO: 657 - protein, 11 aa, Homo sapiens
GESHTIDGQVG

SEQ ID NO: 658 - protein, 22 aa, Homo sapiens
QPVLTQSSSASASLGSSVKLTC

SEQ ID NO: 659 - protein, 9 aa, Homo sapiens
TLSSGHSSY

SEQ ID NO: 660 - protein, 5 aa, Homo sapiens
NQRPS

SEQ ID NO: 661 - protein, 34 aa, Homo sapiens
GVPDRFSGSIDSSSNSASLTISGLKTEDEADYYC

SEQ ID NO: 662 - protein, 7 aa, Homo sapiens
QSYDSSN

SEQ ID NO: 663 - protein, 22 aa, Homo sapiens
QTVVTQEPSLTVSPGGTVTLTC

SEQ ID NO: 664 - protein, 14 aa, Homo sapiens
ASSTGAVTSGYYPN

SEQ ID NO: 665 - protein, 15 aa, Homo sapiens
WFQQKPGQAPRALIY

SEQ ID NO: 666 - protein, 5 aa, Homo sapiens
SNKHS

SEQ ID NO: 667 - protein, 12 aa, Homo sapiens
WTPARFSGSLLG

SEQ ID NO: 668 - protein, 20 aa, Homo sapiens
GKAALTLSGVQPEDEAEYYC

SEQ ID NO: 669 - protein, 8 aa, Homo sapiens
LLYYGGAQ

SEQ ID NO: 670 - protein, 15 aa, Homo sapiens
WHQQQPGKAPRYLMK

SEQ ID NO: 671 - protein, 8 aa, Homo sapiens
SGSYNKGS

SEQ ID NO: 672 - protein, 12 aa, Homo sapiens
GVPDRFSGSSSG

SEQ ID NO: 673 - protein, 20 aa, Homo sapiens
ADRYLTISNLQLEDEADYYC

SEQ ID NO: 674 - protein, 7 aa, Homo sapiens
ETWDSNT

SEQ ID NO: 675 - protein, 22 aa, Homo sapiens
QLVLTQSPSASASLGASVKLTC

SEQ ID NO: 676 - protein, 15 aa, Homo sapiens
WHQQQPEKGPRYLMK

SEQ ID NO: 677 - protein, 12 aa, Homo sapiens
GIPDRFSGSSSG

SEQ ID NO: 678 - protein, 20 aa, Homo sapiens
AERYLTISSLQSEDEADYYC

SEQ ID NO: 679 - protein, 7 aa, Homo sapiens
QTWGTGI

SEQ ID NO: 680 - protein, 22 aa, Homo sapiens
QPVLTQPPSSSASPGESARLTC

SEQ ID NO: 681 - protein, 14 aa, Homo sapiens
TLPSDINVGSYNIY

SEQ ID NO: 682 - protein, 15 aa, Homo sapiens
WYQQKPGSPPRYLLY

SEQ ID NO: 683 - protein, 8 aa, Homo sapiens
DSDKGQGS

SEQ ID NO: 684 - protein, 34 aa, Homo sapiens
GVPSRFSGSKDASANTGILLISGLQSEDEADYYC

SEQ ID NO: 685 - protein, 8 aa, Homo sapiens
MIWPSNAS

SEQ ID NO: 686 - protein, 22 aa, Homo sapiens
QAVLTQPASLSASPGASASLTC

SEQ ID NO: 687 - protein, 14 aa, Homo sapiens
TLRSGINVGTYRIY

SEQ ID NO: 688 - protein, 15 aa, Homo sapiens
WYQQKPGSPPQYLLR

SEQ ID NO: 689 - protein, 8 aa, Homo sapiens
DSDKQQGS

SEQ ID NO: 690 - protein, 34 aa, Homo sapiens
GVPSRFSGSKDASANAGILLISGLQSEDEADYYC

SEQ ID NO: 691 - protein, 8 aa, Homo sapiens
MIWHSSAS

SEQ ID NO: 692 - protein, 22 aa, Homo sapiens
QPVLTQPSSHSASSGASVRLTC

SEQ ID NO: 693 - protein, 14 aa, Homo sapiens
MLSSGFSVGDFWIR

SEQ ID NO: 694 - protein, 15 aa, Homo sapiens
WYQQKPGNPPRYLLY

SEQ ID NO: 695 - protein, 8 aa, Homo sapiens
DSNKGQGS

SEQ ID NO: 696 - protein, 34 aa, Homo sapiens
GVPSRFSGSNDASANAGILRISGLQPEDEADYYC

SEQ ID NO: 697 - protein, 9 aa, Homo sapiens
GTWHSNSKT

SEQ ID NO: 698 - protein, 22 aa, Homo sapiens
NFMLTQPHSVSESPGKTVTISC

SEQ ID NO: 699 - protein, 10 aa, Homo sapiens
TRSSGSIASN

SEQ ID NO: 700 - protein, 15 aa, Homo sapiens
WYQQRPGSSPTTVIY

SEQ ID NO: 701 - protein, 22 aa, Homo sapiens
QAVVTQEPSLTVSPGGTVTLTC

SEQ ID NO: 702 - protein, 14 aa, Homo sapiens
GSSTGAVTSGHYPY

SEQ ID NO: 703 - protein, 15 aa, Homo sapiens
WFQQKPGQAPRTLIY

SEQ ID NO: 704 - protein, 20 aa, Homo sapiens
GKAALTLSGAQPEDEAEYYC

SEQ ID NO: 705 - protein, 8 aa, Homo sapiens
LLSYSGAR

SEQ ID NO: 706 - protein, 22 aa, Homo sapiens
QTVVTQEPSFSVSPGGTVTLTC

SEQ ID NO: 707 - protein, 14 aa, Homo sapiens
GLSSGSVSTSYYPS

SEQ ID NO: 708 - protein, 15 aa, Homo sapiens
WYQQTPGQAPRTLIY

SEQ ID NO: 709 - protein, 5 aa, Homo sapiens
NTRSS

SEQ ID NO: 710 - protein, 12 aa, Homo sapiens
GVPDRFSGSILG

SEQ ID NO: 711 - protein, 20 aa, Homo sapiens
NKAALTITGAQADDESDYYC

SEQ ID NO: 712 - protein, 8 aa, Homo sapiens
VLYMGSGI

SEQ ID NO: 713 - protein, 22 aa, Homo sapiens
QPVLTQPPSASASLGASVTLTC

SEQ ID NO: 714 - protein, 9 aa, Homo sapiens
TLSSGYSNY

SEQ ID NO: 715 - protein, 15 aa, Homo sapiens
WYQQRPGKGPRFVMR

SEQ ID NO: 716 - protein, 12 aa, Homo sapiens
VGTGGIVGSKGD

SEQ ID NO: 717 - protein, 12 aa, Homo sapiens
GIPDRFSVLGSG

SEQ ID NO: 718 - protein, 20 aa, Homo sapiens
LNRYLTIKNIQEEDESDYHC

SEQ ID NO: 719 - protein, 11 aa, Homo sapiens
GADHGSGSNFV

SEQ ID NO: 720 - protein, 22 aa, Homo sapiens
QAGLTQPPSVSKGLRQTATLTC

SEQ ID NO: 721 - protein, 10 aa, Homo sapiens
TGNSNNVGNQ

SEQ ID NO: 722 - protein, 15 aa, Homo sapiens
WLQQHQGHPPKLLSY

SEQ ID NO: 723 - protein, 12 aa, Homo sapiens
GISERLSASRSG

SEQ ID NO: 724 - protein, 20 aa, Homo sapiens
NTASLTITGLQPEDEADYYC

SEQ ID NO: 725 - protein, 9 aa, Homo sapiens
SAWDSSLSA

SEQ ID NO: 726 - protein, 15 aa, Artificial
  Linker peptide sequence
GGGGSGGGGSGGGGS

(19) United States
(12) Patent Application Publication
(10) Pub. No.: US 2010/0168393 A1
(43) Pub. Date: Jul. 1, 2010

(54) Antibody Polypeptide Libray Screening and Selected Antibody Polypeptides

(75) Inventors: Jasper Clube, Cambridge (GB); Laurent Jespers, Cambridge (GB)

Correspondence Address: EDWARDS ANGELL PALMER & DODGE LLP, P.O. BOX 55874, BOSTON, MA, 02205, US

(73) Assignee: Big Glucose Ltd., Ramat-Gan (IL)

(21) Appl. No.: 12/083,368

(22) PCT Filed: Oct. 11, 2006
(86) PCT No.: PCT/GB2006/003781

§ 371 Date: Dec. 22, 2009

(30) Foreign Application Priority Data
Oct. 11, 2005 (GB) 0520644.6
Oct. 18, 2005 (GB) 0521140.4

(51) Int. Cl. C07K 16/00; C40B 30/04

(52) U.S. Cl. 530/387.3; 506/9; 530/387.1

(57) ABSTRACT

The present invention provides further developments in the screening of antibody polypeptide libraries. The invention also provides novel isolated antibody polypeptides obtainable by the methods of the invention.

The present invention provides further developments in the screening of antibody polypeptide libraries. The invention also provides novel isolated antibody polypeptides obtainable by the methods of the invention.

WO99/20749, which is assigned to the present applicants, describes methods of screening an antibody library (e.g. a library of antibody heavy chain variable domains) using a generic ligand, e.g. an antibody or an antibody fragment, or any substance comprising one or more specific binding sites from an antibody. “Antibodies” are defined as constructions using the binding (variable) region of such antibodies. The disclosure of WO99/20749 is incorporated herein by reference, particularly the disclosure of library generation (“Construction of libraries of the invention”), selection of polypeptides from libraries (“Selection of polypeptides according to the invention”), ligand choice (“Antibodies for use as ligands in polypeptide selection”) and industrial application (“Use of polypeptides selected according to the invention”). All these sections are explicitly incorporated into the present application to provide disclosure of features that may be used in the present invention, and the skilled person will readily recognise—in the context of the claims of the present application—those features that may be used in the present invention.

A class of non-conventional antibodies—heavy chain antibodies—has been described in the literature. These antibodies have been found in high titers in the serum of patients with heavy-chain disease, in EBV transformed B-cells, and more importantly in the serum of Camelidae (camels, llamas). These heavy-chain antibodies comprise no light chains but only a single pair of identical heavy chains. These heavy-chains differ to those of conventional IgGs in that they lack the constant domain 1 (CH1) which plays a role in mediating light chain pairing on heavy chain. Consequently, the resulting heavy chain antibodies comprise no light chain variable domain but only two unpaired heavy-chain variable domains. Studies on the sera of immunized Camelidae have shown that despite the absence of a light chain, these heavy chain antibodies do specifically bind antigens with moderate to high affinity. The unpaired heavy-chain variable domain (named VHH) has undergone genetic adaptation throughout evolution: a number of amino acid substitutions have taken place (even at the germline level) in the part that normally interacts with the VL domain: L45 conserved in VHs is substituted by Arg. Other positions are frequently mutated: V37 into Phe, G44 into Glu and W47 into Gly. Finally, the CDR3 of VHHs from camels (but not llamas) are on average longer (16-17 amino acids) than those of murine and human VHS (9 and 12 amino acids, respectively) thereby compensating for the absence of a VL domain for binding to an antigen. Reference is made to WO05044858A1, WO04062551A2, WO04041867A2, WO04041865A2, WO04041863A2, WO04041862A2, WO03050531A2 and EP0656946 for a description of Camelid VHH domains.

Since 1989, it has been recognized single variable domains of antibodies have therapeutic potential. Due to their small size they can dock onto poorly accessible antigenic sites (clefts, canyons, active sites) for conventional antibodies. These domains can also be formatted into a range of products tailored to the needs: e.g. they can be multimerized (either chemically or genetically) to increase avidity whilst keeping a relatively small overall size. The persistence in serum can be adjusted through PEGylation (to increase the hydrodynamic size) or through covalent or non-covalent binding to serum protein such as serum albumin which exhibits prolonged half-lifes (up to 19 days in man). Finally single variable domains can be re-implemented into IgG in order to benefit from the Fc-effector functions. In all cases, access to the genetic information of the antigen-specific antibody variable domains is an absolute prerequisite to all above mentioned strategies.

Several methods have been developed to isolate the genes encoding the antibody variable domains of antigen-specific antibodies. One of these methods is based on the early observation that during its development each B-lymphocyte expresses a single type of antibody on its cell surface (attachment is mediated by a genetic fusion to a membrane-anchoring peptide). Binding to antigen (and participation of T helper cells) mediates proliferation of antigen-specific of B-lymphocytes which in a later stage mature into plasma cells. These cells do not express surface-bound antibodies but rather secrete these in high quantities. Thus, in the course of their development, B-lymphocytes can be viewed as genetic display packages where the phenotype (the antibody) is linked to the genotype (the antibody genes). Therefore in order to isolate the genes encoding antigen-specific antibodies, methods have been developed whereby (i) a collection of B-lymphocytes (isolated from an immunized animal) is contacted with the antigen (which can be either immobilized on cells or on a solid phase, or which can be dye-labelled), (ii) antigen-specific B-lymphocytes bind to the antigen, (iii) bound antigen-specific B-lymphocytes are separated from unbound B-lymphocytes, (iv) bound antigen-specific B-lymphocytes are recovered into receptacles, tubes, wells or dishes and (v) the genes encoding the variable domains of antigen-specific antibodies are recovered from the isolated B-lymphocytes (which can be stored as monoclonal or polyclonal populations). Examples of methods using this scheme are those described by
(i) Babcook et al. (1996) Proc. Natl. Acad. Sci. USA 93, 7843-7848.
   SLAM overcomes the limitations of both hybridoma technology and bacterially expressed antibody libraries by enabling high affinity antibodies generated during in vivo immune responses to be isolated from any species. SLAM enables a single lymphocyte that is producing an antibody with a desired specificity or function to be identified within a large population of lymphoid cells and the genetic information that encodes the specificity of the antibody to be rescued from that lymphocyte, e.g. to enable for cloning of the genetic information into an expression vector to enable expression of large quantities of the antibody. In one embodiment, antibody producing cells which produce antibodies which bind to selected antigens and detected using an adapted haemolytic plaque assay method (Jerre and Nordin (1963) Science 140, 405). In this assay erythrocytes are coated with the selected antigen and incubated with the population of antibody producing cells and a source of complement. Single antibody producing cells are identified by the formation of haemolytic plaques. Plaques of lysed erythrocytes are identified using an inverted microscope and the single antibody producing cell of interest at the centre of the plaque is removed using micromanipulation techniques, then used to seed a single well coated with EL4-B5 T cells (which provide vital cell-cell interactions and soluble factors for B-cell expansion). On average 0.3 B-cell were seeded per well to ensure clonal distribution. The antibody genes from these clonally expanded B cells are then cloned by reverse transcription PCR. Other methods for detecting single antibody-producing cells of a desired function have already been described in International Patent Specification, WO 92/02551.
(ii) de Wildt et al. (1997) J. Immunol. Methods 2073, 61-67.
   The described method comprises the following steps: (i) collection of B-lymphocytes from human donors, (ii) detection of CD19+/CD20+ cells in a Fluorescence Activated Flow Cytometer (FACS instrument) equipped with an automatic cell dispensing unit, (iii) dispensing each CD19+/CD20+ B cells in a single well coated with EL4-B5 T cells which provide cell-cell interactions (CD40-CD40L) as well as soluble growth factors (iv) expansion of each single B-cell to increase the amount of mRNA, and (v) recovery of the antibody genes by RT-PCR. It should be noted that step 4 (B-cell expansion) is optional provided that PCR methodologies can be designed to efficiently PCR amplify antibody genes from single cells.
(iii) Lawson et al. WO 2004106377.
   In this patent application, the authors have isolated antigen-specific B-cells by flow cytometry (de Wildt et al. (1997) J. Immunol. Methods, 2073, 61-67) or biopanning (Lagerkvist et al. (1995) Biotechniques 18, 862-869). In contrast to these two approaches, the method by Lawson et al. does not require the isolation of individual B cells. The number of B cells per well ranges from 100 to 20,000—yet following B-cell expansion, RT-PCR of the antibody variable genes reveals a monoclonal antibody sequence in most wells.

All these methods use the antigen to isolate a sub-population of antibody-presenting B-cells. This approach does not take into account that within an antigen-specific B-cell population, the displayed antibodies may differ in regions that are not directly involved in antigen-binding. For example, conventional antibodies are made either from the kappa or the lambda light chain. Selection based on the antigen only will therefore not allow sorting of the different B-cell subpopulations. Each antigen-selected B-lymphocyte will then have to be tested thereafter for the light chain isotype. A more important example is given by observations on immunized Camelidae: in Camels, ˜20% of the B-lymphocytes are thought to express on their surface and/or secrete heavy-chain antibodies. The remainder of B-lymphocytes display/express conventional four-chain antibodies. Thus antigen-based selection does not allow to discriminate between those B-cells expressing heavy-chain antibodies from those B-cells expressing conventional antibodies. This problem can be solved by the present invention.

Reference is also made to the following publications in the name of Celltech:
WO04051268A1: ASSAY FOR IDENTIFYING ANTIBODY PRODUCING CELLS, which discloses a homogeneous assay for identifying an antibody-producing cell producing an antibody which binds to a selected antigen, the method comprises incubating antibody producing cells with an antigen and a labelled anti-antibody antibody.
WO04106377A1: METHODS FOR PRODUCING ANTIBODIES, which discloses obtaining an antibody with a desired function, by contacting B cells with a capturing agent, separating the captured B cells and culturing and screening the captured B cells to identify cells that the produce antibody to obtain the desired antibody.
WO05019823A1: METHODS FOR OBTAINING ANTIBODIES, which discloses enriching a B cell population for cells producing an antibody that recognises an antigen of interest, by contacting the cells, an antigen of interest and an antibody-particle complex, where the antibody recognizes the antigen of interest.
WO05019824A1: METHODS FOR OBTAINING ANTIBODIES, which discloses enriching a B cell population in cells producing an antibody recognizing an antigen of interest comprises labelling the cells with antibodies specific for a B cell marker and for the antigen, and isolating the doubly labelled cells.

The antigen binding domain of an antibody comprises two separate regions: a heavy chain variable domain (VH) and a light chain variable domain (VL) which can be either Vκ or V_{λ}). The antigen binding site itself is formed by six polypeptide loops: three from VHdomain (l rearrangement of gene segments. The VH gene is produced by the recombinatioH1, H2 and H3) and three from VL domain (L1, L2 and L3). A diverse primary repertoire of V genes that encode the VH and VL domains is produced by the combinatorian of three gene segments, VH, D and JH. In humans, there are approximately 51 functional VH segments (Cook and Tomlinson (1995) Immunol Today, 16: 237), 25 functional D segments (Corbett et al. (1997) J. Mol. Biol., 268: 69) and 6 functional J_{H} segments (Ravetch et al. (1981) Cell, 27: 583), depending on the haplotype. The VH segment encodes the region of the polypeptide chain which forms the first and second antigen binding loops of the VH domain (H1 and H2), whilst the VH, D and J_{H} segments combine to form the third antigen binding loop of the VH domain (H3). The VL gene is produced by the recombination of only two gene segments, VL and JL. In humans, there are approximately 40 functional Vκ segments (Schäble and Zachau (1993) Biol. Chem. Hoppe-Seyler, 374: 1001), 31 functional V_{λ} segments (Williams et al. (1996) J. Mol. Biol., 264: 220; Kawasaki et al. (1997) Genome Res., 7: 250), 5 functional Jκ segments (Hieter et al. (1982) J. Biol. Chem., 257: 1516) and 4 functional J_{λ} segments (Vasicek and Leder (1990) J. Exp. Med., 172: 609), depending on the haplotype. The VL segment encodes the region of the polypeptide chain which forms the first and second antigen binding loops of the VL domain (L1 and L2), whilst the VL and JL segments combine to form the third antigen binding loop of the VL domain (L3). Antibodies selected from this primary repertoire are believed to be sufficiently diverse to bind almost all antigens with at least moderate affinity. High affinity antibodies are produced by “affinity maturation” of the rearranged genes, in which point mutations are generated and selected by the immune system on the basis of improved binding.

Analysis of the structures and sequences of antibodies has shown that five of the six antigen binding loops (H1, H2, L1, L2, and L3) possess a limited number of main-chain conformations or canonical structures (Chothia and Lesk (1987) J. Mol. Biol., 196: 901; Chothia et al. (1989) Nature, 342: 877). The main-chain conformations are determined by (i) the length of the antigen binding loop, and (ii) particular residues, or types of residue, at certain key position in the antigen binding loop and the antibody framework. Analysis of the loop lengths and key residues has enabled us to the predict the main-chain conformations of H1, H2, L1, L2 and L3 encoded by the majority of human antibody sequences (Chothia et al. (1992) J. Mol. Biol., 227: 799; Tomlinson et al. (1995) EMBO J., 14: 4628; Williams et al. (1996) J. Mol. Biol., 264: 220). Although the H3 region is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol., 263: 800; Shirai et al. (1996) FEBS Letters, 399: 1).

A similar analysis of side-chain diversity in human antibody sequences has enabled the separation of the pattern of sequence diversity in the primary repertoire from that created by somatic hypermutation. It was found that the two patterns are complementary: diversity in the primary repertoire is focused at the centre of the antigen binding whereas somatic hypermutation spreads diversity to regions at the periphery that are highly conserved in the primary repertoire (Tomlinson et al. (1996) J. Mol. Biol., 256: 813; Ignatovich et al. (1997) J. Mol. Biol, 268: 69). This complementarity seems to have evolved as an efficient strategy for searching sequence space, given the limited number B cells available for selection at any given time. Thus, antibodies are first selected from the primary repertoire based on diversity at the centre of the binding site. Somatic hypermutation is then left to optimise residues at the periphery without disrupting favourable interactions established during the primary response.

The advent of phage-display technology (Smith (1985) Science, 228: 1315; Scott and Smith (1990) Science, 249: 386; McCafferty et al. (1990) Nature, 348: 552) has enabled the in vitro selection of human antibodies against a wide range of target antigens from “single pot” libraries. These phage-antibody libraries can be grouped into two categories: natural libraries which use rearranged V genes harvested from human B cells (Marks et al. (1991) J. Mol. Biol., 222: 581; Vaughan et al. (1996) Nature Biotech., 14: 309) or synthetic libraries whereby germline V gene segments are ‘rearranged’ in vitro (Hoogenboom & Winter (1992) J. Mol. Biol., 227: 381; Nissim et al. (1994) EMBO J., 13: 692; Griffiths et al. (1994) EMBO J., 13: 3245; De Kruif et al. (1995) J. Mol. Biol., 248: 97) or where synthetic CDRs are incorporated into a single rearranged V gene (Barbas et al. (1992) Proc. Natl. Acad. Sci. USA, 89: 4457). Although synthetic libraries help to overcome the inherent biases of the natural repertoire which can limit the effective size of phage libraries constructed from rearranged V genes, they require the use of long degenerate PCR primers which frequently introduce base-pair deletions into the assembled V genes. This high degree of randomisation may also lead to the creation of antibodies which are unable to fold correctly and are also therefore non-functional. Furthermore, antibodies selected from these libraries may be poorly expressed and, in many cases, will contain framework mutations that may effect the antibodies immunogenicity when used in human therapy.

In an extension of the synthetic library approach it has been suggested (WO97/08320, Morphosys) that human antibody frameworks can be pre-optimised by synthesising a set of ‘master genes’ that have consensus framework sequences and incorporate amino acid substitutions shown to improve folding and expression. Diversity in the CDRs is then incorporated using oligonucleotides. Since it is desirable to produce artificial human antibodies which will not be recognised as foreign by the human immune system, the use of consensus frameworks which, in most cases, do not correspond to any natural framework is a disadvantage of this approach. Furthermore, since it is likely that the CDR diversity will also have an effect on folding and/or expression, it is preferable to optimise the folding and/or expression (and remove any frame-shifts or stop codons) after the V gene has been fully assembled. To this end, it would be desirable to have a selection system which could eliminate non-functional or poorly folded/expressed members of the library before selection with the target antigen is carried out.

A further problem with the libraries of the prior art is that, because the main-chain conformation is heterogeneous, three-dimensional structural modelling is difficult because suitable high resolution crystallographic data may not be available. This is a particular problem for the H3 region, where the vast majority of antibodies derived from natural or synthetic antibody libraries have medium length or long loops and therefore cannot be modelled.

### SUMMARY OF THE INVENTION

According to the first aspect of the present invention, there is provided a method for selecting, from a repertoire of polypeptides, a population of functional polypeptides which bind a target ligand in a first binding site and a generic ligand in a second binding site, which generic ligand is capable of binding functional members of the repertoire regardless of target ligand specificity, comprising the steps of:
a) contacting the repertoire with the generic ligand and selecting functional polypeptides bound thereto; and
b) contacting the selected functional polypeptides with the target ligand and selecting a population of polypeptides which bind to the target ligand.

The invention accordingly provides a method by which a repertoire of polypeptides is preselected, according to functionality as determined by the ability to bind the generic ligand, and the subset of polypeptides obtained as a result of preselection is then employed for further rounds of selection according to the ability to bind the target ligand. Although, in a preferred embodiment, the repertoire is first selected with the generic ligand, it will be apparent to one skilled in the art that the repertoire may be contacted with the ligands in the opposite order, i.e. with the target ligand before the generic ligand.

The invention permits the person skilled in the art to remove, from a chosen repertoire of polypeptides, those polypeptides which are non-functional, for example as a result of the introduction of frame-shift mutations, stop codons, folding mutants or expression mutants which would be or are incapable of binding to substantially any target ligand. Such non-functional mutants are generated by the normal randomisation and variation procedures employed in the construction of polypeptide repertoires. At the same time the invention permits the person skilled in the art to enrich a chosen repertoire of polypeptides for those polypeptides which are functional, well folded and highly expressed.

Preferably, two or more subsets of polypeptides are obtained from a repertoire by the method of the invention, for example, by prescreening the repertoire with two or more generic ligands, or by contacting the repertoire with the generic ligand(s) under different conditions. Advantageously, the subsets of polypeptides thus obtained are combined to form a further repertoire of polypeptides, which may be further screened by contacting with target and/or generic ligands.

Preferably, the library according to the invention comprises polypeptides of the immunoglobulin superfamily, such as antibody polypeptides or T-cell receptor polypeptides. Advantageously, the library may comprise individual immunoglobulin domains, such as the VH or VL domains of antibodies, or the V_{β} or V_{α} domains of T-cell receptors. In a preferred embodiment, therefore, repertoires of, for example, VH and VL polypeptides may be individually prescreened using a generic ligand and then combined to produce a functional repertoire comprising both VH and VL polypeptides. Such a repertoire can then be screened with a target ligand in order to isolate polypeptides comprising both VH and VL domains and having the desired binding specificity.

In an advantageous embodiment, the generic ligand selected for use with immunoglobulin repertoires is a superantigen. Superantigens are able to bind to functional immunoglobulin molecules, or subsets thereof comprising particular main-chain conformations, irrespective of target ligand specificity. Alternatively, generic ligands may be selected from any ligand capable of binding to the general structure of the polypeptides which make up any given repertoire, such as antibodies themselves, metal ion matrices, organic compounds including proteins or peptides, and the like.

In a second aspect, the invention provides a library wherein the functional members have binding sites for both generic and target ligands. Libraries may be specifically designed for this purpose, for example by constructing antibody libraries having a main-chain conformation which is recognised by a given superantigen, or by constructing a library in which substantially all potentially functional members possess a structure recognisable by a antibody ligand.

In a third aspect, the invention provides a method for detecting, immobilising, purifying or immunoprecipitating one or more members of a repertoire of polypeptides previously selected according to the invention, comprising binding the members to the generic ligand.

In a fourth aspect, the invention provides a library comprising a repertoire of polypeptides of the immunoglobulin superfamily, wherein the members of the repertoire have a known main-chain conformation.

In a fifth aspect, the invention provides a method for selecting a polypeptide having a desired generic and/or target ligand binding site from a repertoire of polypeptides, comprising the steps of:
a) expressing a library according to the preceding aspects of the invention;
b) contacting the polypeptides with generic and/or target ligands and selecting those which bind the generic and/or target ligand; and
c) optionally amplifying the selected polypeptide(s) which bind the generic and/or target ligand.
d) optionally repeating steps a)-c).

Repertoires of polypeptides are advantageously both generated and maintained in the form of a nucleic acid library. Therefore, in a sixth aspect, the invention provides a nucleic acid library encoding a repertoire of such polypeptides.

In a seventh aspect, the present invention provides a method for selecting, from a repertoire of antibody polypeptides, a population of functional variable domains which bind a target ligand and a generic ligand, which generic ligand is capable of binding functional members of the repertoire regardless of target ligand specificity, comprising the steps of:
a) contacting the repertoire with said generic ligand and selecting functional variable domains bound thereto; and
b) contacting the selected functional variable domains with the target ligand and selecting a population of variable domains which bind to the target ligand,
wherein either (i) the variable domains are heavy chain variable domains and the generic ligand is an antibody light chain variable domain; or (ii) the variable domains are light chain variable domains and the generic ligand is an antibody heavy chain variable domain; and
wherein optionally in (i) the heavy chain variable domains are Camelid variable domains (VHH) or derived from a Camelid heavy chain antibody (H2 antibody); or optionally in (i) and (ii) each variable domain is a human variable domain or derived from a human.

Preferably, the repertoire of antibody polypeptides is first contacted with the target ligand and then with the generic ligand.

Preferably, the generic ligand binds a subset of the repertoire of variable domains

Preferably, two or more subsets are selected from the repertoire of polypeptides.

Preferably, the selection is performed with two or more generic ligands, optionally two or more light chain variable domains (for option (i)) or two or more heavy chain variable domains (for option (ii)).

Preferably, the two or more subsets are combined after selection to produce a further repertoire of polypeptides.

Preferably, two or more repertoires of polypeptides are contacted with generic ligands and the subsets of polypeptides thereby obtained are then combined.

In one embodiment of the seventh aspect, a population of antibody heavy chain variable domains is selected and a population of antibody light chain variable domains is selected and the populations thereby obtained are then combined.

In an eighth aspect, the present invention also provides a method for selecting, from a repertoire of polypeptides, a population of functional T-cell receptor domains which bind a target ligand and a generic ligand, which generic ligand is capable of binding functional members of the repertoire regardless of target ligand specificity, comprising the steps of:
a) contacting the repertoire with said generic ligand and selecting functional T-cell receptor domains bound thereto; and
b) contacting the selected functional T-cell receptor domains with the target ligand and selecting a population of T-cell receptor domains which bind to the target ligand,
wherein either (i) the T-cell receptor domains are V_{α} domains and the generic ligand is a T-cell receptor V_{β} domain; or (ii) the T-cell domains are T-cell receptor V_{β} domains and the generic ligand is a T-cell receptor V_{α} domain; and
wherein optionally in (i) the T-cell receptor V_{α} domains are Camelid domains derived from a Camelid; or optionally in (i) and (ii) each T-cell receptor domain is a human domain or derived from a human.

In one embodiment of the eighth aspect, a population of T-cell receptor V_{α} domains is selected and a population of T-cell receptor V_{β} domains is selected and the populations thereby obtained are then combined.

In a ninth aspect, the present invention also provides a method for selecting at least one antibody heavy chain variable domain from a population of antibody polypeptides, the method comprising:
a) contacting the population with an antibody light chain variable domain and
b) selecting at least one antibody heavy chain variable domain that binds to the light chain variable domain.

In one embodiment, a target antigen is used to perform SLAM (selected lymphocyte antibody method) on a starting population of antibody polypeptides to select a population of antibody polypeptides that bind the target antigen; and the selected population is used as the population of antibody polypeptides in step a) that is contacted with the light chain variable domain.

Preferably, prior to step a), there is a step of contacting antibody polypeptides with a target ligand and selecting antibody polypeptides that bind the target ligand, thereby providing said population of antibody polypeptides used in step a).

Preferably, after to step b), there is a step of contacting antibody heavy chain variable domains selected in step b) with a target ligand and selecting heavy chain variable domains that bind the target ligand.

Preferably, each heavy chain domain selected in step b) is from the group consisting of heavy chain variable domains derived from a Camelid; a VHH domain; a Nanobody™; a VHH having a glycine at position 44; a VHH having a leucine at position 45; a VHH having a tryptophan at position 47; a VHH having a glycine at position 44 and a leucine at position 45; a VHH having a glycine at position 44 and a tryptophan at position 47; a VHH having a leucine at position 45 and a tryptophan at position 47; a VHH having a glycine at position 44, a leucine at position 45 and a tryptophan at position 47; a VHH having a tryptophan or arginine at position 103.

Preferably, each heavy chain domain selected in step b) is a humanised Camelid or murine heavy chain variable domain or a humanised Nanobody™.

Preferably, each heavy chain domain selected in step b) is a human heavy chain variable domain.

Preferably, the light chain variable domain is a human light chain variable domain or derived from a human or a light chain variable domain having a FW2 sequence that is identical to FW2 encoded by germline gene sequence DPK9.

Preferably, the light chain variable domain is a Camelid light chain variable domain or derived from a Camelid.

Preferably, the population in step a) is provided by a population of B-cells.

Preferably, the B-cells are peripheral blood lymphocytes.

Preferably, the B-cells are isolated from an animal that has been immunised with a target antigen.

Preferably, the B-cells are isolated from an animal that has not been immunised with a target antigen.

Preferably, the population used in step a) is provided by a repertoire of antibody polypeptides encoded by synthetically rearranged antibody genes.

Preferably, the population used in step a) is provided by a phage display library comprising bacteriophage displaying said antibody polypeptides.

Preferably, the population used in step a) comprises (i) antibody polypeptides each comprising at least one heavy chain variable domain that is not paired with a light chain variable domain; and (ii) antibody polypeptides each comprising a heavy chain variable domain that is paired with a light chain variable domain.

Preferably, the population used in step a) comprises Camelid heavy chain single variable domains (VHH) or Nanobodies™.

Preferably, the population used in step a) comprises human heavy chain single variable domains (VH).

Preferably, in step b) at least one of the selected antibody heavy chain variable domains is fused or conjugated to a protein moiety. Preferably, the protein moiety is selected from a bacteriophage coat protein, one or more antibody domains, an antibody Fc domain, an enzyme, a toxin, a label and an effector group.

Preferably, in step b) at least one of the selected antibody heavy chain variable domains is part of an antibody or an antibody fragment selected from an IgG, Fab, Fab′, F(ab)₂, F(ab′)₂, scFv, Fv and a disulphide bonded Fv.

### A Particularly Preferred Embodiment of the Ninth Aspect of the Invention:

In this embodiment, the population in step a) is provided by a population of B-cells, preferably peripheral blood lymphocytes that have been isolated from a Camelid (e.g. llama or camel) that has been immunised with a target antigen. In this embodiment, preferably the target antigen is selected from the group consisting of TNF alpha, serum albumin, von Willebrand's factor (vWF), IgE, interferon gamma, EGFR, IgE, MMP12, PDK1 and Amyloid beta (A-beta), or any one of the targets listed in Annex 1. The light chain variable domain used in step b) is a human light chain variable domain; derived from a human; a light chain variable domain having a FW2 sequence that is identical to FW2 encoded by germline gene sequence DPK9; or a Camelid, rabbit or mouse light chain variable domain. Thus, this embodiment contemplates the use of a peripheral blood lymphocyte population in step a) from an immunised Camelid that is selected using a human light chain variable domain (or a light chain domain at least having a human interface region (this region including amino acids 44-47 according to Kabat), i.e. the region usually interfacing with VH domains in human VH/VL pairings).

An aspect of the invention provides a method for selecting at least one Camelid antibody VHH domain from a population of Camelid antibody polypeptides provided by B-cells isolated from a Camelid that has been immunised with a target antigen, the method comprising:
a) contacting the population with an antibody light chain variable domain and
b) selecting at least one VHH domain that binds to the light chain variable domain.

Preferably, the light chain variable domain is a human light chain variable domain.

Preferably, the B-cells are provided in a plurality of wells or receptacles, wherein each well or receptacle contains on average one B-cell type.

Preferably, in step b) at least one of the selected antibody heavy chain variable domains is fused or conjugated to a protein moiety. Preferably, the protein moiety is selected from a bacteriophage coat protein, one or more antibody domains, an antibody Fc domain, an enzyme, a toxin, a label and an effector group.

Preferably, in step b) at least one of the selected antibody heavy chain variable domains is part of an antibody or an antibody fragment selected from an IgG, Fab, Fab′, F(ab)₂, F(ab′)₂, scFv, Fv and a disulphide bonded Fv.

In a tenth aspect the invention provides an isolated antibody polypeptide comprising or consisting of an antibody heavy chain variable domain, wherein the polypeptide is obtainable by the method of the ninth aspect of the invention, wherein the light chain variable domain in the method is a human light chain variable domain and the heavy chain variable domain is from a non-human mammal. Preferably, the heavy chain variable domain of the antibody polypeptide is from the group consisting of a heavy chain variable domain derived from a Camelid; a VHH domain; a Nanobody™; a VHH having a glycine at position 44; a VHH having a leucine at position 45; a VHH having a tryptophan at position 47; a VHH having a glycine at position 44 and a leucine at position 45; a VHH having a glycine at position 44 and a tryptophan at position 47; a VHH having a leucine at position 45 and a tryptophan at position 47; a VHH having a glycine at position 44, a leucine at position 45 and a tryptophan at position 47; a VHH having a tryptophan or arginine at position 103. Preferably, the heavy chain variable domain of the antibody polypeptide is provided as part of a Camelid IgG or an IgG derived from a Camelid. Preferably, the heavy chain variable domain of the antibody polypeptide is provided as part of a human IgG or an IgG derived from a human, and wherein the heavy chain variable domain is paired in the IgG with a light chain variable domain that is different from the light chain variable domain used in the method of the ninth aspect of the invention. The invention also provides a derivative of the antibody polypeptide, wherein the derivative has a CDR3 mutation as compared to the CDR3 of the variable domain of the antibody polypeptide of the tenth aspect. The invention also provides a derivative that is produced by affinity maturation of an antibody polypeptide of the tenth aspect.

An antibody polypeptide according to the invention (eg, the 10^{th} or 12^{th} aspect) in one embodiment is used as a medicament, or for therapy and/or prevention of a disease or condition in a human.

In one embodiment of a method, use or antibody polypeptide of the invention, the heavy chain variable domain binds a target ligand selected from the group consisting of TNF alpha, serum albumin, von Willebrand's factor (vWF), IgE, interferon gamma, EGFR, IgE, MMP12, PDK1 and Amyloid beta (A-beta), or any one of the targets listed in Annex 1.

In eleventh aspect, the present invention also provides a method for selecting at least one antibody light chain variable domain from a population of antibody polypeptides, the method comprising:
a) contacting the population with an antibody heavy chain variable domain and
b) selecting at least one antibody light chain variable domain that binds to the heavy chain variable domain.

Preferably, prior to step a), there is a step of contacting antibody polypeptides with a target ligand and selecting antibody polypeptides that bind the target ligand, thereby providing said population of antibody polypeptides used in step a).

Preferably, after to step b), there is a step of contacting antibody light chain variable domains selected in step b) with a target ligand and selecting light chain variable domains that bind the target ligand.

Preferably, each light chain domain selected in step b) is derived from a Camelid.

Preferably, each light chain domain selected in step b) is a human light chain variable domain.

Preferably, the heavy chain variable domain is a human heavy chain variable domain; derived from a human; a heavy chain variable domain having a FW2 sequence that is identical to FW2 encoded by germline gene sequence DP47; or a heavy chain variable domain having positions 44, 45 and 47 that are identical to positions 44, 45 and 47 encoded by germline gene sequence DP47.

Preferably, the heavy chain variable domain is a Camelid heavy chain variable domain (VHH or VH) or derived from a Camelid.

Preferably, the population in step a) is provided by a population of B-cells.

Preferably, the B-cells are peripheral blood lymphocytes.

Preferably, the B-cells are isolated from an animal that has been immunised with a target antigen.

Preferably, the B-cells are isolated from an animal that has not been immunised with a target antigen.

Preferably, the population used in step a) is provided by a repertoire of antibody polypeptides encoded by synthetically rearranged antibody genes.

Preferably, the population used in step a) is provided by a phage display library comprising bacteriophage displaying said antibody polypeptides:

Preferably, the population used in step a) comprises (i) antibody polypeptides each comprising at least one light chain variable domain that is not paired with a heavy chain variable domain; and (ii) antibody polypeptides each comprising a light chain variable domain that is paired with a heavy chain variable domain.

Preferably, the population used in step a) comprises human light chain single variable domains (VL).

Preferably, in step b) at least one of the selected antibody light chain variable domains is fused or conjugated to a protein moiety. Preferably, the protein moiety is selected from a bacteriophage coat protein, one or more antibody domains, an antibody Fc domain, an enzyme, a toxin, a label and an effector group.

Preferably, in step b) at least one of the selected antibody heavy chain variable domains is part of an antibody or an antibody fragment selected from an IgG, Fab, Fab′, F(ab)₂, F(ab′)₂, scFv, Fv and a disulphide bonded Fv.

In one embodiment of the eleventh aspect, a target antigen is used to performing SLAM (selected lymphocyte antibody method) on a starting population of antibody polypeptides to select a population of antibody polypeptides that bind the target antigen; and the selected population is used as the population of antibody polypeptides in step a) that is contacted with the heavy chain variable domain.

In a twelfth aspect, the invention provides an isolated antibody polypeptide comprising or consisting of an antibody light chain variable domain, wherein the polypeptide is obtainable by the method of the eleventh aspect of the invention, wherein the heavy chain variable domain in the method is a human heavy chain variable domain and the light chain variable domain is from a non-human mammal. Preferably, the light chain variable domain of the antibody polypeptide is from a Camelid. Preferably, the light chain variable domain of the antibody polypeptide is provided as part of a Camelid IgG or an IgG derived from a Camelid. Preferably, the light chain variable domain of the antibody polypeptide is provided as part of a human IgG or an IgG derived from a human, and wherein the light chain variable domain is paired in the IgG with a heavy chain variable domain that is different from the heavy chain variable domain used in the eleventh aspect of the invention. The invention also provides a derivative of an antibody polypeptide according to the twelfth aspect, wherein the derivative has a CDR3 mutation as compared to the CDR3 of the variable domain of the polypeptide of the twelfth aspect. The invention also provides a derivative produced by affinity maturation of an antibody polypeptide of the twelfth aspect.

In one aspect, the invention provides a polypeptide comprising a half-life extending moiety linked to an antibody polypeptide of the 10^{th} or 12^{th} aspect or a derivative of, wherein the moiety is selected from a PEG; an antibody constant domain; an antibody Fc region; albumin or a fragment thereof; a peptide or an antibody fragment that binds albumin, an albumin fragment; the neonatal Fc receptor; transferring; or the transferring receptor. Preferably, polypeptide is a medicament, or for therapy and/or prevention of a disease or condition in a human. Preferably, the variable domain of the antibody polypeptide binds a target ligand selected from the group consisting of TNF alpha, serum albumin, von Willebrand's factor (vWF), IgE, interferon gamma, EGFR, IgE, MMP12, PDK1 and Amyloid beta (A-beta), or any one of the targets listed in Annex 1.

In one embodiment of a method according to the invention, the method further comprises the step of producing a mutant or derivative of the selected variable domain.

In one embodiment of a method according to the invention wherein a B-cell population is used, the B-cell population is provided in a plurality of wells or receptacles, wherein each well or receptacle contains a single B-cell type.

In one embodiment of a method according to the invention wherein a B-cell population is used, the B-cell population is provided in a plurality of wells or receptacles, wherein each well or receptacle contains on average one B-cell type.

In one aspect of the invention, there is provided a method for separating IgG from an antibody single variable domain in a population of antibody polypeptides comprising single variable domains and IgG, the method comprising:
a) contacting the population with a generic ligand and
b) selecting a subpopulation that binds to the generic ligand, thereby separating IgG from the single variable domain,
   wherein the generic ligand has binding specificity for antibody CH1 domain, light chain constant domain (CL), IgG hinge or antibody light chain variable domain.

Preferably, the generic ligand is selected from protein L, a protein L domain, or a derivative of protein L that binds light chain variable domain; protein G, a domain of protein G, or a derivative of protein G that binds CH1; an antibody and an antibody fragment, an affibody, an LDL receptor domain and an EGF domain.

Preferably, the generic ligand is selected from a dAb, Nanobody™, scFv, Fab, Fab′, F(ab)₂, F(ab′)₂, scFv, Fv or a disulphide bonded Fv.

Preferably, the variable domain is a human single variable domain and the IgG is human IgG.

Preferably, the generic ligand binds antibody CH1 domain, light chain constant domain (CL), IgG hinge or antibody light chain variable domain with an affinity of 1 mM or less.

Preferably, the generic ligand binds antibody CH1 domain, light chain constant domain (CL), IgG hinge or antibody light chain variable domain with an affinity of 1 micromolar or less.

Preferably, the generic ligand binds antibody CH1 domain, light chain constant domain (CL), IgG hinge or antibody light chain variable domain with an affinity of 100 nM or less.

In a thirteenth aspect, the invention provides a method for separating a Camelid VHH single variable domain from IgG in a population of antibody polypeptides comprising Camelid VHH domains and IgG, the method comprising:
a) contacting the population with a generic ligand and
b) selecting a subpopulation that binds to the generic ligand, thereby separating the single variable domain from IgG.

Preferably, the generic ligand is selected from an antibody light chain variable domain, an antibody and an antibody fragment.

Preferably, the generic ligand is selected from a dAb, Nanobody™, scFv, Fab, Fab′, F(ab)₂, F(ab′)₂, scFv, Fv or a disulphide bonded Fv.

Preferably, the generic ligand binds VHH or heavy chain antibody (H2) hinge with an affinity of 1 mM or less.

Preferably, the generic ligand binds VHH or heavy chain antibody (H2) hinge with an affinity of 1 micromolar or less.

Preferably, the generic ligand binds VHH or heavy chain antibody (H2) hinge with an affinity of 100 nM or less.

Preferably, the antibody polypeptide population is provided by B cells.

Preferably, the variable domain is Camelid VHH and the IgG is Camelid IgG.

Preferably, the variable domain is provided by a Camelid heavy chain (H2) antibody.

Preferably, the generic ligand is labelled or tagged.

In one embodiment of the method, antibody polypeptide, derivative or use of the invention, the generic ligand is an antibody variable domain selected from Annex 2 a), c), d) or e).

In a fourteenth aspect, the invention provides a method for selecting, from a repertoire of antibody polypeptides, a single variable domain which binds a target ligand and a generic ligand, comprising the steps of:
a) contacting the repertoire with a target ligand and selecting single variable domains bound thereto; and
b) contacting the selected variable domains with the generic ligand and selecting a variable domain which binds to the generic ligand,
wherein the generic ligand is an antibody variable domain selected from Annex 2 (c) or (e); and
wherein (i) when the selected variable domain is a heavy chain variable domain the generic ligand is a light chain variable domain, or (ii) when the selected variable domain is a light chain variable domain the generic ligand is a heavy chain variable domain.

Preferably, the repertoire of antibody polypeptides is a repertoire of heavy chain variable domains the generic ligand is a light chain variable domain.

Preferably, the repertoire of antibody polypeptides is a repertoire of light chain variable domains the generic ligand is a heavy chain variable domain.

Preferably, the method comprises the step of producing a mutant or derivative of the selected variable domain.

Preferably, the generic ligand binds the same target ligand species as the selected variable domain.

Preferably, the generic ligand binds a different target ligand species to the selected variable domain.

Preferably, the method comprises the step of combining the selected variable domain with an antibody variable domain that is identical to the generic ligand or a derivative thereof to produce a product with target ligand binding specificity.

In a fifteenth aspect, the invention provides a method of producing a derivative of an antibody or antibody fragment in any of Annex 2(c) (i) to (iv) that binds a target ligand, the method comprising:
a) using a heavy chain variable domain of said antibody or fragment or an identical variable domain as the generic ligand in the method of the 14^{th} aspect, and wherein the target ligand used in step a) is the target ligand to which the antibody or fragment binds, thereby selecting a light chains single variable domain that binds the target ligand and the heavy chain variable domain; and
b) replacing at least one of the light chain variable domains of the antibody or fragment with the selected light chain variable domain; an identical light chain variable domain or a derivative thereof.

In a sixteenth aspect, the invention provides a method of producing multispecific derivative of an antibody or antibody fragment in any of Annex 2 (c) (i) to (iv), the method comprising:
a) using heavy chain variable domain of said antibody or fragment (or an identical variable domain) as the generic ligand in the method of the 14^{th} aspect, and wherein the target ligand used in step a) is a target ligand that is different from the target ligand to which the antibody or fragment binds, thereby selecting a light chain single variable domain that binds the different target ligand and the heavy chain variable domain; and
b) replacing at least one of the light chain variable domains of the antibody or fragment with the selected light chain variable domain; an identical light chain variable domain or a derivative thereof, thereby producing a multispecific product.

Preferably, in the 15^{th} and 16^{th} aspects, the light chain variable domain is selected from a human light chain variable domain; a light chain variable domain derived from a human; a light chain variable domain having a FW2 sequence that is identical to FW2 encoded by germline gene sequence DPK9; a Camelid light chain variable domain; a light chain variable domain derived from a Camelid; and a humanised Camelid or murine light chain variable domain.

In a seventeenth aspect, the invention provides a method of producing a derivative of an antibody or antibody fragment in any of Annex 2(c) (i) to (iv) that binds a target ligand, the method comprising:
a) using a light chain variable domain of said antibody or fragment (or an identical variable domain) as the generic ligand in the method of the 14^{th} aspect, and wherein the target ligand used in step a) is the target ligand to which the antibody or fragment binds, thereby selecting a heavy chain single variable domain that binds the target ligand and the light chain variable domain; and
b) replacing at least one of the heavy chain variable domains of the antibody or fragment with the selected heavy chain variable domain; an identical heavy chain variable domain or a derivative thereof.

In an eighteenth aspect, the invention provides a method of producing multispecific derivative of a antibody or antibody fragment in any of Annex 2(c) (i) to (iv), the method comprising:
a) using a light chain variable domain of from said antibody or fragment or an identical variable domain as the generic ligand in the method of claim 94, and wherein the target ligand used in step a) is a target ligand that is different from the target ligand to which the antibody or fragment binds, thereby selecting a heavy chain single variable domain that binds the different target ligand and the light chain variable domain; and
b) replacing at least one of the heavy chain variable domains of the antibody or fragment with the selected heavy chain variable domain; an identical heavy chain variable domain or a derivative thereof, thereby producing a multispecific product.

Preferably, in the 17^{th} and 18^{th} aspects, the heavy chain variable domain is selected from is from the group consisting of heavy chain variable domains derived from a Camelid; a VHH domain; a Nanobody™; a VHH having a glycine at position 44; a VHH having a leucine at position 45; a VHH having a tryptophan at position 47; a VHH having a glycine at position 44 and a leucine at position 45; a VHH having a glycine at position 44 and a tryptophan at position 47; a VHH having a leucine at position 45 and a tryptophan at position 47; a VHH having a glycine at position 44, a leucine at position 45 and a tryptophan at position 47; a VHH having a tryptophan or arginine at position 103; a humanised Camelid or murine heavy chain variable domain; a humanised Nanobody™; a human heavy chain variable domain; a heavy chain variable domain derived from a human; a heavy chain variable domain having a FW2 sequence that is identical to FW2 encoded by germline gene sequence DP47; or a heavy chain variable domain having positions 44, 45 and 47 that are identical to positions 44, 45 and 47 encoded by germline gene sequence DP47.

Preferably, in the 15^{th} to 18^{th} aspects, either a) the selected variable domain is a heavy chain variable domain and each heavy chain variable domain of the antibody or fragment is replaced with the selected heavy chain variable domain; an identical heavy chain variable domain or a derivative thereof; or b) the selected variable domain is a light chain variable domain and each light chain variable domain of the antibody or fragment is replaced with the selected light chain variable domain; an identical light chain variable domain or a derivative thereof.

The invention also provides a derivative obtainable by the method of any one of the 15^{th} to 18^{th} aspects.

In a nineteenth aspect, the invention provides a derivative of an antibody or an antibody fragment selected from Panorex™, Rituxin™, Zevalin™, Mylotarg™, Campath™, Herceptin™, ReoPro™, Synagis™, Xolair™, Remicade™, Simulect™, OKT3™, Orthoclone™, Zenapax™, Humira™, Bexxar™, Raptiva™, Antegren™, Erbitux™ and Avastin™, obtainable by any one of the fifteenth to eighteenth aspects of the invention.

In a twentieth aspect, the invention provides the use of the derivative or product for therapy and/or prevention of a disease or condition in a human.

In a twenty-first aspect, the invention provides the use of the derivative or product for therapy and/or prevention of a disease or condition in a human.

In a twenty-second aspect, the invention provides the use of the derivative or product for therapy and/or prevention of a disease or condition in a human, wherein the condition is a condition listed for the antibody or antibody fragment in Annex 2(c) (i).

Where the invention involves the use of a VH or VHH generic ligand for selecting a VL domain or vice versa, the selection may be facilitated by the inherent pairing of such domains via interface regions. For example, relevant interface regions may comprise positions 44, 45 and 47 of VH and VHH domains (and the equivalent regions of VL domains), such numbering according to Kabat. Thus, the use of a VL with a human interface (e.g., 44G, 45L and 47W) may be useful to select Camelid VHH domains from a library, since this may select VHH domains having interface regions that pair well with human VL interface regions. This, therefore, selects for human-like features in VHH domains, thus providing useful VHH products for us in humans (e.g. for therapy of diseases) and/or useful leads for further development of products compatible for use in humans.

### BRIEF DESCRIPTION OF THE FIGURES

FIG. 1: Bar graph indicating positions in the VH and V_{κ} regions of the human antibody repertoire which exhibit extensive natural diversity and make antigen contacts (see Tomlinson et al. (1996) J. Mol. Biol., 256: 813). The H3 and the end of L3 are not shown in this representation although they are also highly diverse and make antigen contacts. Although sequence diversity in the human lambda genes has been thoroughly characterised (see Ignatovich et al. (1997) J. Mol. Biol, 268: 69) very little data on antigen contacts currently exists for three-dimensional lambda structures.

FIG. 2: Sequence of the scFv that forms the basis of a library according to the invention. There are currently two versions of the library: a “primary” library wherein 18 positions are varied and a “somatic” library wherein 12 positions are varied. The six loop regions H1, H2, H3, L1, L2 and L3 are indicated. CDR regions as defined by Kabat (Kabat et al. (1991). Sequences of proteins of immunological interest, U.S. Department of Health and Human Services) are underlined.

FIG. 3: Analysis of functionality in a library according to the invention before and after selecting with the generic ligands Protein A and Protein L. Here Protein L is coated on an ELISA plate, the scFv supernatants are bound to it and detection of scFv binding is with Protein A-HRP. Therefore, only those scFv capable of binding both Protein A and Protein L give an ELISA signal.

FIG. 4: Sequences of clones selected from libraries according to the invention, after panning with bovine ubiquitin, rat BIP, bovine histone, NIP-BSA, FITC-BSA, human leptin, human thyroglobulin, BSA, hen egg lysozyme, mouse IgG and human IgG. Underlines in the sequences indicate the positions which were varied in the respective libraries.

FIG. 5: 5a: Comparison of scFv concentration produced by the unselected and preselected “primary” DVT libraries in host cells. 5b: standard curve of ELISA as determined from known standards.

FIG. 6: Western blot of phage from preselected and unselected DVT “primary” libraries, probed with an anti-phage pIII antibody in order to determine the percentage of phage bearing scFv.

### DETAILED DESCRIPTION OF THE INVENTION

### Definitions

Repertoire A repertoire is a population of diverse variants, for example nucleic acid variants which differ in nucleotide sequence or polypeptide variants which differ in amino acid sequence. A library according to the invention will encompass a repertoire of polypeptides or nucleic acids. According to the present invention, a repertoire of polypeptides is designed to possess a binding site for a generic ligand and a binding site for a target ligand. The binding sites may overlap, or be located in the same region of the molecule, but their specificities will differ.

Organism As used herein, the term “organism” refers to all cellular life-forms, such as prokaryotes and eukaryotes, as well as non-cellular, nucleic acid-containing entities, such as bacteriophage and viruses.

Functional As used herein, the term “functional” refers to a polypeptide which possesses either the native biological activity of the naturally-produced proteins of its type, or any specific desired activity, for example as judged by its ability to bind to ligand molecules, defined below. Examples of “functional” polypeptides include an antibody binding specifically to an antigen through its antigen-binding site, a receptor molecule (e.g. a T-cell receptor) binding its characteristic ligand and an enzyme binding to its substrate. In order for a polypeptide to be classified as functional according to the invention, it follows that it first must be properly processed and folded so as to retain its overall structural integrity, as judged by its ability to bind the generic ligand, also defined below.

For the avoidance of doubt, functionality is not equivalent to the ability to bind the target ligand. For instance, a functional anti-CEA monoclonal antibody will not be able to bind specifically to target ligands such as bacterial LPS. However, because it is capable of binding a target ligand (i.e. it would be able bind to CEA if CEA were the target ligand) it is classed as a “functional” antibody molecule and may be selected by binding to a generic ligand, as defined below. Typically, non-functional antibody molecules will be incapable of binding to any target ligand.

Generic ligand A generic ligand is a ligand that binds a substantial proportion of functional members in a given repertoire. Thus, the same generic ligand can bind many members of the repertoire regardless of their target ligand specificities (see below). In general, the presence of functional generic ligand binding site indicates that the repertoire member is expressed and folded correctly. Thus, binding of the generic ligand to its binding site provides a method for preselecting functional polypeptides from a repertoire of polypeptides.

Target Ligand The target ligand is a ligand for which a specific binding member or members of the repertoire is to be identified. Where the members of the repertoire are antibody molecules, the target ligand may be an antigen and where the members of the repertoire are enzymes, the target ligand may be a substrate. Binding to the target ligand is dependent upon both the member of the repertoire being functional, as described above under generic ligand, and upon the precise specificity of the binding site for the target ligand.

Subset The subset is a part of the repertoire. In the terms of the present invention, it is often the case that only a subset of the repertoire is functional and therefore possesses a functional generic ligand binding site. Furthermore, it is also possible that only a fraction of the functional members of a repertoire (yet significantly more than would bind a given target ligand) will bind the generic ligand. These subsets are able to be selected according to the invention.

Subsets of a library may be combined or pooled to produce novel repertoires which have been preselected according to desired criteria. Combined or pooled repertoires may be simple mixtures of the polypeptide members preselected by generic ligand binding, or may be manipulated to combine two polypeptide subsets. For example, VH and VL polypeptides may be individually prescreened, and subsequently combined at the genetic level onto single vectors such that they are expressed as combined VH-VL dimers, such as scFv.

Library The term library refers to a mixture of heterogeneous polypeptides or nucleic acids. The library is composed of members, which have a single polypeptide or nucleic acid sequence. To this extent, library is synonymous with repertoire. Sequence differences between library members are responsible for the diversity present in the library. The library may take the form of a simple mixture of polypeptides or nucleic acids, or may be in the form organisms or cells, for example bacteria, viruses, animal or plant cells and the like, transformed with a library of nucleic acids. Preferably, each individual organism or cell contains only one member of the library. Advantageously, the nucleic acids are incorporated into expression vectors, in order to allow expression of the polypeptides encoded by the nucleic acids. In a preferred aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

Immunoglobulin superfamily This refers to a family of polypeptides which retain the immunoglobulin fold characteristic of immunoglobulin (antibody) molecules, which contains two β sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor). The present invention is applicable to all immunoglobulin superfamily molecules, since variation therein is achieved in similar ways. Preferably, the present invention relates to immunoglobulins (antibodies).

Main-chain conformation The main-chain conformation refers to the C□ backbone trace of a structure in three-dimensions. When individual hypervariable loops of antibodies or TCR molecules are considered the main-chain conformation is synonymous with the canonical structure. As set forth in Chothia and Lesk (1987) J. Mol. Biol., 196: 901 and Chothia et al. (1989) Nature, 342: 877, antibodies display a limited number of canonical structures for five of their six hypervariable loops (H1, H2, L1, L2 and L3), despite considerable side-chain diversity in the loops themselves. The precise canonical structure exhibited depends on the length of the loop and the identity of certain key residues involved in its packing. The sixth loop (H3) is much more diverse in both length and sequence and therefore only exhibits canonical structures for certain short loop lengths (Martin et al. (1996) J. Mol. Biol., 263: 800; Shirai et al (1996) FEBS Letters, 399: 1). In the present invention, all six loops will preferably have canonical structures and hence the main-chain conformation for the entire antibody molecule will be known.

Antibody polypeptide Antibodies are immunoglobulins that are produced by B cells and form a central part of the host immune defence system in vertebrates. An antibody polypeptide, as used herein, is a polypeptide which either is an antibody or is a part of an antibody, modified or unmodified. Thus, the term antibody polypeptide includes a heavy chain, a light chain, a heavy chain-light chain dimer, a Fab fragment, a F (ab′)₂ fragment, a Dab fragment, or an Fv fragment, including a single chain Fv (scFv). Methods for the construction of such antibody molecules are well known in the art.

Superantigen Superantigens are antigens, mostly in the form of toxins expressed in bacteria, which interact with members of the immunoglobulin superfamily outside the conventional ligand binding sites for these molecules. Staphylococcal enterotoxins interact with T-cell receptors and have the effect of stimulating CD4+ T-cells. Superantigens for antibodies include the molecules Protein G that binds the IgG constant region (Bjorck and Kronvall (1984) J. Immunol, 133: 969; Reis et al. (1984) J. Immunol., 132: 3091), Protein A that binds the IgG constant region and the VH domain (Forsgren and Sjoquist (1966) J. Immunol., 97: 822) and Protein L that binds the VL domain (Bjorck (1988) J. Immunol., 140: 1994).

### Preferred Embodiments of the Invention

The present invention provides a selection system which eliminates (or significantly reduces the proportion of) non-functional or poorly folded/expressed members of a polypeptide library whilst enriching for functional, folded and well expressed members before a selection for specificity against a “target ligand” is carried out. A repertoire of polypeptide molecules is contacted with a “generic ligand”, a protein that has affinity for a structural feature common to all functional, for example complete and/or correctly folded, proteins of the relevant class. Note that the term “ligand” is used broadly in reference to molecules of use in the present invention. As used herein, the term “ligand” refers to any entity that will bind to or be bound by a member of the polypeptide library.

A significant number of defective proteins present in the initial repertoire fail to bind the generic ligand and are thereby eliminated. This selective removal of non-functional polypeptides from a library results in a marked reduction in its actual size, while its functional size is maintained, with a corresponding increase in its quality. Polypeptides which are retained by virtue of binding the generic ligand constitute a ‘first selected pool’ or ‘subset’ of the original repertoire. Consequently, this ‘subset’ is enriched for functional, well folded and well expressed members of the initial repertoire.

The polypeptides of the first selected pool or subset are subsequently contacted with at least one “target ligand”, which binds to polypeptides with a given functional specificity. Such target ligands include, but are not limited to, either half of a receptor/ligand pair (e.g. a hormone or other cell-signalling molecule, such as a neurotransmitter, and its cognate receptor), either of a binding pair of cell adhesion molecules, a protein substrate that is bound by the active site of an enzyme, a protein, peptide or small organic compound against which a particular antibody is to be directed or even an antibody itself. Consequently, the use of such a library is less labour-intensive and more economical, in terms of both time and materials, than is that of a conventional library. In addition, since, compared to a repertoire which has not been selected with a generic ligand, the first selected pool will contain a much higher ratio of molecules able to bind the target ligand to those that are unable to bind the target ligand, there will be a significant reduction of background during selection with the “target ligand”.

Combinatorial selection schemes are also contemplated according to the invention. Multiple selections of the same initial polypeptide repertoire can be performed in parallel or in series using different generic and/or target ligands. Thus, the repertoire can first be selected with a single generic ligand and then subsequently selected in parallel using different target ligands. The resulting subsets can then be used separately or combined, in which case the combined subset will have a range of target ligand specificities but a single generic ligand specificity. Alternatively, the repertoire can first be selected with a single target ligand and then subsequently selected in parallel using different generic ligands. The resulting subsets can then be used separately or combined, in which case the combined subset will have a range of generic ligand specificities but a single target ligand specificity. The use of more elaborate schemes are also envisaged. For example, the initial repertoire can be subjected to two rounds of selection using two different generic ligands, followed by selection with the target ligand. This produces a subset in which all members bind both generic ligands and the target ligand. Alternatively, if the selection of the initial repertoire with the two generic ligands is performed in parallel and the resulting subsets combined and then selected with the target ligand the resulting subset binds at least one of the two generic ligands and the target ligand. Combined or pooled repertoires may be simple mixtures of the subsets or may be manipulated to physically link the subsets. For example, VH and VL polypeptides may be individually selected in parallel by binding two different generic ligands, and subsequently combined at the genetic level onto single vectors such that they are expressed as combined VH-VL. This repertoire can then be selected against the target ligand such that the selected members able to bind both generic ligands and the target ligand.

The invention encompasses libraries of functional polypeptides selected or selectable by the methods broadly described above, as well as nucleic acid libraries encoding polypeptide molecules which may be used in a selection performed according to these methods (preferably, molecules which comprise a first binding site for a target ligand and a second binding site for a generic ligand). In addition, the invention provides methods for detecting, immobilising, purifying or immunoprecipitating one or more members of a repertoire of functional polypeptides selected using the generic or target ligands according to the invention.

The invention is particularly applicable to the enrichment of libraries of molecules of the immunoglobulin superfamily. This is particularly true as regards the generation of populations of antibodies and T-cell receptors which are functional and have a desired specificity, as is required for use in diagnostic, therapeutic or prophylactic procedures. To this end, the invention provides antibody and T-cell receptor libraries wherein all the members have both natural frameworks and loops of known main-chain conformation, as well as strategies for useful mutagenesis of the starting sequence and the subsequent selection of functional variants so generated. Such polypeptide libraries may comprise VH or V_{β} domains or, alternatively, it may comprise VL or V_{α} domains, or even both VH or V_{β} and VL or V_{α} domains.

There is significant need in the art for improved libraries of antibody or T-cell receptor molecules. For example, despite progress in the creation of “single pot” phage-antibody libraries, several problems still remain. Natural libraries (Marks et al. (1991) J. Mol. Biol., 222: 581; Vaughan et al. (1996) Nature Biotech., 14: 309) which use rearranged V genes harvested from human B cells are highly biased due to the positive and negative selection of the B cells in vivo. This can limit the effective size of phage libraries constructed from rearranged V genes. In addition, clones derived from natural libraries invariably contain framework mutations which may effect the antibodies immunogenicity when used in human therapy. Synthetic libraries (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97) can overcome the problem of bias but they require the use of long degenerate PCR primers which frequently introduce base-pair deletions into the assembled V genes. This high degree of randomisation may also lead to the creation of antibodies which are unable to fold correctly and are also therefore non-functional. In many cases it is likely that these non-functional members will outnumber the functional members in a library. Even if the frameworks can be pre-optimised for folding and/or expression (WO97/08320, Morphosys) by synthesising a set of ‘master genes’ with consensus framework sequences and by incorporating amino acid substitutions shown to improve folding and expression, there remains the problem of immunogenicity since, in most cases, the consensus sequences do not correspond to any natural framework. Furthermore, since it is likely that the CDR diversity will also have an effect of folding and/or expression, it is preferable to optimise the folding and/or expression (and remove any frame-shifts or stop codons) after the V gene has been fully assembled.

A further problem with existing libraries is that because the main-chain conformation is heterogeneous, three-dimensional structural modelling is difficult because suitable high resolution crystallographic data may not be available. This is a particular problem for the H3 region, where the vast majority of antibodies derived from natural or synthetic antibody libraries have medium length or long loops and therefore cannot be modelled.

Another problem with existing libraries is the reliance on epitope tags (such as the myc, FLAG or HIS tags) for detection of expressed antibody fragments. As these are usually located at the N or C terminal ends of the antibody fragment they tend to be prone to proteolytic cleavage. Superantigens, such as Protein A and Protein L can be used to detect expressed antibody fragments by binding the folded domains themselves but since they are VH and VL family specific, only a relatively small proportion of members of any existing antibody library will bind one of these reagents and an even smaller proportion will bind to both.

To this end, it would be desirable to have a selection system which could eliminate (or at least reduce the proportion of) non-functional or poorly folded/expressed members of the library before selection against the target antigen is carried out whilst enriching for functional, folded and well expressed members all of which are able to bind generic ligands such as the superantigens Protein A and Protein L. In addition, it would be advantageous to construct an antibody library wherein all the members have natural frameworks and have loops with known main-chain conformations.

The invention accordingly provides a method by which a polypeptide repertoire may be selected to remove non-functional members. This results in a marked reduction in the actual library size (and a corresponding increase in the quality of the library) without reducing the functional library size. The invention also provides a method for creating new polypeptide repertoires wherein all the functional members are able to bind a given generic ligand. The same generic ligand can be used for the subsequent detection, immobilisation, purification or immunoprecipitation of any one or more members of the repertoire.

Any ‘naïve’ or ‘immune’ antibody repertoire can be used with the present invention to enrich for functional members and/or to enrich for members that bind a given generic ligand or ligands. Indeed, since only a small percentage of all human germline VH segments bind Protein A with high affinity and only a small percentage of all human germline VL segments bind Protein L with high affinity preselection with these superantigens is highly advantageous. Alternatively, pre-selection with via the epitope tag enables non-functional variants to be removed from synthetic libraries. The libraries that are amenable to preselection include, but are not limited to, libraries comprised of V genes rearranged in vivo of the type described by Marks et al. (1991) J. Mol. Biol., 222: 581 and Vaughan et al. (1996) Nature Biotech., 14: 309, synthetic libraries whereby germline V gene segments are ‘rearranged’ in vitro (Hoogenboom & Winter (1992) J. Mol. Biol., 227: 381; Nissim et al. (1994) EMBO J., 13: 692; Griffiths et al. (1994) EMBO J., 13: 3245; De Kruif et al. (1995) J. Mol. Biol., 248: 97) or where synthetic CDRs are incorporated into a single rearranged V gene (Barbas et al. (1992) Proc. Natl. Acad. Sci. USA, 89: 4457) or into multiple master frameworks (WO97/08320, Morphosys).

### Selection of Polypeptides According to the Invention

Once a diverse pool of polypeptides is generated, selection according to the invention is applied. Two broad selection procedures are based upon the order in which the generic and target ligands are applied; combinatorial variations on these schemes involve the use of multiple generic and/or target ligands in a given step of a selection. When a combinatorial scheme is used, the pool of polypeptide molecules may be contacted with, for example, several target ligands at once, or by each singularly, in series; in the latter case, the resulting selected pools of polypeptides may be kept separate or may, themselves, be pooled. These selection schemes may be summarized as follows:

a. Selection Procedure 1:

### Initial Polypeptide Selection Using the Generic Ligand

In order to remove non-functional members of the library, a generic ligand is selected, such that the generic ligand is only bound by functional molecules. For example, the generic ligand may be a metallic ion, an antibody (in the form of a monoclonal antibody or a polyclonal mixture of antibodies), half of an enzyme/ligand complex or organic material; note that ligands of any of these types are, additionally or alternatively, of use as target ligands according to the invention. Antibody production and metal affinity chromatography are discussed in detail below. Ideally, these ligands bind a site (e.g. a peptide tag or superantigen binding site) on the members of the library which is of constant structure or sequence, which structure is liable to be absent or altered in non-functional members. In the case of antibody libraries, this method is of use to select from a library only those functional members which have a binding site for a given superantigen or monoclonal antibody; such an approach is useful in selecting functional antibody polypeptides from both natural and synthetic pools thereof.

The superantigens Protein A and/or Protein L are of use in the invention as generic ligands to select antibody repertoires, since they bind correctly folded VH and VL domains (which belong to certain VH and VL families), respectively, regardless of the sequence and structure of the binding site for the target ligand. In addition, Protein A or another superantigen Protein G are of use as generic ligands to select for folding and/or expression by binding the heavy chain constant domains of antibodies. Anti-κ and anti-λ antibodies are also of use in selecting light chain constant domains. Small organic mimetics of antibodies or of other binding proteins, such as Protein A (Li et al. (1998) Nature Biotech., 16: 190), are also of use.

When this selection procedure is used, the generic ligand, by its very nature, is able to bind all functional members of the preselected repertoire; therefore, this generic ligand (or some conjugate thereof) may be used to detect, immobilise, purify or immunoprecipitate any member or population of members from the repertoire (whether selected by binding a given target ligand or not, as discussed below). Protein detection via immunoassay techniques as well as immunoprecipitation of member polypeptides of a repertoire of the invention may be performed by the techniques discussed below with regard to the testing of antibody selection ligands of use in the invention (see “Antibodies for use as ligands in polypeptide selection”). Immobilization may be performed through specific binding of a polypeptide member of a repertoire to either a generic or target ligand according to the invention which is, itself, linked to a solid or semi-solid support, such as a filter (e.g. of nitrocellulose or nylon) or a chromatographic support (including, but not limited to, a cellulose, polymer, resin or silica support); covalent attachment of the member polypeptide to the generic or target ligand may be performed using any of a number of chemical crosslinking agents known to one of skill in the art. Immobilization on a metal affinity chromatography support is described below (see “Metallic ligands as use for the selection of polypeptides”). Purification may comprise any or a combination of these techniques, in particular immunoprecipitation and chromatography by methods well known in the art.

Using this approach, selection with multiple generic ligands can be performed either one after another to create a repertoire in which all members bind two or more generic ligands, separately in parallel, such that the subsets can then be combined (in this case, members of the preselected repertoire will bind at least one of the generic ligands) or separately followed by incorporation into the same polypeptide chain whereby a large functional library in which all members may be able to bind all the generic ligands used during preselection. For example, subsets can be selected from one or more libraries using different generic ligands which bind heavy and light chains of antibody molecules (see below) and then combined to form a heavy/light chain library, in which the heavy and light chains are either non-covalently associated or are covalently linked, for example, by using VH and VL domains in a single-chain Fv context.

### Secondary Polypeptide Selection Using the Target Ligand

Following the selection step with the generic ligand, the library is screened in order to identify members that bind to the target ligand. Since it is enriched for functional polypeptides after selection with the generic ligand, there will be an advantageous reduction in non-specific (“background”) binding during selection with the target ligand. Furthermore, since selection with the generic ligand produces a the marked reduction in the actual library size (and a corresponding increase in the quality of the library) without reducing the functional library size, a smaller repertoire should elicit the same diversity of target ligand specifities and affinities as the larger starting repertoire (that contained many non-functional and poorly folded/expressed members).

One or more target ligands may be used to select polypeptides from the first selected polypeptide pool generated using the generic ligand. In the event that two or more target ligands are used to generate a number of different subsets, two or more of these subsets may be combined to form a single, more complex subset. A single generic ligand is able to bind every member of the resulting combined subset; however, a given target ligand binds only a subset of library members.

b. Selection Procedure 2:
Initial Selection of Repertoire Members with the Target Ligand

Here, selection using the target ligand is performed prior to selection using the generic ligand. Obviously, the same set of polypeptides can result from either scheme, if such a result is desired. Using this approach, selection with multiple target ligands can be performed in parallel or by mixing the target ligands for selection. If performed in parallel, the resulting subsets may, if required, be combined.

### Secondary Polypeptide Selection Using the Generic Ligand

Subsequent selection of the target ligand binding subset can then be performed using one or more generic ligands. Whilst this is not a selection for function, since members of the repertoire that are able to bind to the target ligand are by definition functional, it does enable subsets that bind to different generic ligands to be isolated. Thus, the target ligand selected population can be selected by one generic ligand or by two or more generic ligands. In this case, the generic ligands can be used one after another to create a repertoire in which all members bind the target ligand and two or more generic ligands or separately in parallel, such that different (but possibly overlapping) subsets binding the target ligand and different generic ligands are created. These can then be combined (in this case, members will bind at least one of the generic ligands).

### Selection of Immunoglobulin-Family Polypeptide Library Members

The members of the repertoires or libraries selected in the present invention advantageously belong to the immunoglobulin superfamily of molecules, in particular, antibody polypeptides or T-cell receptor polypeptides. For antibodies, it is envisaged that the method according to this invention may be applied to any of the existing antibody libraries known in the art (whether natural or synthetic) or to antibody libraries designed specifically to be preselected with generic ligands (see below).

### Construction of Libraries of the Invention

a. Selection of the Main-Chain Conformation

The members of the immunoglobulin superfamily all share a similar fold for their polypeptide chain. For example, although antibodies are highly diverse in terms of their primary sequence, comparison of sequences and crystallographic structures has revealed that, contrary to expectation, five of the six antigen binding loops of antibodies (H1, H2, L1, L2, and L3) adopt a limited number of main-chain conformations, or canonical structures (Chothia and Lesk (1987) supra; Chothia et al (1989) supra). Analysis of loop lengths and key residues has therefore enabled prediction of the main-chain conformations of H1, H2, L1, L2 and L3 found in the majority of human antibodies (Chothia et al. (1992) supra; Tomlinson et al. (1995) supra; Williams et al. (1996) supra). Although the H3 region, is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) supra; Shirai et al. (1996) supra).

According to the present invention, libraries of antibody polypeptides are designed in which certain loop lengths and key residues have been chosen to ensure that the main-chain conformation of the members is known. Advantageously, these are real conformations of immunoglobulin superfamily molecules found in nature, to minimize the chances that they are non-functional, as discussed above. Germline V gene segments serve as one suitable basic framework for constructing antibody or T-cell receptor libraries; other sequences are also of use. Variations may occur at a low frequency, such that a small number of functional members may possess an altered main-chain conformation, which does not affect its function.

Canonical structure theory is also of use in the invention to assess the number of different main-chain conformations encoded by antibodies, to predict the main-chain conformation based on antibody sequences and to choose residues for diversification which do not affect the canonical structure. It is now known that, in the human V_{κ} domain, the L1 loop can adopt one of four canonical structures, the L2 loop has a single canonical structure and that 90% of human V_{κ} domains adopt one of four or five canonical structures for the L3 loop (Tomlinson et al. (1995) supra); thus, in the V_{κ} domain alone, different canonical structures can combine to create a range of different main-chain conformations. Given that the V_{λ} domain encodes a different range of canonical structures for the L1, L2 and L3 loops and that V_{κ} and V_{λ} domains can pair with any VH domain which can encode several canonical structures for the H1 and H2 loops, the number of canonical structure combinations observed for these five loops is very large. This implies that the generation of diversity in the main-chain conformation may be essential for the production of a wide range of binding specificities. However, by constructing an antibody library based on a single known main-chain conformation it was found, contrary to expectation, that diversity in the main-chain conformation is not required to generate sufficient diversity to target substantially all antigens. Even more surprisingly, the single main-chain conformation need not be a consensus structure—a single naturally occurring conformation can be used as the basis for an entire library. Thus, in a preferred aspect, the invention provides a library in which the members encode a single known main-chain conformation. It is to be understood, however, that occasional variations may occur such that a small number of functional members may possess an alternative main-chain conformation, which may be unknown.

The single main-chain conformation that is chosen is preferably commonplace among molecules of the immunoglobulin superfamily type in question. A conformation is commonplace when a significant number of naturally occurring molecules are observed to adopt it. Accordingly, in a preferred aspect of the invention, the natural occurrence of the different main-chain conformations for each binding loop of an immunoglobulin superfamily molecule are considered separately and then a naturally occurring immunoglobulin superfamily molecule is chosen which possesses the desired combination of main-chain conformations for the different loops. If none is available, the nearest equivalent may be chosen. Since a disadvantage of immunoglobulin-family polypeptide libraries of the prior art is that many members have unnatural frameworks or contain framework mutations (see above), in the case of antibodies or T-cell receptors, it is preferable that the desired combination of main-chain conformations for the different loops is created by selecting germline gene segments which encode the desired main-chain conformations. It is more preferable, that the selected germline gene segments are frequently expressed and most preferable that they are the most frequently expressed.

In designing antibody libraries, therefore, the incidence of the different main-chain conformations for each of the six antigen binding loops may be considered separately. For H1, H2, L1, L2 and L3, a given conformation that is adopted by between 20% and 100% of the antigen binding loops of naturally occurring molecules is chosen. Typically, its observed incidence is above 35% (i.e. between 35% and 100%) and, ideally, above 50% or even above 65%. Since the vast majority of H3 loops do not have canonical structures, it is preferable to select a main-chain conformation which is commonplace among those loops which do display canonical structures. For each of the loops, the conformation which is observed most often in the natural repertoire is therefore selected. In human antibodies, the most popular canonical structures (CS) for each loop are as follows: H1-CS 1 (79% of the expressed repertoire), H2-CS 3 (46%), L1-CS 2 of V_{κ} (39%), L2-CS 1 (100%), L3-CS 1 of V_{κ} (36%) calculation assumes a κ:λ ratio of 70:30, Hood et al. (1967) Cold Spring Harbor Symp. Quant. Biol., 48: 133). For H3 loops that have canonical structures, a CDR3 length (Kabat et al. (1991) Sequences of proteins of immunological interest, U.S. Department of Health and Human Services) of seven residues with a salt-bridge from residue 94 to residue 101 appears to be the most common. There are at least 16 human antibody sequences in the EMBL data library with the required H3 length and key residues to form this conformation and at least two crystallographic structures in the protein data bank which can be used as a basis for antibody modelling (2cgr and 1tet). The most frequently expressed germline gene segments that this combination of canonical structures are the VH segment 3-23 (DP-47), the J_{H} segment JH4b, the V_{κ} segment O2/O12 (DPK9) and the J_{κ} segment J_{κ} 1. These segments can therefore be used in combination as a basis to construct a library with the desired single main-chain conformation.

Alternatively, instead of choosing the single main-chain conformation based on the natural occurrence of the different main-chain conformations for each of the binding loops in isolation, the natural occurrence of combinations of main-chain conformations is used as the basis for choosing the single main-chain conformation. In the case of antibodies, for example, the natural occurrence of canonical structure combinations for any two, three, four, five or for all six of the antigen binding loops can be determined. Here, it is preferable that the chosen conformation is commonplace in naturally occurring antibodies and most preferable that it observed most frequently in the natural repertoire. Thus, in human antibodies, for example, when natural combinations of the five antigen binding loops, H1, H2, L1, L2 and L3, are considered, the most frequent combination of canonical structures is determined and then combined with the most popular conformation for the H3 loop, as a basis for choosing the single main-chain conformation.

b. Diversification of the Canonical Sequence

Having selected several known main-chain conformations or, preferably a single known main-chain conformation, the library of the invention is constructed by varying the binding site of the molecule in order to generate a repertoire with structural and/or functional diversity. This means that variants are generated such that they possess sufficient diversity in their structure and/or in their function so that they are capable of providing a range of activities. For example, where the polypeptides in question are cell-surface receptors, they may possess a diversity of target ligand binding specificities.

The desired diversity is typically generated by varying the selected molecule at one or more positions. The positions to be changed can be chosen at random or are preferably selected. The variation can then be achieved either by randomization, during which the resident amino acid is replaced by any amino acid or analogue thereof, natural or synthetic, producing a very large number of variants or by replacing the resident amino acid with one or more of a defined subset of amino acids, producing a more limited number of variants.

Various methods have been reported for introducing such diversity. Error-prone PCR (Hawkins et al. (1992) J. Mol. Biol., 226: 889), chemical mutagenesis (Deng et al. (1994) J. Biol. Chem., 269: 9533) or bacterial mutator strains (Low et al. (1996) J. Mol. Biol., 260: 359) can be used to introduce random mutations into the genes that encode the molecule. Methods for mutating selected positions are also well known in the art and include the use of mismatched oligonucleotides or degenerate oligonucleotides, with or without the use of PCR. For example, several synthetic antibody libraries have been created by targeting mutations to the antigen binding loops. The H3 region of a human tetanus toxoid-binding Fab has been randomized to create a range of new binding specificities (Barbas et al. (1992) supra). Random or semi-random H3 and L3 regions have been appended to germline V gene segments to produce large libraries with unmutated framework regions (Hoogenboom and Winter (1992) supra; Nissim et al. (1994) supra; Griffiths et al. (1994) supra; De Kruif et al. (1995) supra). Such diversification has been extended to include some or all of the other antigen binding loops (Crameri et al. (1996) Nature Med., 2: 100; Riechmann et al. (1995) Bio/Technology, 13: 475; Morphosys, WO97/08320, supra).

Since loop randomization has the potential to create approximately more than 10¹⁵ structures for H3 alone and a similarly large number of variants for the other five loops, it is not feasible using current transformation technology or even by using cell free systems to produce a library representing all possible combinations. For example, in one of the largest libraries constructed to date, 6×10¹⁰ different antibodies, which is only a fraction of the potential diversity for a library of this design, were generated (Griffiths et al. (1994) supra).

In addition to the removal of non-functional members and the use of a single known main-chain conformation, the present invention addresses these limitations by diversifying only those residues which are directly involved in creating or modifying the desired function of the molecule. For many molecules, the function will be to bind a target ligand and therefore diversity should be concentrated in the target ligand binding site, while avoiding changing residues which are crucial to the overall packing of the molecule or to maintaining the chosen main-chain conformation; therefore, the invention provides a library wherein the selected positions to be varied may be those that constitute the binding site for the target ligand.

### Diversification of the Canonical Sequence as it Applies to Antibodies

In the case of an antibody library, the binding site for the target ligand is most often the antigen binding site. Thus, in a highly preferred aspect, the invention provides an antibody library in which only those residues in the antigen binding site are varied. These residues are extremely diverse in the human antibody repertoire and are known to make contacts in high-resolution antibody/antigen complexes. For example, in L2 it is known that positions 50 and 53 are diverse in naturally occurring antibodies and are observed to make contact with the antigen. In contrast, the conventional approach would have been to diversify all the residues in the corresponding Complementarity Determining Region (CDR1) as defined by Kabat et al. (1991, supra), some seven residues compared to the two diversified in the library according to the invention. This represents a significant improvement in terms of the functional diversity required to create a range of antigen binding specificities.

In nature, antibody diversity is the result of two processes: somatic recombination of germline V, D and J gene segments to create a naive primary repertoire (so called germline and junctional diversity) and somatic hypermutation of the resulting rearranged V genes. Analysis of human antibody sequences has shown that diversity in the primary repertoire is focused at the centre of the antigen binding site whereas somatic hypermutation spreads diversity to regions at the periphery of the antigen binding site that are highly conserved in the primary repertoire (see Tomlinson et al. (1996) supra). This complementarity has probably evolved as an efficient strategy for searching sequence space and, although apparently unique to antibodies, it can easily be applied to other polypeptide repertoires according to the invention. According to the invention, the residues which are varied are a subset of those that form the binding site for the target ligand. Different (including overlapping) subsets of residues in the target ligand binding site are diversified at different stages during selection, if desired.

In the case of an antibody repertoire, the two-step process of the invention is analogous to the maturation of antibodies in the human immune system. An initial ‘naive’ repertoire is created where some, but not all, of the residues in the antigen binding site are diversified. As used herein in this context, the term “naive” refers to antibody molecules that have no pre-determined target ligand. These molecules resemble those which are encoded by the immunoglobulin genes of an individual who has not undergone immune diversification, as is the case with fetal and newborn individuals, whose immune systems have not yet been challenged by a wide variety of antigenic stimuli. This repertoire is then selected against a range of antigens. If required, further diversity can then be introduced outside the region diversified in the initial repertoire. This matured repertoire can be selected for modified function, specificity or affinity.

The invention provides two different naive repertoires of antibodies in which some or all of the residues in the antigen binding site are varied. The “primary” library mimics the natural primary repertoire, with diversity restricted to residues at the centre of the antigen binding site that are diverse in the germline V gene segments (germline diversity) or diversified during the recombination process (junctional diversity). Those residues, which are diversified include, but are not limited to, H50, H52, H52a, H53, H55, H56, H58, H95, H96, H97, H98, L50, L53, L91, L92, L93, L94 and L96. In the “somatic” library, diversity is restricted to residues that are diversified during the recombination process (junctional diversity) or are highly somatically mutated). Those residues which are diversified include, but are not limited to: H31, H33, H35, H95, H96, H97, H98, L30, L31, L32, L34 and L96. All the residues listed above as suitable for diversification in these libraries are known to make contacts in one or more antibody-antigen complexes. Since in both libraries, not all of the residues in the antigen binding site are varied, additional diversity is incorporated during selection by varying the remaining residues, if it is desired to do so. It shall be apparent to one skilled in the art that any subset of any of these residues (or additional residues which comprise the antigen binding site) can be used for the initial and/or subsequent diversification of the antigen binding site.

In the construction of libraries according to the invention, diversification of chosen positions is typically achieved at the nucleic acid level, by altering the coding sequence which specifies the sequence of the polypeptide such that a number of possible amino acids (all 20 or a subset thereof) can be incorporated at that position. Using the IUPAC nomenclature, the most versatile codon is NNK, which encodes all amino acids as well as the TAG stop codon. The NNK codon is preferably used in order to introduce the required diversity. Other codons which achieve the same ends are also of use, including the NNN codon, which leads to the production of the additional stop codons TGA and TAA.

A feature of side-chain diversity in the antigen binding site of human antibodies is a pronounced bias which favours certain amino acid residues. If the amino acid composition of the ten most diverse positions in each of the VH, V_{κ} and V_{λ} regions are summed, more than 76% of the side-chain diversity comes from only seven different residues, these being, serine (24%), tyrosine (14%), asparagine (11%), glycine (9%), alanine (7%), aspartate (6%) and threonine (6%). This bias towards hydrophilic residues and small residues which can provide main-chain flexibility probably reflects the evolution of surfaces which are predisposed to binding a wide range of antigens and may help to explain the required promiscuity of antibodies in the primary repertoire.

Since it is preferable to mimic this distribution of amino acids, the invention provides a library wherein the distribution of amino acids at the positions to be varied mimics that seen in the antigen binding site of antibodies. Such bias in the substitution of amino acids that permits selection of certain polypeptides (not just antibody polypeptides) against a range of target ligands is easily applied to any polypeptide repertoire according to the invention. There are various methods for biasing the amino acid distribution at the position to be varied (including the use of tri-nucleotide mutagenesis, WO97/08320, Morphosys, supra), of which the preferred method, due to ease of synthesis, is the use of conventional degenerate codons. By comparing the amino acid profile encoded by all combinations of degenerate codons (with single, double, triple and quadruple degeneracy in equal ratios at each position) with the natural amino acid use it is possible to calculate the most representative codon. The codons (AGT)(AGC)T, (AGT)(AGC)C and (AGT)(AGC)(CT)—that is, DVT, DVC and DVY, respectively using IUPAC nomenclature—are those closest to the desired amino acid profile: they encode 22% serine and 11% tyrosine, asparagine, glycine, alanine, aspartate, threonine and cysteine. Preferably, therefore, libraries are constructed using either the DVT, DVC or DVY codon at each of the diversified positions.

As stated above, polypeptides which make up antibody libraries according to the invention may be whole antibodies or fragments thereof, such as Fab, F(ab′)₂, Fv or scFv fragments, or separate VH or VL domains, any of which is either modified or unmodified. Of these, single-chain Fv fragments, or scFvs, are of particular use. ScFv fragments, as well as other antibody polypeptides, are reliably generated by antibody engineering methods well known in the art. The scFv is formed by connecting the VH and VL genes using an oligonucleotide that encodes an appropriately designed linker peptide, such as (Gly-Gly-Gly-Gly-Ser)₃ or equivalent linker peptide(s). The linker bridges the C-terminal end of the first V region and N-terminal end of the second V region, ordered as either VH-linker-VL or VL-linker-VH. In principle, the binding site of the scFv can faithfully reproduce the specificity of the corresponding whole antibody and vice-versa.

Similar techniques for the construction of Fv, Fab and F(ab′)₂ fragments, as well as chimeric antibody molecules are well known in the art. When expressing Fv fragments, precautions should be taken to ensure correct chain folding and association. For Fab and F(ab′)₂ fragments, VH and VL polypeptides are combined with constant region segments, which may be isolated from rearranged genes, germline C genes or synthesised from antibody sequence data as for V region segments. A library according to the invention may be a VH or VL library. Thus, separate libraries comprising single VH and VL domains may be constructed and, optionally, include C_{H} or C_{L} domains, respectively, creating Dab molecules.

c. Library Vector Systems According to the Invention

Libraries according to the invention can be used for direct screening using the generic and/or target ligands or used in a selection protocol that involves a genetic display package.

Bacteriophage lambda expression systems may be screened directly as bacteriophage plaques or as colonies of lysogens, both as previously described (Huse et al. (1989) Science, 246: 1275; Caton and Koprowski (1990) Proc. Natl. Acad. Sci. U.S.A., 87; Mullinax et al. (1990) Proc. Natl. Acad. Sci. U.S.A., 87: 8095; Persson et al. (1991) Proc. Natl. Acad. Sci. U.S.A., 88: 2432) and are of use in the invention. Whilst such expression systems can be used to screening up to 10⁶ different members of a library, they are not really suited to screening of larger numbers (greater than 10⁶ members). Other screening systems rely, for example, on direct chemical synthesis of library members. One early method involves the synthesis of peptides on a set of pins or rods, such as described in WO84/03564. A similar method involving peptide synthesis on beads, which forms a peptide library in which each bead is an individual library member, is described in U.S. Pat. No. 4,631,211 and a related method is described in WO92/00091. A significant improvement of the bead-based methods involves tagging each bead with a unique identifier tag, such as an oligonucleotide, so as to facilitate identification of the amino acid sequence of each library member. These improved bead-based methods are described in WO93/06121.

Another chemical synthesis method involves the synthesis of arrays of peptides (or peptidomimetics) on a surface in a manner that places each distinct library member (e.g., unique peptide sequence) at a discrete, predefined location in the array. The identity of each library member is determined by its spatial location in the array. The locations in the array where binding interactions between a predetermined molecule (e.g., a receptor) and reactive library members occur is determined, thereby identifying the sequences of the reactive library members on the basis of spatial location. These methods are described in U.S. Pat. No. 5,143,854; WO90/15070 and WO92/10092; Fodor et al. (1991) Science, 251: 767; Dower and Fodor (1991) Ann. Rep. Med. Chem., 26: 271.

Of particular use in the construction of libraries of the invention are selection display systems, which enable a nucleic acid to be linked to the polypeptide it expresses. As used herein, a selection display system is a system that permits the selection, by suitable display means, of the individual members of the library by binding the generic and/or target ligands.

Any selection display system may be used in conjunction with a library according to the invention. Selection protocols for isolating desired members of large libraries are known in the art, as typified by phage display techniques. Such systems, in which diverse peptide sequences are displayed on the surface of filamentous bacteriophage (Scott and Smith (1990) supra), have proven useful for creating libraries of antibody fragments (and the nucleotide sequences that encoding them) for the in vitro selection and amplification of specific antibody fragments that bind a target antigen. The nucleotide sequences encoding the VH and VL regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage-based display systems is that, because they are biological systems, selected library members can be amplified simply by growing the phage containing the selected library member in bacterial cells. Furthermore, since the nucleotide sequence that encode the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al. (1990) supra; Kang et al. (1991) Proc. Natl. Acad. Sci. U.S.A., 88: 4363; Clackson et al. (1991) Nature, 352: 624; Lowman et al. (1991) Biochemistry, 30: 10832; Burton et al. (1991) Proc. Natl. Acad. Sci U.S.A., 88: 10134; Hoogenboom et al. (1991) Nucleic Acids Res., 19: 4133; Chang et al. (1991) J. Immunol., 147: 3610; Breitling et al. (1991) Gene, 104: 147; Marks et al. (1991) supra; Barbas et al. (1992) supra; Hawkins and Winter (1992) J. Immunol., 22: 867; Marks et al., 1992, J. Biol. Chem., 267: 16007; Lerner et al. (1992) Science, 258: 1313, incorporated herein by reference).

One particularly advantageous approach has been the use of scFv phage-libraries (Huston et al., 1988, Proc. Natl. Acad. Sci. U.S.A., 85: 5879-5883; Chaudhary et al. (1990) Proc. Natl. Acad. Sci U.S.A., 87: 1066-1070; McCafferty et al. (1990) supra; Clackson et al. (1991) supra; Marks et al. (1991) supra; Chiswell et al. (1992) Trends Biotech., 10: 80; Marks et al. (1992) supra). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys, supra), which are incorporated herein by reference.

Other systems for generating libraries of polypeptides or nucleotides involve the use of cell-free enzymatic machinery for the in vitro synthesis of the library members. In one method, RNA molecules are selected by alternate rounds of selection against a target ligand and PCR amplification (Tuerk and Gold (1990) Science, 249: 505; Ellington and Szostak (1990) Nature, 346: 818). A similar technique may be used to identify DNA sequences which bind a predetermined human transcription factor (Thiesen and Bach (1990) Nucleic Acids Res., 18: 3203; Beaudry and Joyce (1992) Science, 257: 635; WO92/05258 and WO92/14843). In a similar way, in vitro translation can be used to synthesise polypeptides as a method far generating large libraries. These methods which generally comprise stabilised polysome complexes, are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax) use the polysomes to display polypeptides for selection. These and all the foregoing documents also are incorporated herein by reference.

The invention accordingly provides a method for selecting a polypeptide having a desired generic and/or target ligand binding site from a repertoire of polypeptides, comprising the steps of:
a) expressing a library according to the preceding aspects of the invention;
b) contacting the polypeptides with the generic and/or target ligand and selecting those which bind the generic and/or target ligand; and
c) optionally amplifying the selected polypeptide(s) which bind the generic and/or target ligand.
d) optionally repeating steps a)-c).

Preferably, steps a)-d) are performed using a phage display system.

Since the invention provides a library of polypeptides which have binding sites for both generic and target ligands the above selection method can be applied to a selection using either the generic ligand or the target ligand. Thus, the initial library can be selected using the generic ligand and then the target ligand or using the target ligand and then the generic ligand. The invention also provides for multiple selections using different generic ligands either in parallel or in series before or after selection with the target ligand.

Preferably, the method according to the invention further comprises the steps of subjecting the selected polypeptide(s) to additional variation (as described herein) and repeating steps a) to d).

Since the generic ligand, by its very nature, is able to bind all library members selected using the generic ligand, the method according to the invention further comprises the use of the generic ligand (or some conjugate thereof) to detect, immobilise, purify or immunoprecipitate any functional member or population of members from the library (whether selected by binding the target ligand or not).

Since the invention provides a library in which the members have a known main-chain conformation the method according to the invention further comprises the production of a three-dimensional structural model of any functional member of the library (whether selected by binding the target ligand or not). Preferably, the building of such a model involves homology modelling and/or molecular replacement. A preliminary model of the main-chain conformation can be created by comparison of the polypeptide sequence to the sequence of a known three-dimensional structure, by secondary structure prediction or by screening structural libraries. Computational software may also be used to predict the secondary structure of the polypeptide. In order to predict the conformations of the side-chains at the varied positions, a side-chain rotamer library may be employed.

In general, the nucleic acid molecules and vector constructs required for the performance of the present invention are available in the art and may be constructed and manipulated as set forth in standard laboratory manuals, such as Sambrook et al. (1989) Molecular Cloning: A Laboratory Manual, Cold Spring Harbor, USA.

The manipulation of nucleic acids in the present invention is typically carried out in recombinant vectors. As used herein, vector refers to a discrete element that is used to introduce heterologous DNA into cells for the expression and/or replication thereof. Methods by which to select or construct and, subsequently, use such vectors are well known to one of moderate skill in the art. Numerous vectors are publicly available, including bacterial plasmids, bacteriophage, artificial chromosomes and episomal vectors. Such vectors may be used for simple cloning and mutagenesis; alternatively, as is typical of vectors in which repertoire (or pre-repertoire) members of the invention are carried, a gene expression vector is employed. A vector of use according to the invention may be selected to accommodate a polypeptide coding sequence of a desired size, typically from 0.25 kilobase (kb) to 40 kb in length. A suitable host cell is transformed with the vector after in vitro cloning manipulations. Each vector contains various functional components, which generally include a cloning (or “polylinker”) site, an origin of replication and at least one selectable marker gene. If given vector is an expression vector, it additionally possesses one or more of the following: enhancer element, promoter, transcription termination and signal sequences, each positioned in the vicinity of the cloning site, such that they are operatively linked to the gene encoding a polypeptide repertoire member according to the invention.

Both cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g. SV 40, adenovirus) are useful for cloning vectors in mammalian cells. Generally, the origin of replication is not needed for mammalian expression vectors unless these are used in mammalian cells able to replicate high levels of DNA, such as COS cells.

Advantageously, a cloning or expression vector may contain a selection gene also referred to as selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

Since the replication of vectors according to the present invention is most conveniently performed in E. coli, an E. coli-selectable marker, for example, the β-lactamase gene that confers resistance to the antibiotic ampicillin, is of use. These can be obtained from E. coli plasmids, such as pBR322 or a pUC plasmid such as pUC18 or pUC19.

Expression vectors usually contain a promoter that is recognised by the host organism and is operably linked to the coding sequence of interest. Such a promoter may be inducible or constitutive. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the □-lactamase and lactose promoter systems, alkaline phosphatase, the tryptophan (trp) promoter system and hybrid promoters such as the tac promoter. Promoters for use in bacterial systems will also generally contain a Shine-Dalgarno sequence operably linked to the coding sequence.

In the library according to the present invention, the preferred vectors are expression vectors that enables the expression of a nucleotide sequence corresponding to a polypeptide library member. Thus, selection with the generic and/or target ligands can be performed by separate propagation and expression of a single clone expressing the polypeptide library member or by use of any selection display system. As described above, the preferred selection display system is bacteriophage display. Thus, phage or phagemid vectors may be used. The preferred vectors are phagemid vectors which have an E. coli. origin of replication (for double stranded replication) and also a phage origin of replication (for production of single-stranded DNA). The manipulation and expression of such vectors is well known in the art (Hoogenboom and Winter (1992) supra; Nissim et al. (1994) supra). Briefly, the vector contains a β-lactamase gene to confer selectivity on the phagemid and a lac promoter upstream of a expression cassette that consists (N to C terminal) of a pelB leader sequence (which directs the expressed polypeptide to the periplasmic space), a multiple cloning site (for cloning the nucleotide version of the library member), optionally, one or more peptide tag (for detection), optionally, one or more TAG stop codon and the phage protein pIII. Thus, using various suppressor and non-suppressor strains of E. coli and with the addition of glucose, isopropyl thio-β-D-galactoside (IPTG) or a helper phage, such as VCS M13, the vector is able to replicate as a plasmid with no expression, produce large quantities of the polypeptide library member only or produce phage, some of which contain at least one copy of the polypeptide-pIII fusion on their surface.

Construction of vectors according to the invention employs conventional ligation techniques. Isolated vectors or DNA fragments are cleaved, tailored, and religated in the form desired to generate the required vector. If desired, analysis to confirm that the correct sequences are present in the constructed vector can be performed in a known fashion. Suitable methods for constructing expression vectors, preparing in vitro transcripts, introducing DNA into host cells, and performing analyses for assessing expression and function are known to those skilled in the art. The presence of a gene sequence in a sample is detected, or its amplification and/or expression quantified by conventional methods, such as Southern or Northern analysis, Western blotting, dot blotting of DNA, RNA or protein, in situ hybridization, immunocytochemistry or sequence analysis of nucleic acid or protein molecules. Those skilled in the art will readily envisage how these methods may be modified, if desired.

### Mutagenesis Using the Polymerase Chain Reaction (PCR)

Once a vector system is chosen and one or more nucleic acid sequences encoding polypeptides of interest are cloned into the library vector, one may generate diversity within the cloned molecules by undertaking mutagenesis prior to expression; alternatively, the encoded proteins may be expressed and selected, as described above, before mutagenesis and additional rounds of selection are performed. As stated above, mutagenesis of nucleic acid sequences encoding structurally optimized polypeptides, is carried out by standard molecular methods. Of particular use is the polymerase chain reaction, or PCR, (Mullis and Faloona (1987) Methods Enzymol., 155: 335, herein incorporated by reference). PCR, which uses multiple cycles of DNA replication catalyzed by a thermostable, DNA-dependent DNA polymerase to amplify the target sequence of interest, is well known in the art.

Oligonucleotide primers useful according to the invention are single-stranded DNA or RNA molecules that hybridize to a nucleic acid template to prime enzymatic synthesis of a second nucleic acid strand. The primer is complementary to a portion of a target molecule present in a pool of nucleic acid molecules used in the preparation of sets of arrays of the invention. It is contemplated that such a molecule is prepared by synthetic methods, either chemical or enzymatic. Alternatively, such a molecule or a fragment thereof is naturally occurring, and is isolated from its natural source or purchased from a commercial supplier. Mutagenic oligonucleotide primers are 15 to 100 nucleotides in length, ideally from 20 to 40 nucleotides, although oligonucleotides of different length are of use.

Typically, selective hybridization occurs when two nucleic acid sequences are substantially complementary (at least about 65% complementary over a stretch of at least 14 to 25 nucleotides, preferably at least about 75%, more preferably at least about 90% complementary). See Kanehisa (1984) Nucleic Acids Res. 12: 203, incorporated herein by reference. As a result, it is expected that a certain degree of mismatch at the priming site is tolerated. Such mismatch may be small, such as a mono-, di- or tri-nucleotide. Alternatively, it may comprise nucleotide loops, which we define as regions in which mismatch encompasses an uninterrupted series of four or more nucleotides.

Overall, five factors influence the efficiency and selectivity of hybridization of the primer to a second nucleic acid molecule. These factors, which are (i) primer length, (ii) the nucleotide sequence and/or composition, (iii) hybridization temperature, (iv) buffer chemistry and (v) the potential for steric hindrance in the region to which the primer is required to hybridize, are important considerations when non-random priming sequences are designed.

There is a positive correlation between primer length and both the efficiency and accuracy with which a primer will anneal to a target sequence; longer sequences have a higher melting temperature (T_{M}) than do shorter ones, and are less likely to be repeated within a given target sequence, thereby minimizing promiscuous hybridization. Primer sequences with a high G-C content or that comprise palindromic sequences tend to self-hybridize, as do their intended target sites, since unimolecular, rather than bimolecular, hybridization kinetics are generally favoured in solution; at the same time, it is important to design a primer containing sufficient numbers of G-C nucleotide pairings to bind the target sequence tightly, since each such pair is bound by three hydrogen bonds, rather than the two that are found when A and T bases pair. Hybridization temperature varies inversely with primer annealing efficiency, as does the concentration of organic solvents, e.g. formamide that might be included in a hybridization mixture, while increases in salt concentration facilitate binding. Under stringent hybridization conditions, longer probes hybridize more efficiently than do shorter ones, which are sufficient under more permissive conditions. Stringent hybridization conditions typically include salt concentrations of less than about 1M, more usually less than about 500 mM and preferably less than about 200 mM. Hybridization temperatures range from as low as 0° C. to greater than 22° C., greater than about 30° C., and (most often) in excess of about 37° C. Longer fragments may require higher hybridization temperatures for specific hybridization. As several factors affect the stringency of hybridization, the combination of parameters is more important than the absolute measure of any one alone.

Primers are designed with these considerations in mind. While estimates of the relative merits of numerous sequences may be made mentally by one of skill in the art, computer programs have been designed to assist in the evaluation of these several parameters and the optimization of primer sequences. Examples of such programs are “PrimerSelect” of the DNAStar™ software package (DNAStar, Inc.; Madison, Wis.) and OLIGO 4.0 (National Biosciences, Inc.). Once designed, suitable oligonucleotides are prepared by a suitable method, e.g. the phosphoramidite method described by Beaucage and Carruthers (1981) Tetrahedron Lett., 22: 1859) or the triester method according to Matteucci and Caruthers (1981) J. Am. Chem. Soc., 103: 3185, both incorporated herein by reference, or by other chemical methods using either a commercial automated oligonucleotide synthesizer or VLSIPS™ technology.

PCR is performed using template DNA (at least 1 fg; more usefully, 1-1000 ng) and at least 25 pmol of oligonucleotide primers; it may be advantageous to use a larger amount of primer when the primer pool is heavily heterogeneous, as each sequence is represented by only a small fraction of the molecules of the pool, and amounts become limiting in the later amplification cycles. A typical reaction mixture includes: 2 μl of DNA, 25 pmol of oligonucleotide primer, 2.5 μl of 10×PCR buffer 1 (Perkin-Elmer, Foster City, Calif.), 0.4 μl of 1.25 μM dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler.

The length and temperature of each step of a PCR cycle, as well as the number of cycles, is adjusted in accordance to the stringency requirements in effect. Annealing temperature and timing are determined both by the efficiency with which a primer is expected to anneal to a template and the degree of mismatch that is to be tolerated; obviously, when nucleic acid molecules are simultaneously amplified and mutagenized, mismatch is required, at least in the first round of synthesis. In attempting to amplify a population of molecules using a mixed pool of mutagenic primers, the loss, under stringent (high-temperature) annealing conditions, of potential mutant products that would only result from low melting temperatures is weighed against the promiscuous annealing of primers to sequences other than the target site. The ability to optimize the stringency of primer annealing conditions is well within the knowledge of one of moderate skill in the art. An annealing temperature of between 30 C and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1-5 minutes, depending on the length of the amplified product). Final extension is generally for 4 minutes at 72° C., and may be followed by an indefinite (0-24 hour) step at 4° C.

### Structural Analysis of Repertoire Members

Since the invention provides a repertoire of polypeptides of known main-chain conformation, a three-dimensional structural model of any member of the repertoire is easily generated. Typically, the building of such a model involves homology modelling and/or molecular replacement. A preliminary model of the main-chain conformation is created by comparison of the polypeptide sequence to a similar sequence of known three-dimensional structure, by secondary structure prediction or by screening structural libraries. Molecular modelling computer software packages are commercially available, and are useful in predicting polypeptide secondary structures. In order to predict the conformations of the side-chains at the varied positions, a side-chain rotamer library may be employed.

### Antibodies for Use as Ligands in Polypeptide Selection

A generic or target ligand to be used in the polypeptide selection according to the present invention may, itself, be an antibody. This is particularly true of generic ligands, which bind to structural features that are substantially conserved in functional polypeptides to be selected for inclusion in repertoires of the invention. If an appropriate antibody is not publicly available, it may be produced by phage display methodology (see above) or as follows:

Either recombinant proteins or those derived from natural sources can be used to generate antibodies using standard techniques, well known to those in the field. For example, the protein (or “immunogen”) is administered to challenge a mammal such as a monkey, goat, rabbit or mouse. The resulting antibodies can be collected as polyclonal sera, or antibody-producing cells from the challenged animal can be immortalized (e.g. by fusion with an immortalizing fusion partner to produce a hybridoma), which cells then produce monoclonal antibodies.

a. Polyclonal Antibodies

The antigen protein is either used alone or conjugated to a conventional carrier in order to increases its immunogenicity, and an antiserum to the peptide-carrier conjugate is raised in an animal, as described above. Coupling of a peptide to a carrier protein and immunizations may be performed as described (Dymecki et al. (1992) J. Biol. Chem., 267: 4815). The serum is titered against protein antigen by ELISA or alternatively by dot or spot blotting (Boersma and Van Leeuwen (1994) J. Neurosci. Methods, 51: 317). The serum is shown to react strongly with the appropriate peptides by ELISA, for example, following the procedures of Green et al. (1982) Cell, 28: 477.

b. Monoclonal Antibodies

Techniques for preparing monoclonal antibodies are well known, and monoclonal antibodies may be prepared using any candidate antigen, preferably bound to a carrier, as described by Arnheiter et al. (1981) Nature, 294, 278. Monoclonal antibodies are typically obtained from hybridoma tissue cultures or from ascites fluid obtained from animals into which the hybridoma tissue was introduced. Nevertheless, monoclonal antibodies may be described as being “raised against” or “induced by” a protein.

After being raised, monoclonal antibodies are tested for function and specificity by any of a number of means. Similar procedures can also be used to test recombinant antibodies produced by phage display or other in vitro selection technologies. Monoclonal antibody-producing hybridomas (or polyclonal sera) can be screened for antibody binding to the immunogen, as well. Particularly preferred immunological tests include enzyme-linked immunoassays (ELISA), immunoblotting and immunoprecipitation (see Voller, (1978) Diagnostic Horizons, 2: 1, Microbiological Associates Quarterly Publication, Walkersville, Md.; Voller et al. (1978) J. Clin. Pathol., 31: 507; U.S. Reissue Pat. No. 31,006; UK Patent 2,019,408; Butler (1981) Methods Enzymol., 73: 482; Maggio, E. (ed.), (1980) Enzyme Immunoassay, CRC Press, Boca Raton, Fla.) or radioimmunoassays (RIA) (Weintraub, B., Principles of radioimmunoassays, Seventh Training Course on Radioligand Assay Techniques, The Endocrine Society, March 1986, pp. 1-5, 46-49 and 68-78), all to detect binding of the antibody to the immunogen against which it was raised. It will be apparent to one skilled in the art that either the antibody molecule or the immunogen must be labelled to facilitate such detection. Techniques for labelling antibody molecules are well known to those skilled in the art (see Harlour and Lane (1989) Antibodies, Cold Spring Harbor Laboratory, pp. 1-726).

Alternatively, other techniques can be used to detect binding to the immunogen, thereby confirming the integrity of the antibody which is to serve either as a generic antigen or a target antigen according to the invention. These include chromatographic methods such as SDS PAGE, isoelectric focusing, Western blotting, HPLC and capillary electrophoresis.

“Antibodies” are defined herein as constructions using the binding (variable) region of such antibodies, and other antibody modifications. Thus, an antibody useful in the invention may comprise whole antibodies, antibody fragments, polyfunctional antibody aggregates, or in general any substance comprising one or more specific binding sites from an antibody. The antibody fragments may be fragments such as Fv, Fab and F(ab′)₂ fragments or any derivatives thereof, such as a single chain Fv fragments. The antibodies or antibody fragments may be non-recombinant, recombinant or humanized. The antibody may be of any immunoglobulin isotype, e.g., IgG, IgM, and so forth. In addition, aggregates, polymers, derivatives and conjugates of immunoglobulins or their fragments can be used where appropriate.

The invention is further described, for the purposes of illustration only, in the following examples.

### Metallic Ions as Ligands for the Selection of Polypeptides

As stated above, ligands other than antibodies are of use in the selection of polypeptides according to the invention. One such category of ligand is that of metallic ions. For example, one may wish to preselect a repertoire for the presence of a functional histidine (HIS) tag using a Ni-NTA matrix. Immobilized metal affinity chromatography (IMAC; Hubert and Porath (1980) J. Chromatography, 98: 247) takes advantage of the metal-binding properties of histidine and cysteine amino acid residues, as well as others that may bind metals, on the exposed surfaces of numerous proteins. It employs a resin, typically agarose, comprising a bidentate metal chelator (e.g. iminodiacetic acid, IDA, a dicarboxylic acid group) to which is complexed metallic ions; in order to generate a metallic-ion-bearing resin according to the invention, agarose/IDA is mixed with a metal salt (for example, CuCl₂2H₂O), from which the IDA chelates the divalent cations. One commercially available agarose/IDA preparation is “CHELATING SEPHAROSE 6B” (Pharmacia Fine Chemicals; Piscataway, N.J.). Metallic ion that are of use include, but are not limited to, the divalent cations Ni²⁺, Cu²⁺, Zn²⁺ and Co²⁺. A pool of polypeptide molecules is prepared in a binding buffer which consists essentially of salt (typically, NaCl or KCl) at a 0.1- to 1.0M concentration and a weak ligand (such as Tris or ammonia), the latter of which has affinity for the metallic ions of the resin, but to a lesser degree than does a polypeptide to be selected according to the invention. Useful concentrations of the weak ligand range from 0.01- to 0.1M in the binding buffer.

The polypeptide pool is contacted with the resin under conditions which permit polypeptides having metal-binding domains (see below) to bind; after impurities are washed away, the selected polypeptides are eluted with a buffer in which the weak ligand is present in a higher concentration than in the binding buffer, specifically, at a concentration sufficient for the weak ligand to displace the selected polypeptides, despite its lower binding affinity for the metallic ions. Useful concentrations of the weak ligand in the elution buffer are 10- to 50-fold higher than in the binding buffer, typically from 0.1 to 0.3 M; note that the concentration of salt in the elution buffer equals that in the binding buffer. According to the methods of the present invention, the metallic ions of the resin typically serve as the generic ligand; however, it is contemplated that they may also be used as the target ligand.

IMAC is carried out using a standard chromatography apparatus (columns, through which buffer is drawn by gravity, pulled by a vacuum or driven by pressure); alternatively, a large-batch procedure is employed, in which the metal-bearing resin is mixed, in slurry form, with the polypeptide pool from which members of a repertoire of the invention are to be selected.

Partial purification of a serum T4 protein by IMAC has been described (Staples et al., U.S. Pat. No. 5,169,936); however, the broad spectrum of proteins comprising surface-exposed metal-binding domains also encompasses other soluble T4 proteins, human serum proteins (e.g. IgG, haptoglobulin, hemopexin, Gc-globulin, Clq, C3, C4), human desmoplasmin, Dolichos biflorus lectin, zinc-inhibited Tyr(P) phosphatases, phenolase, carboxypeptidase isoenzymes, Cu, Zn superoxide dismutases (including those of humans and all other eukaryotes), nucleoside diphosphatase, leukocyte interferon, lactoferrin, human plasma α₂-SH glycoprotein, β₂-macroglobulin, α₁-antitrypsin, plasminogen activator, gastrointestinal polypeptides, pepsin, human and bovine serum albumin, granule proteins from granulocytes, lysozymes, non-histone proteins, human fibrinogen, human serum transferrin, human lymphotoxin, calmodulin, protein A, avidin, myoglobulins, somatomedins, human growth hormone, transforming growth factors, platelet-derived growth factor, α-human atrial natriuretic polypeptide, cardiodilatin and others. In addition, extracellular domain sequences of membrane-bound proteins may be purified using IMAC. Note that repertoires comprising any of the above proteins or metal-binding variants thereof may be produced according to the methods of the invention.

Following elution, selected polypeptides are removed from the metal binding buffer and placed in a buffer appropriate to their next use. If the metallic ion has been used to generate a first selected polypeptide pool according to the invention, the molecules of that pool are placed into a buffer that is optimized for binding with the second ligand to be used in selection of the members of the functional polypeptide repertoire. If the metal is, instead, used in the second selection step, the polypeptides of the repertoire are transferred to a buffer suitable either to storage (e.g. a 0.5% glycine buffer) or the use for which they are intended. Such buffers include, but are not limited to: water, organic solvents, mixtures of water and water-miscible organic solvents, physiological salt buffers and protein/nucleic acid or protein/protein binding buffers. Alternatively, the polypeptide molecules may be dehydrated (i.e. by lyophilization) or immobilized on a solid or semi-solid support, such as a nitrocellulose or nylon filtration membrane or a gel matrix (i.e. of agarose or polyacrylamide) or crosslinked to a chromatography resin.

Polypeptide molecules may be removed from the elution buffer by any of a number of methods known in the art. The polypeptide eluate may be dialyzed against water or another solution of choice; if the polypeptides are to be lyophilized, water to which has been added protease inhibitors (e.g. pepstatin, aprotinin, leupeptin, or others) is used. Alternatively, the sample may be subjected to ammonium sulphate precipitation, which is well known in the art, prior to resuspension in the medium of choice.

### Use of Polypeptides Selected According to the Invention

Polypeptides selected according to the method of the present invention may be employed in substantially any process which involves ligand-polypeptide binding, including in vivo therapeutic and prophylactic applications, in vitro and in vivo diagnostic applications, in vitro assay and reagent applications, and the like. For example, in the case of antibodies, antibody molecules may be used in antibody based assay techniques, such as ELISA techniques, according to methods known to those skilled in the art.

As alluded to above, the molecules selected according to the invention are of use in diagnostic, prophylactic and therapeutic procedures. For example, enzyme variants generated and selected by these methods may be assayed for activity, either in vitro or in vivo using techniques well known in the art, by which they are incubated with candidate substrate molecules and the conversion of substrate to product is analyzed. Selected cell-surface receptors or adhesion molecules might be expressed in cultured cells which are then tested for their ability to respond to biochemical stimuli or for their affinity with other cell types that express cell-surface molecules to which the undiversified adhesion molecule would be expected to bind, respectively. Antibody polypeptides selected according to the invention are of use diagnostically in Western analysis and in situ protein detection by standard immunohistochemical procedures; for use in these applications, the antibodies of a selected repertoire may be labelled in accordance with techniques known to the art. In addition, such antibody polypeptides may be used preparatively in affinity chromatography procedures, when complexed to a chromatographic support, such as a resin. All such techniques are well known to one of skill in the art.

Therapeutic and prophylactic uses of proteins prepared according to the invention involve the administration of polypeptides selected according to the invention to a recipient mammal, such as a human. Of particular use in this regard are antibodies, other receptors (including, but not limited to T-cell receptors) and in the case in which an antibody or receptor was used as either a generic or target ligand, proteins which bind to them.

Substantially pure antibodies or binding proteins thereof of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Perris, (1979 and 1981) Immunological Methods, Volumes I and II, Academic Press, NY).

The selected antibodies or binding proteins thereof of the present invention will typically find use in preventing, suppressing or treating inflammatory states, allergic hypersensitivity, cancer, bacterial or viral infection, and autoimmune disorders (which include, but are not limited to, Type I diabetes, multiple sclerosis, rheumatoid arthritis, systemic lupus erythematosus, Crohn's disease and myasthenia gravis).

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the antibodies or binding proteins thereof in protecting against or treating the disease are available. Methods for the testing of systemic lupus erythematosus (SLE) in susceptible mice are known in the art (Knight et al. (1978) J. Exp. Med., 147: 1653; Reinersten et al. (1978) New Eng. J. Med., 299: 515). Myasthenia Gravis (MG) is tested in SJL/J female mice by inducing the disease with soluble AchR protein from another species (Lindstrom et al. (1988) Adv. Immunol., 42: 233). Arthritis is induced in a susceptible strain of mice by injection of Type II collagen (Stuart et al. (1984) Ann. Rev. Immunol., 42: 233). A model by which adjuvant arthritis is induced in susceptible rats by injection of mycobacterial heat shock protein has been described (Van Eden et al. (1988) Nature, 331: 171). Thyroiditis is induced in mice by administration of thyroglobulin as described (Maron et al. (1980) J. Exp. Med., 152: 1115). Insulin dependent diabetes mellitus (IDDM) occurs naturally or can be induced in certain strains of mice such as those described by Kanasawa et al. (1984) Diabetologia, 27: 113. EAE in mouse and rat serves as a model for MS in human. In this model, the demyelinating disease is induced by administration of myelin basic protein (see Paterson (1986) Textbook of Immunopathology, Mischer et al., eds., Grune and Stratton, New York, pp. 179-213; McFarlin et al. (1973) Science, 179: 478: and Satoh et al. (1987) J. Immunol., 138: 179).

The selected antibodies, receptors (including, but not limited to T-cell receptors) or binding proteins thereof of the present invention may also be used in combination with other antibodies, particularly monoclonal antibodies (MAbs) reactive with other markers on human cells responsible for the diseases. For example, suitable T-cell markers can include those grouped into the so-called “Clusters of Differentiation,” as named by the First International Leukocyte Differentiation Workshop (Bernhard et al. (1984) Leukocyte Typing, Springer Verlag, NY).

Generally, the present selected antibodies, receptors or binding proteins will be utilized in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The selected polypeptides of the present invention may be used as separately administered compositions or in conjunction with other agents. These can include various immunotherapeutic drugs, such as cyclosporine, methotrexate, adriamycin or cisplatin and immunotoxins. Pharmaceutical compositions can include “cocktails” of various cytotoxic or other agents in conjunction with the selected antibodies, receptors or binding proteins thereof of the present invention, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. For therapy, including without limitation immunotherapy, the selected antibodies, receptors or binding proteins thereof of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician.

The selected polypeptides of this invention can be lyophilized for storage and reconstituted in a suitable carrier prior to use. This technique has been shown to be effective with conventional immunoglobulins and art-known lyophilization and reconstitution techniques can be employed. It will be appreciated by those skilled in the art that lyophilization and reconstitution can lead to varying degrees of antibody activity loss (e.g. with conventional immunoglobulins, IgM antibodies tend to have greater activity loss than IgG antibodies) and that use levels may have to be adjusted upward to compensate.

The compositions containing the present selected polypeptides or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected antibody, receptor (e.g. a T-cell receptor) or binding protein thereof per kilogram of body weight, with doses of 0.05 to 2.0 mg/kg/dose being more commonly used. For prophylactic applications, compositions containing the present selected polypeptides or cocktails thereof may also be administered in similar or slightly lower dosages.

A composition containing a selected polypeptide according to the present invention may be utilized in prophylactic and therapeutic settings to aid in the alteration, inactivation, killing or removal of a select target cell population in a mammal. In addition, the selected repertoires of polypeptides described herein may be used extra corporeally or in vitro selectively to kill, deplete or otherwise effectively remove a target cell population from a heterogeneous collection of cells. Blood from a mammal may be combined extracorporeally with the selected antibodies, cell-surface receptors or binding proteins thereof whereby the undesired cells are killed or otherwise removed from the blood for return to the mammal in accordance with standard techniques.

The invention is further described, for the purposes of illustration only, in the following examples.

### Example 1

### Antibody Library Design

### A. Main-Chain Conformation

For five of the six antigen binding loops of human antibodies (L1, L2, L3, H1 and H2) there are a limited number of main-chain conformations, or canonical structures ((Chothia et al. (1992) J. Mol. Biol., 227: 799; Tomlinson et al. (1995) EMBO J., 14: 4628; Williams et al. (1996) J. Mol. Biol., 264: 220). The most popular main-chain conformation for each of these loops is used to provide a single known main-chain conformation according to the invention. These are: H1-CS 1 (79% of the expressed repertoire), H2-CS 3 (46%), L1-CS 2 of V_{κ} (39%), L2-CS 1 (100%), L3-CS 1 of V_{κ} (36%). The H3 loop forms a limited number of main-chain conformations for short loop lengths (Martin et al. (1996) J. Mol. Biol., 263: 800; Shirai et al. (1996) FEBS Letters, 399: 1). Thus, where the H3 has a CDR3 length (as defined by Kabat et al. (1991). Sequences of proteins of immunological interest, U.S. Department of Health and Human Services) of seven residues and has a lysine or arginine residue at position H94 and an aspartate residue at position H101 a salt-bridge is formed between these two residues and in most cases a single main-chain conformation is likely to be produced. There are at least 16 human antibody sequences in the EMBL data library with the required H3 length and key residues to form this conformation and at least two crystallographic structures in the protein data bank which can be used as a basis for antibody modelling (2cgr and 1tet).

In this case, the most frequently expressed germline gene segments which encode the desired loop lengths and key residues to produce the required combinations of canonical structures are the VH segment 3-23 (DP-47), the J_{H} segment JH4b, the V_{κ} segment O2/O12 (DPK9) and the J_{κ} segment J_{κ}1. These segments can therefore be used in combination as a basis to construct a library with the desired single main-chain conformation. The V_{κ} segment O2/O12 (DPK9) is member of the V_{κ}1 family and therefore will bind the superantigen Protein L. The VH segment 3-23 (DP-47) is a member of the VH3 family and therefore should bind the superantigen Protein A, which can then be used as a generic ligand.

### B. Selection of Positions for Variation

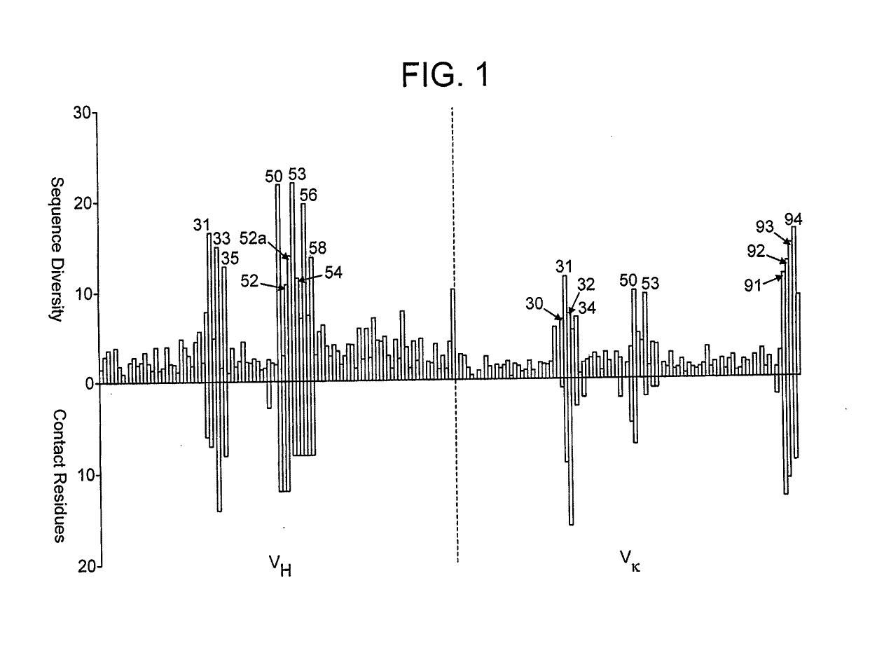

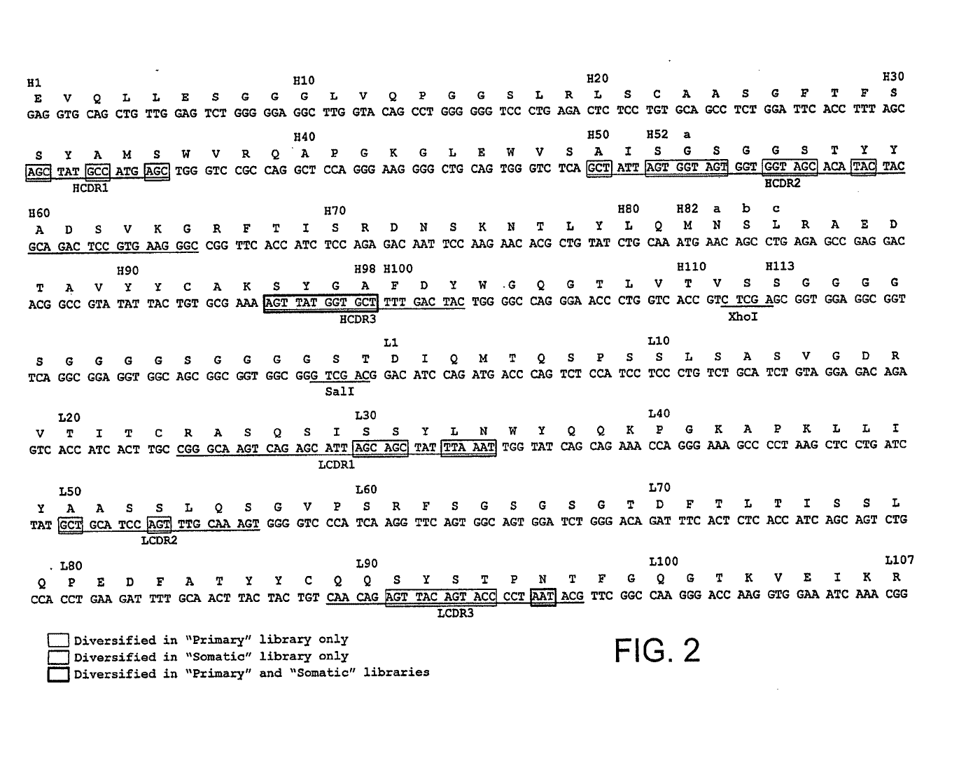

Analysis of human VH and V_{κ} sequences indicates that the most diverse positions in the mature repertoire are those that make the most contacts with antigens (see Tomlinson et al., (1996) J. Mol. Biol., 256: 813; FIG. 1). These positions form the functional antigen binding site and are therefore selected for side-chain diversification (FIG. 2). H54 is a key residue and points away from the antigen binding site in the chosen H2 canonical structure 3 (the diversity seen at this position is due to canonical structures 1, 2 and 4 where H54 points into the binding site). In this case H55 (which points into the binding site) is diversified instead. The diversity at these positions is created either by germline or junctional diversity in the primary repertoire or by somatic hypermutation (Tomlinson et al., (1996) J. Mol. Biol., 256: 813; FIG. 1). Two different subsets of residues in the antigen binding site were therefore varied to create two different library formats. In the “primary” library the residues selected for variation are from H2, H3, L2 and L3 (diversity in these loops is mainly the result of germline or junctional diversity). The positions varied in this library are: H50, H52, H52a, H53, H55, H56, H58, H95, H96, H97, H98, L50, L53, L91, L92, L93, L94 and L96 (18 residues in total, FIG. 2). In the “somatic” library the residues selected for variation are from H1, H3, L1 and the end of L3 (diversity here is mainly the result of somatic hypermutation or junctional diversity). The positions varied in this library are: H31, H33, H35, H95, H96, H97, H98, L30, L31, L32, L34 and L96 (12 residues in total, FIG. 2).

### C. Selection of Amino Acid Use at the Positions to be Varied

Side-chain diversity is introduced into the “primary” and “somatic” libraries by incorporating either the codon NNK (which encodes all 20 amino acids, including the TAG stop codon, but not the TGA and TAA stop codons) or the codon DVT (which encodes 22% serine and 11% tyrosine, asparagine, glycine, alanine, aspartate, threonine and cysteine and using single, double, triple and quadruple degeneracy in equal ratios at each position, most closely mimics the distribution of amino acid residues for in the antigen binding sites of natural human antibodies).

### Example 2

### Library Construction and Selection with the Generic Ligands

The “primary” and “somatic” libraries were assembled by PCR using the oligonucleotides listed in Table 1 and the germline V gene segments DPK9 (Cox et al. (1994) Eur. J. Immunol., 24: 827) and DP-47 (Tomlinson et al. (1992) J. Mol. Biol., 227: 7768). Briefly, first round of amplification was performed using pairs of 5′ (back) primers in conjunction with NNK or DVT 3′ (forward) primers together with the corresponding germline V gene segment as template (see Table 1). This produces eight separate DNA fragments for each of the NNK and DVT libraries. A second round of amplification was then performed using the 5′ (back) primers and the 3′ (forward) primers shown in Table 1 together with two of the purified fragments from the first round of amplification. This produces four separate fragments for each of the NNK and DVT libraries (a “primary” VH fragment, 5A; a “primary” V_{κ} fragment, 6A; a “somatic” VH fragment, 5B; and a “somatic” V_{κ} fragment, 6B).

Each of these fragments was cut and then ligated into pCLEANVH (for the VH fragments) or pCLEANVK (for the V_{κ} fragments) which contain dummy VH and V_{κ} domains, respectively in a version of pHEN1 which does not contain any TAG codons or peptide tags (Hoogenboom & Winter (1992) J. Mol. Biol., 227: 381). The ligations were then electroporated into the non-suppressor E. Coli. strain HB2151. Phage from each of these libraries was produced and separately selected using immunotubes coated with 10 μg/ml of the generic ligands Protein A and Protein L for the VH and V_{κ} libraries, respectively. DNA from E. Coli. infected with selected phage was then prepared and cut so that the dummy V_{κ} inserts were replaced by the corresponding V_{κ} libraries. Electroporation of these libraries results in the following insert library sizes: 9.21×10⁸ (“primary” NNK), 5.57×10⁸ (“primary” DVT), 1.00×10⁹ (“somatic” NNK) and 2.38×10⁸ (“somatic” DVT). As a control for pre-selection four additional libraries were created but without selection with the generic ligands Protein A and Protein L: insert library sizes for these libraries were 1.29×10⁹ (“primary” NNK), 2.40×10⁸ (“primary” DVT), 1.16×10⁹ (“somatic” NNK) and 2.17×10⁸ (“somatic” DVT).

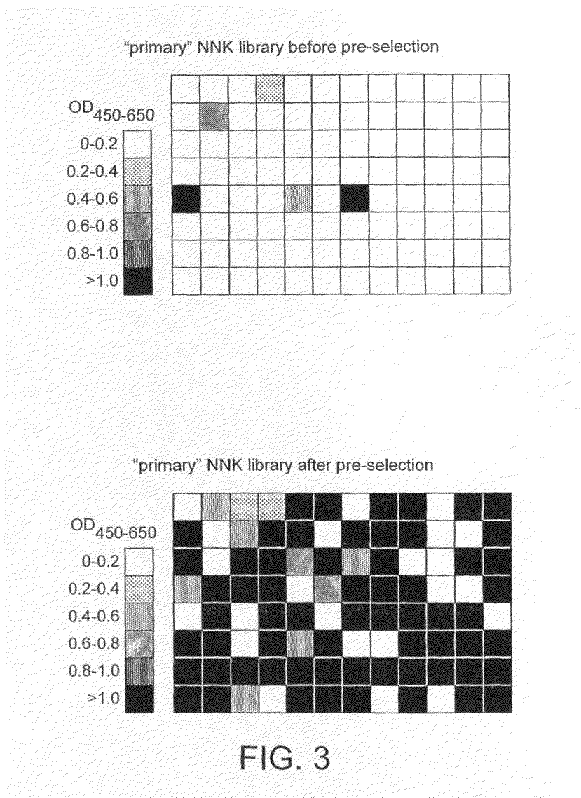

To verify the success of the pre-selection step, DNA from the selected and unselected “primary” NNK libraries was cloned into a pUC based expression vector and electroporated into HB2151. 96 clones were picked at random from each recloned library and induced for expression of soluble scFv fragments. Production of functional scFv is assayed by ELISA using Protein L to capture the scFv and then Protein A-HRP conjugate to detect binding. Only scFv which express functional VH and V_{κ} domains (no frame-shifts, stop codons, folding or expression mutations) will give a signal using this assay. The number of functional antibodies in each library (ELISA signals above background) was 5% with the unselected “primary” NNK library and 75% with the selected version of the same (FIG. 3). Sequencing of clones which were negative in the assay confirmed the presence of frame-shifts, stop codons, PCR mutations at critical framework residues and amino acids in the antigen binding site which must prevent folding and/or expression.

### Example 3

### Library Selection Against Target Ligands

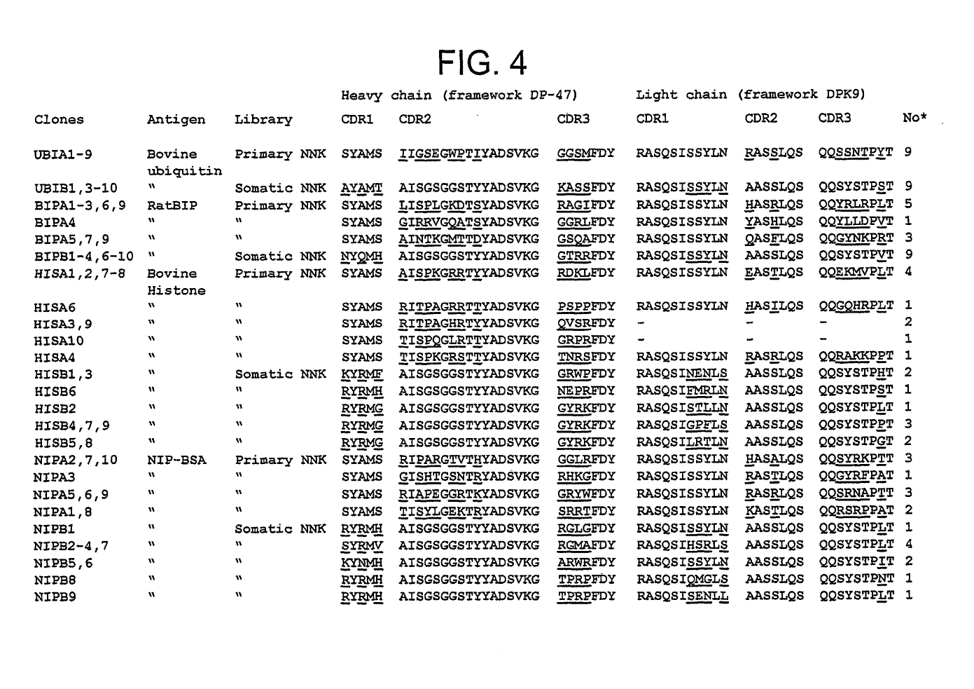

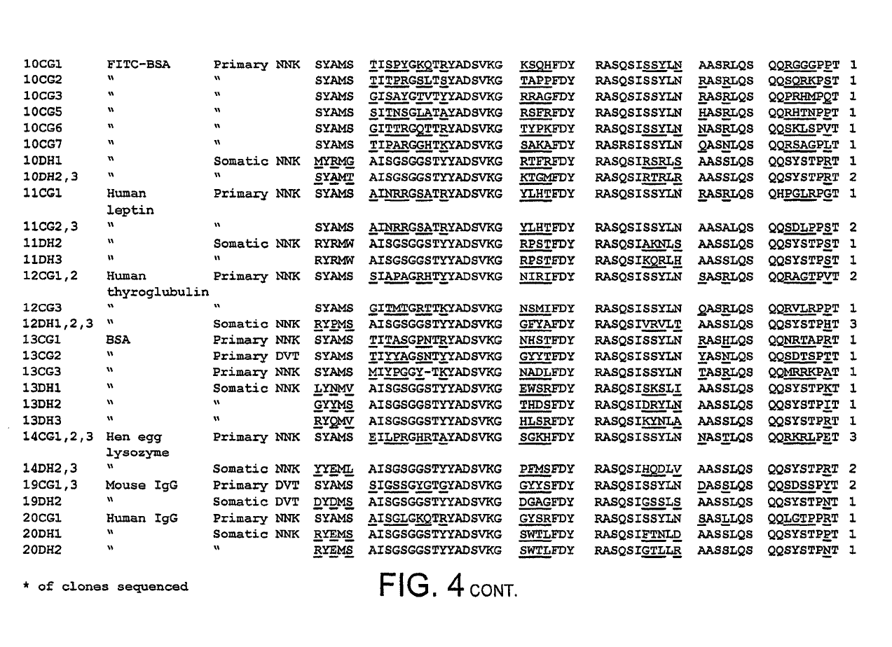

The “primary” and “somatic” NNK libraries (without pre-selection) were separately selected using five antigens (bovine ubiquitin, rat BIP, bovine histone, NIP-BSA and hen egg lysozyme) coated on immunotubes at various concentrations. After 2-4 rounds of selection, highly specific antibodies were obtained to all antigens except hen egg lysozyme. Clones were selected at random for sequencing demonstrating a range of antibodies to each antigen (FIG. 4).

In the second phase, phage from the pre-selected NNK and DVT libraries were mixed 1:1 to create a single “primary” library and a single “somatic” library. These libraries were then separately selected using seven antigens (FITC-BSA, human leptin, human thyroglobulin, BSA, hen egg lysozyme, mouse IgG and human IgG) coated on immunotubes at various concentrations. After 2-4 rounds of selection, highly specific antibodies were obtained to all the antigens, including hen egg lysozyme which failed to produce positives in the previous phase of selection using the libraries that had not been pre-selected using the generic ligands. Clones were selected at random for sequencing, demonstrating a range of different antibodies to each antigen (FIG. 4).

### Example 4

### Effect of Pre-Selection on scFv Expression and Production of Phage Bearing scFv

To further verify the outcome of the pre-selection, DNA from the unselected and pre-selected “primary” DVT libraries is cloned into a pUC based expression vector and electroporated into HB2151, yielding 10⁵ clones in both cases. 96 clones are picked at random from each recloned library and induced for expression of soluble scFv fragments. Production of functional scFv is again assayed using Protein L to capture the scFv followed by the use of Protein A-HRP to detect bound scFv. The percentage of functional antibodies in each library is 35.4% (unselected) and 84.4% (pre-selected) indicating a 2.4 fold increase in the number of functional members as a result of pre-selection with Protein A and Protein L (the increase is less pronounced than with the equivalent NNK library since the DVT codon does not encode the TAG stop codon. In the unselected NNK library, the presence of a TAG stop codon in a non-suppressor strain such as HB2151 will lead to termination and hence prevent functional scFv expression. Pre-selection of the NNK library removes clones containing TAG stop codons to produce a library in which a high proportion of members express soluble scFv.)

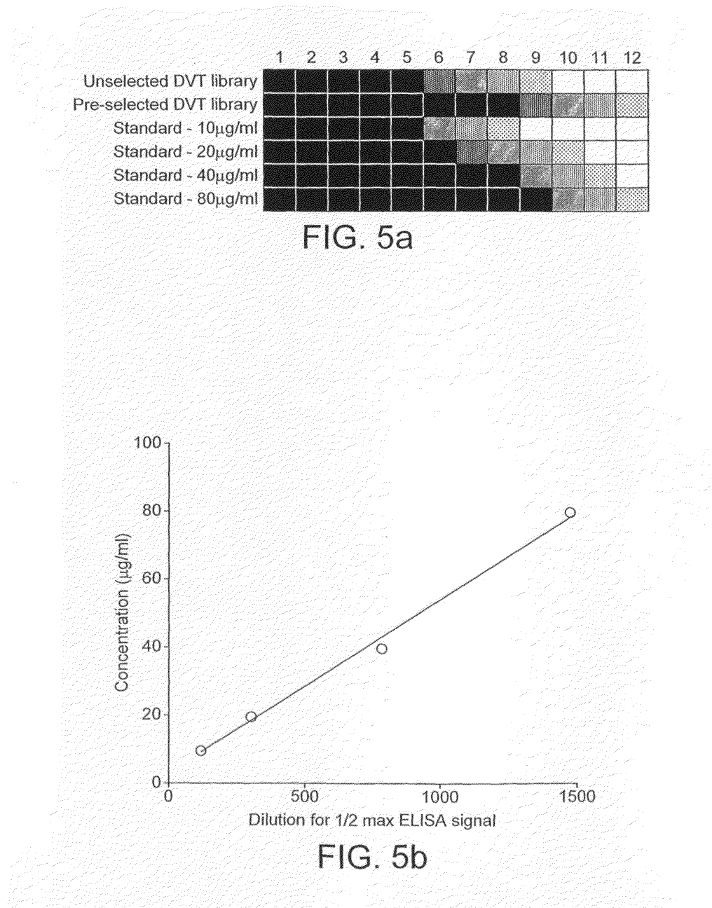

In order to assess the effect to pre-selection of the “primary” DVT library on total scFv expression, the recloned unselected and pre-selected libraries (each containing 10⁵ clones in a pUC based expression vector) are induced for polyclonal expression of scFv fragments. The concentration of expressed scFv in the supernatant is then determined by incubating two fold dilutions (columns 1-12 in FIG. 5a) of the supernatants on Protein L coated ELISA plate, followed by detection with Protein A-HRP, ScFvs of known concentration are assayed in parallel to quantify the levels of scFv expression in the unselected and pre-selected DVT libraries. These are used to plot a standard curve (FIG. 5b) and from this the expression levels of the unselected and pre-selected “primary” DVT libraries are calculated as 12.9 μg/ml and 67.1 μg/ml respectively i.e. a 5.2 fold increase in expression due to pre-selection with Protein A and Protein L.

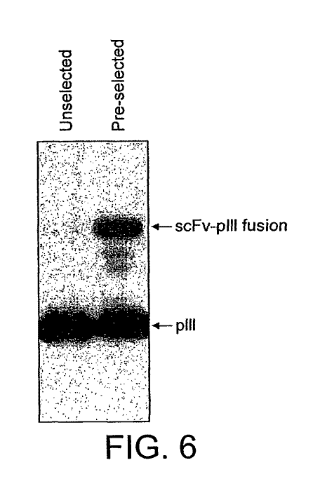

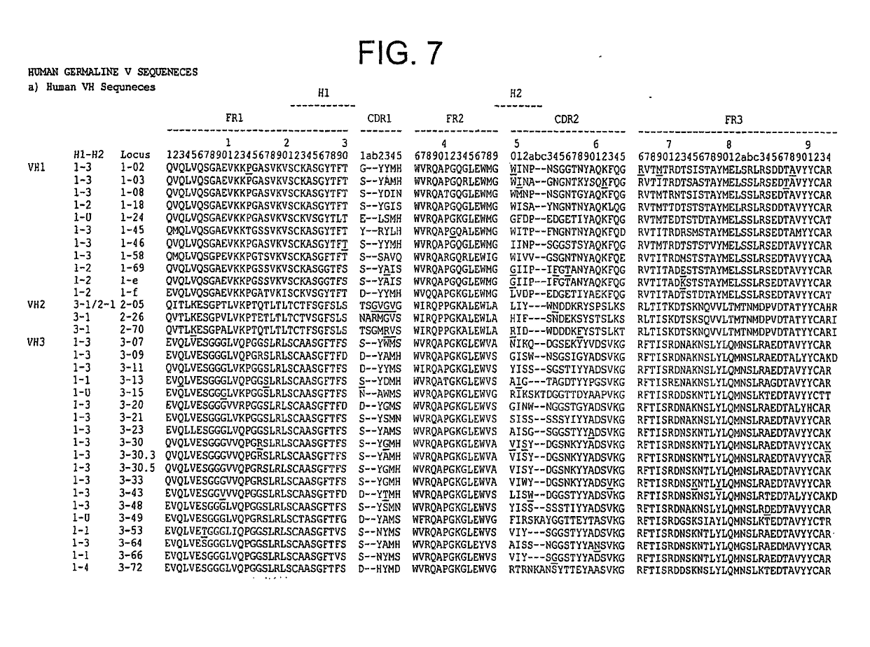

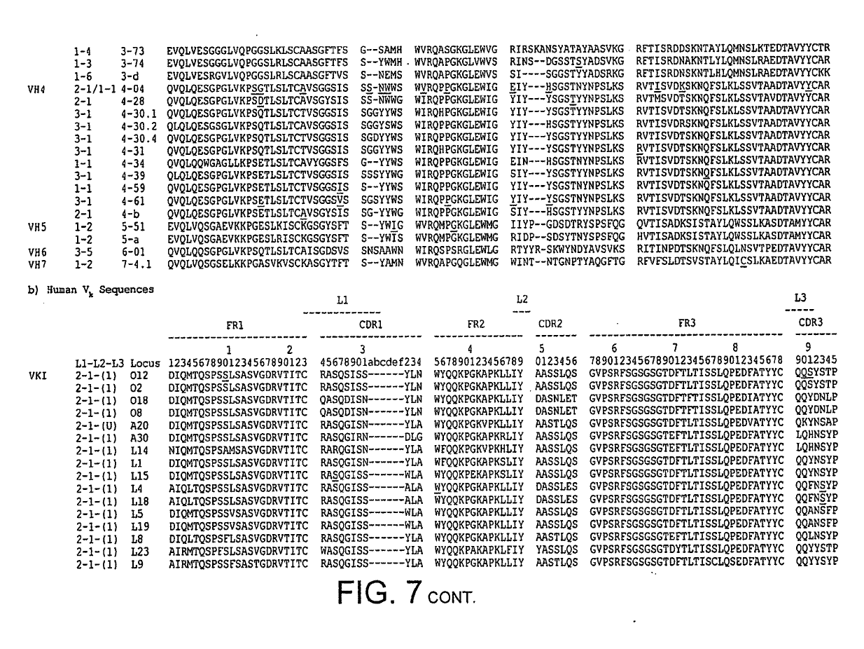

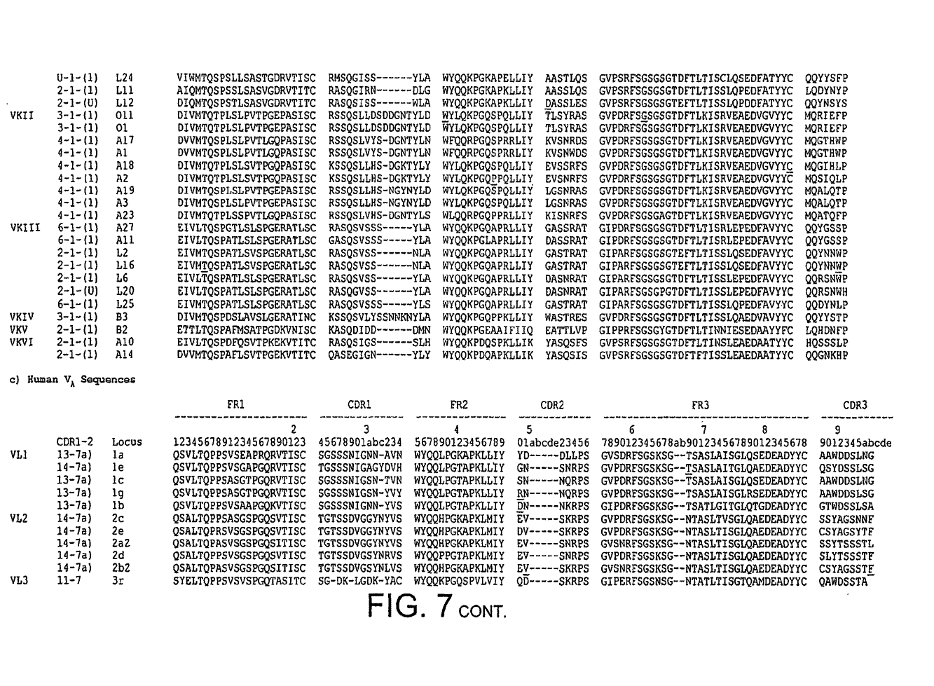

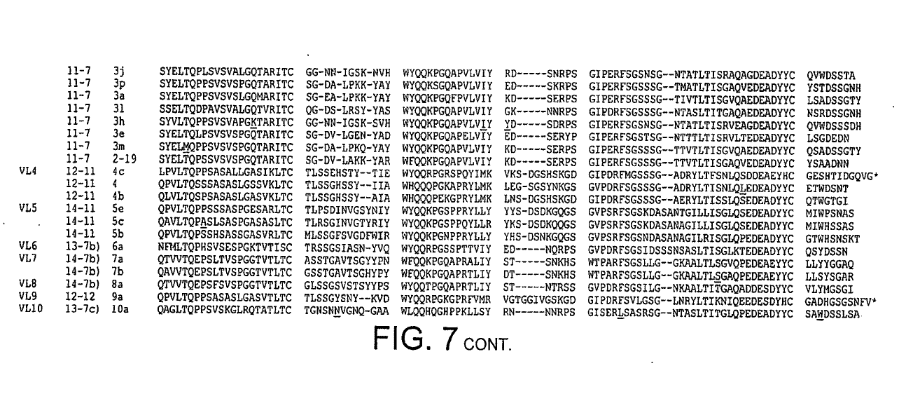

To assess the amount of phage bearing scFv, the unselected and pre-selected “primary” DVT libraries are grown and polyclonal phage is produced. Equal volumes of phage from the two libraries are run under denaturing conditions on a 4-12% Bis-Tris NuPAGE Gel with MES running buffer. The resulting gel is western blotted, probed using an anti-pIII antibody and exposed to X-ray film (FIG. 6). The lower band in each case corresponds to pIII protein alone, whilst the higher band contains the pIII-scFv fusion protein. Quantification of the band intensities using the software package NIH image indicates that pre-selection results in an 11.8 fold increase in the amount of fusion protein present in the phage. Indeed, 43% of the total pIII in the pre-selected phage exists as pIII-scFv fusion, suggesting that most phage particles will have at least one scFv displayed on the surface.

Hence, not only does pre-selection using generic ligands enable enrichment of functional members from a repertoire but it also leads to preferential selection of those members which are well expressed and (if required) are able to elicit a high level of display on the surface of phage without being cleaved by bacterial proteases.

TABLE 1
PCR Primers for the Assembly of the “Primary” and “Somatic” Antibody Libraries

Template

1st round of amplification

| | | | |
|---|---|---|---|
| 1A | DP-47 | 5′(back) primer | GAGGTGCAGCTGTTGGAGTC |
| | | DVT 3′(forward) primer | GCCCTTCACGGAGTCTGCGTAMNNTGTMNNMNNACCMNNMNNMNNAA |
| | | | TMNNTGAGACCCACTCCAGCCC |
| | | NNK 3′(forward) primer | TABHTGAGACCCACTCCAGCCCGCCCTTCACGGAGTCTGCGTAABHT |
| | | | GTABHABHACCABHABHABHAA |
| 2A | DP-47 | 5′(back) primer | CGCAGACTCCGTGAAGGGC |
| | | DVT 3′(forward) primer | TCCCTGGCCCCAGTAGTCAAAMNNMNNMNNMNNTTTCGCACAGTAAT |
| | | | ATACGG |
| | | NNK 3′(forward) primer | TCCCTGGCCCCAGTAGTCAAAABHABHABHABHTTTCGCACAGTAAT |
| | | | ATACGG |
| 3A | DPK9 | 5′(back) primer | GACATCCAGATGACCCAGTC |
| | | DVT 3′(forward) primer | ATGGGACCCCACTTTGCAAMNNGGATGCMNNATAGATCAGGAGCTTA |
| | | | GGGG |
| | | NNK 3′(forward) primer | ATGGGACCCCACTTTGCAAABHGGATGCABHATAGATCAGGAGCTTA |
| | | | GGGG |
| 4A | DPK9 | 5′(back) primer | TTGCAAAGTGGGGTCCCAT |
| | | DVT 3′(forward) primer | CTTGGTCCCTTGGCCGAACGTMNNAGGMNNMNNMNNMNNCTGTTGAC |
| | | | AGTAGTAAGTTGC |
| | | NNK 3′(forward) primer | CTTGGTCCCTTGGCCGAACGTABHAGGABHABHABHABHCTGTTGAC |
| | | | AGTAGTAAGTTGC |
| 1B | DP-47 | 5′(back) primer | GAGGTGCAGCTGTTGGAGTC |
| | | DVT 3′(forward) primer | CTGGAGCCTGGCGGACCCAMNNCATMNNATAMNNGCTAAAGGTGAAT |
| | | | CCAGAG |
| | | NNK 3′(forward) primer | CTGGAGCCTGGCGGACCCAABHCATABHATAABHGCTAAAGGTGAAT |
| | | | CCAGAG |
| 2B | DP-47 | 5′(back) primer | TGGGTCCGCCAGGCTCCAG |
| | | DVT 3′(forward) primer | TCCCTGGCCCCAGTAGTCAAAMNNMNNMNNMNNTTTCGCACAGTAAT |
| | | | ATACGG |
| | | NNK 3′(forward) primer | TCCCTGGCCCCAGTAGTCAAAABHABHABHABHTTTCGCACAGTAAT |
| | | | ATACGG |
| 3B | DPK9 | 5′(back) primer | GACATCCAGATGACCCAGTC |
| | | DVT 3′(forward) primer | CTGGTTTCTGCTGATACCAMNNTAAMNNMNNMNNAATGCTCTGACTT |
| | | | GCCCGG |
| | | NNK 3′(forward) primer | CTGGTTTCTGCTGATACCAABHTAAABHABHABHAATGCTCTGACTT |
| | | | GCCCGG |
| 4B | DPK9 | 5′(back) primer | TGGTATCAGCAGAAACCAGGG |
| | | DVT 3′(forward) primer | CTTGGTCCCTTGGCCGAACGTMNNAGGGGTACTGTAACTCTGTTGAC |
| | | | AGTAGTAAGTTGC |
| | | NNK 3′(forward) primer | CTTGGTCCCTTGGCCGAACGTABHAGGGGTACTGTAACTCTGTTGAC |
| | | | AGTAGTAAGTTGC |

2nd round of amplification

| | | | |
|---|---|---|---|
| 5A | 1A/2A | 5′(back) primer | GTCCTCGCAACTGCGGCCCAGCCGGCCATGGCCGAGGTGCAGCTGTT |
| | | | GGAGTC |
| | | 3′(forward) primer | GAACCGCCTCCACCGCTCGAGACGGTGACCAGGGTTCCCTGGCCCCA |
| | | | GTAGTCAAA |
| 6A | 3A/4A | 5′(back) primer | AGCGGTGGAGGCGGTTCAGGCGGAGGTGGCAGCGGCGGTGGCGGGTC |
| | | | GACGGACATCCAGATGACCCAGTC |
| | | 3′(forward) primer | GAGTCATTCTCGACTTGCGGCCGCCCGTTTGATTTCCACCTTGGTCC |
| | | | CTTGGCCGAACG |
| 5B | 1B/2B | 5′(back) primer | GTCCTCGCAACTGCGGCCCAGCCGGCCATGGCCGAGGTGCAGCTGTT |
| | | | GGAGTC |
| | | 3′(forward) primer | GAACCGCCTCCACCGCTCGAGACGGTGACCAGGGTTCCCTGGCCCCA |
| | | | GTAGTCAAA |
| 6B | 3B/4B | 5′(back) primer | AGCGGTGGAGGCGGTTCAGGCGGAGGTGGCAGCGGCGGTGGCGGGTC |
| | | | GACGGACATCCAGATGACCCAGTC |
| | | 3′(forward) primer | GAGTCATTCTCGACTTGCGGCCGCCCGTTTGATTTCCACCTTGGTCC |
| | | | CTTGGCCGAACG |

It would be advantageous to be able to select unpaired or loosely paired antibody single variable domains from a population of antibody polypeptides. It would also be useful to be able to select unpaired or loosely paired T-cell receptor domains (V_{α} or V_{β}) from a population of T-cell receptor polypeptides. The present invention fulfils these needs.

In all embodiments and aspects of the invention described herein, where a target ligand is mentioned the target ligand selected from the group consisting of TNF alpha, serum albumin, von Willebrand's factor (vWF), IgE, interferon gamma, EGFR, IgE, MMP12, PDK1 and Amyloid beta (A-beta), or any one of the targets listed in Annex 1.

Reference is made to WO05044858A1, WO04062551A2, WO04041867A2, WO04041865A2, WO04041863A2, WO04041862A2, WO03050531A2 and EP0656946 for a description of Camelid VHH domains and Nanobodies™. These disclosures and definitions of VHH and Nanobodies™, as well as the sequences and examples disclosed, are specifically incorporated herein by reference.

For the methods of the invention, in one embodiment the population of polypeptides (eg, antibody polypeptides) is provided by B cells (eg, peripheral blood lymphocytes), wherein the B-cell population is provided in a plurality of wells or receptacles, each well or receptacle containing a single B-cell type or on average one B-cell type. Reference is made to de Wildt et al (1997) j. Immunol. Methods 2073, 61-67 and Babcook et al (1996) Proc. Natl. Acad. Sci. USA 93, 7843-7848 for disclosure of how to control the B cell average. These references are incorporated herein by reference in their entirety.

Specific Application of the Invention to Selecting B Cell Populations, e.g. from Camelids

The present invention provides in one aspect a selection system which eliminates (or significantly reduces the proportion of) antigen-specific B-cells (as a sub-population) which do not display the preferred antibody type whilst enriching for those antigen-specific B-cells (as a sub-population) which do display the preferred antibody type. This method of enrichment is carried out using a generic ligand, i.e. a protein or a small chemical molecule that has affinity for a structural feature common to all antigen-specific B-cells which do display the preferred antibody type and that is not common to all antigen-specific B-cells which do not display the preferred antibody type. For example, a generic ligand could have affinity to an antibody domain or an antibody surface patch that is common to all antigen-specific B-cells which do display the preferred antibody type and that is not common to all antigen-specific B-cells which do not display the preferred antibody type. Obviously, the selection with a generic ligand can be performed before, during or after selection for antigen-binding activity, the final outcome being a sub-population of antigen-specific B-cells expressing an antibody with desired type.

In the context of Camelidae B-cells displaying either conventional antibodies or heavy-chain antibodies, two methods of enrichment for one of the two sub-populations can be envisaged:

In the first approach, the superantigen protein G is of use in the present invention as generic ligand. Protein G binds to the CH1 constant domain in heavy chain. It has been shown (Hamers-Casterman et al. (1993) Nature, 363, 446-448) that a significant proportion of heavy-chain antibodies do not bind protein G (but well protein A) and this differential was used to separate conventional camel antibodies from heavy-chain camel antibodies by chromatographic means when analyzing animal sera. Obviously the deletion of the CH1 domain in heavy-chain antibodies is responsible for the absence of binding to immobilized protein G. In the present invention, protein G (preferably immobilised on a solid support) could be used to enrich for conventional antibody-expressing B-cells (these cells will bind to protein G) or to enrich for heavy-chain antibody-expressing B-cells (these cells will not bind to protein G). Alternatively, protein G could be labelled with a fluorescent dye (such as fluorescein, Cy3, Cy5, Texas Red to name a few) and incubated with the B-cell population. B-cell expressing conventional antibodies can then be separated from B-cells expressing heavy-chain antibodies by sorting for the different sub-populations on a Fluorescence-Activated Cell Sorter (FACS). Obviously, instead of protein G, one could use a monoclonal antibody or a polyclonal antiserum raised against the CH1 domain of camelidae antibodies. By extension to this approach, a monoclonal antibody or a polyclonal antiserum raised against the antibody light chain (either the light chain variable domain, the light chain constant domain or both) will also allow to separate the B-cell subpopulation expressing conventional domains from the B-cell sub-populations expressing heavy-chain antibodies.

In a second approach, a light chain variable domain is of use in the present invention. As described in the Introduction section of the present invention, variable light chain domains are always found in conventional antibodies as pairing with the heavy chain variable domains. In contrast heavy-chain antibodies do not have light chain variable domains. Thus on one hand—conventional antibodies—the VL binding site on the VH domain is occupied by a VL domain whereas in heavy chain antibodies the VL binding site on the VHH domain remains unoccupied. The VHH variable domains of Camelidae heavy chain have acquired mutations on the VL-binding site which are thought to prevent VL binding. However, it is worth pointing out that in VHH domains, these mutations (at positions 37, 44, 45 and 47—Kabat numbering) are not always identical and more importantly not always present in all positions at the same time. This degree of variability suggests that whilst this hallmark may be beneficial for biophysical properties such as solubility and monomeric state in solution, it can be advantageously replaced by others features such as a long CDR3, mutations at position W103 (Kabat numbering) and also amino acid compositions within the CDRs. Examples of such camel VHH sequences can be found in patents (WO 2004041862 tables 1, 4, 5, 6; WO 2004041865—table 4; WO 2004041863—tables 4, 5, 7, 8). Thus a significant proportion of camelid heavy chain variable domains present a former VL-interface akin to the VL-interface of heavy chain variable domains of conventional antibodies. This leads to the conclusion that such sub-population of B-cells can be separated from B-cells expressing conventional antibodies by contacting with an immobilized isolated VL domain (from Camelidae or from other mammals) or with a dye-labelled VL domain. By their combinatorial nature, VL and VH domains are promiscuous, enabling affinity maturation of conventional antibodies by chain shuffling (Marks et al. (1992) Biotechnology (NY) 10, 779-783). Therefore the likelihood of finding a promiscuous light chain variable domain for such selection is relatively good. It should also be noted that even if the monomeric interaction of a VL domain with a monomeric heavy-chain VHH is lower than that of a VL domain with a conventional VH domain, the proposed selection scheme would alleviate the problem. Indeed by immobilizing the VL domain and by contacting it with the B-cells, a large avidity effect will occur due to the display of many identical copies of antibodies on the surface of B-cells.

One interesting embodiment of this invention is the use of the VL domain to also differentiate between different types of conventional antibodies at the surface of B-cells. The affinity of a VL domain for a VH domain can vary (dissociation constant from 10⁻⁹ M to 10⁻⁶ M—reviewed by Plückthun (1992) Immunol. Rev. 130, 151-188). Therefore, even within conventional antibodies, one can anticipate that the presence of different sub-populations with different degrees of pairing strength between VL and VH domains. By using an immobilized VL domain for selection of B-cells displaying conventional antibodies, one could isolate those B-cells expressing conventional antibodies wherein the pairing between the VL and the VH domains within the antibody is weak but—because of the huge excess of immobilized VL domain, pairing of the VH domains with the immobilized VL domains will be encouraged thereby resulting in B-cell immobilisation. This approach is particularly interesting as it may help to select B-cell sub-populations that express conventional antibodies bearing highly promiscuous VH domains, a property of importance when reformatting of single variable domains is considered.

### I. Selection Using Antibody Light Chain Variable Domains

In one embodiment, the present invention provides a method for selecting, from a repertoire of antibody polypeptides, a population of functional variable domains which bind a target ligand and a generic ligand, which generic ligand is capable of binding functional members of the repertoire regardless of target ligand specificity, comprising the steps of:
a) contacting the repertoire with said generic ligand and selecting functional variable domains bound thereto; and
b) contacting the selected functional variable domains with the target ligand and selecting a population of variable domains which bind to the target ligand,
wherein the variable domains are heavy chain variable domains and the generic ligand is an antibody light chain variable domain.

The invention contemplates that the selected heavy chain variable domains can be present on IgG, Fab, Fab′, F(ab)₂, F(ab′)₂, scFv, Fv and a disulphide bonded Fv, i.e. an antibody or antibody fragment having at least one heavy and light chain variable domain pairing. Without wishing to be bound by any theory, it is believed that in some examples of these pairings, the variable domain pairings are loosely complementary, in that the domains may engage antigen predominantly through antigen binding exclusively or predominantly with one of the variable domains, and not the other in the pairing to any predominant extent. For example, in some VH/VL pairings, e.g. in some human or Camelid 4-chain IgG, the heavy chain variable domains may provide the predominant/exclusive binding contact with antigen. Thus, the present application has application to select for antibodies or antibody fragments of this description from the antibody polypeptide population, since the light chain variable domain used in step a) binds to at least one VH presented by antibodies or fragments (IgG, Fab, Fab′, F(ab)₂, F(ab′)₂, scFv, Fv or a disulphide bonded Fv) with loose VH/VL complementarity. This provides one with a useful way of selecting for VH that can be used as single variable domain (dAb or Nanobody™) diagnostic, therapeutic and/or prophylactic products, or starting points for the development of these. The selected antibody or antibody fragments themselves may have utility as such products, for example to address antigens where “breathability” or loose VH/VL pairing may be an advantage. Thus, for example the invention be used to make such a selection of such desirable IgG as a subset from a population of IgG, e.g. a population of Camel or human IgG or humanised or chimaeric IgG. As an extension of this concept, the inventions described herein in sections I and II can be used to select an antibody or antibody fragment (e.g. IgG, Fab, Fab′, F(ab)₂, F(ab′)₂, diabody, scFv paired dimer) that comprises a dual specific ligand as disclosed in any one of WO03002609A2, WO04003019A2 and WO04058821A2 where the dual specific ligand has at least one VH/VL or VH/VH or VL/VL pairing in which each variable domain in the pairing binds a respective antigen. The antigen species may be the same (e.g. VH and VL both binding TNF alpha, vWF, serum albumin or any other target antigen disclosed herein) or the variable domains may bind different antigen species, eg, one binding serum albumin and the other TNF alpha or any other target antigen.

In one embodiment, the population of antibody polypeptides is a population of antibody single variable domains. Thus, the population can be a population of Camelid or human antibody heavy chain variable domains. In one embodiment, it is a population of VHH domains or Nanobodies™.

Optionally the heavy chain variable domains in step b) are Camelid variable domains (VHH), Nanobodies™ or derived from a Camelid heavy chain antibody (H2 antibody); or optionally each heavy chain variable domain is a human variable domain or derived from a human.

Preferably, the repertoire of antibody polypeptides is first contacted with the target ligand and then with the generic ligand. Alternatively, the antibody polypeptides is contacted with the generic ligand and then with the target ligand.

Preferably, the generic ligand binds a subset of variable domains in the repertoire. In one example, two or more subsets of heavy chain variable domains are selected from the repertoire of polypeptides. The selection in this case may be performed with two or more generic ligands, i.e. two or more different light chain variable domains. Preferably, the two or more subsets are combined after selection to produce a further repertoire of polypeptides which can then be selected against a light chain variable domain according to the invention.

In one embodiment, two or more repertoires of polypeptides are contacted with generic ligands (the same or different generic ligands) and the subsets of polypeptides thereby obtained are then combined.

In another aspect of the invention, there is provided a method for selecting at least one antibody heavy chain variable domain from a population of antibody polypeptides, the method comprising:
a) contacting the population with an antibody light chain variable domain and
b) selecting at least one antibody heavy chain variable domain that binds to the light chain variable domain.

As with the embodiment above, it is contemplated that the selected heavy chain variable domains can be present on IgG, Fab, Fab′, F(ab)₂, F(ab′)₂, scFv, Fv and a disulphide bonded Fv, i.e. an antibody or antibody fragment having at least one heavy and light chain variable domain pairing. The discussion above on selecting VH from loose VH/VL pairings applies here too.

Preferably, prior to step a), there is a step of contacting antibody polypeptides with a target ligand and selecting antibody polypeptides that bind the target ligand, thereby providing said population of antibody polypeptides used in step a).

Preferably, after to step b), there is a step of contacting antibody heavy chain variable domains selected in step b) with a target ligand and selecting heavy chain variable domains that bind the target ligand.

Preferably, each heavy chain domain selected in step b) is from the group consisting of heavy chain variable domains derived from a Camelid; a VHH domain; a Nanobody™; a VHH having a glycine at position 44; a VHH having a leucine at position 45; a VHH having a tryptophan at position 47; a VHH having a glycine at position 44 and a leucine at position 45; a VHH having a glycine at position 44 and a tryptophan at position 47; a VHH having a leucine at position 45 and a tryptophan at position 47; a VHH having a glycine at position 44, a leucine at position 45 and a tryptophan at position 47; a VHH having a tryptophan or arginine at position 103. Numbering is according to Kabat numbering convention (Kabat et al., 1991, Sequences of Immunological Interest, 5^{th} ed. U.S. Dept. Health & Human Services, Washington, D.C.).

Preferably, each heavy chain domain selected in step b) is a humanised Camelid or murine heavy chain variable domain or a humanised Nanobody™.

Preferably, each heavy chain domain selected in step b) is a human heavy chain variable domain; a heavy chain variable domain derived from a human; or a humanised heavy chain variable domain (e.g. a humanised Camelid or murine variable domain).

Preferably, the light chain variable domain is a human light chain variable domain; derived from a human; a light chain variable domain having a FW2 sequence that is identical to FW2 encoded by germline gene sequence DPK9; or a light chain domain (e.g. a Camelid-derived VL) having a human interface region (i.e. the region usually interfacing with VH domains in human VH/VL pairings). In another example, the light chain variable domain is a Camelid light chain variable domain or derived from a Camelid.

Preferably, the population in step a) is provided by a population of B-cells, for example peripheral blood lymphocytes. In one example the B-cells are isolated from a mammal (e.g. a mouse or a Camelid, e.g. a llama or camel) that has been immunised with a target antigen. In another example, the B-cells are isolated from a mammal (e.g. a mouse or a Camelid, e.g. a llama or camel) that has not been immunised with a target antigen.

In an alternative, the population used in step a) is provided by a repertoire of antibody polypeptides encoded by synthetically rearranged antibody genes.

In an embodiment, the population used in step a) is provided by a phage display library comprising bacteriophage displaying said antibody polypeptides. Examples of such libraries are disclosed in WO99/20749. Reference is also made to WO04003019A2, WO05044858A1 WO04062551A2, WO04041867A2, WO04041865A2, WO04041863A2, and WO04041862A2 for examples of phage display libraries.

In an embodiment, the population used in step a) comprises (i) antibody polypeptides each comprising at least one heavy chain variable domain that is not paired with a light chain variable domain; and (ii) antibody polypeptides each comprising a heavy chain variable domain that is paired with a light chain variable domain. Thus, for example, the method of the invention is useful for selecting out antibody single variable domains (i.e. unpaired V domains) from a mixed population also comprising paired V domains, e.g. in the form of IgG. Thus, the invention finds utility in selecting VHH single variable domains from a mixed population (e.g. provided by B-cells such as peripheral blood lymphocytes) also comprising Camelid 4-chain IgG (which has paired VH/VL domains). Similarly, there is utility for selecting human VH from a mixed population also comprising human 4-chain IgG. If single variable domains are selected along with IgG in which the VH/VL pairings are “breathable” as described above, there may be an additional step after step b), wherein the single variable domains are separated from the selected IgG (eg, on the basis of size or by any other conventional technique).

The nucleotide sequence encoding a selected single variable domain using any embodiment of the invention can be isolated from the B-cell or phage (or yeast or any other system used in the library to connect phenotype to genotype) and inserted into an expression vector for expression of the variable domain. Optionally, the nucleotide sequence can be mutated (e.g. by introduction of one or more mutations in CDR3 and/or a FW) and/or operatively linked to one or more antibody domains (eg another single variable domain), an antibody Fc domain, a label or an effector group before expression from the expression vector.

Preferably, the population used in step a) comprises camelid heavy chain single variable domains (VHH) or Nanobodies™.

Preferably, the population used in step a) comprises human heavy chain single variable domains (VH).

In one particularly preferred embodiment, there is provided a method for selecting at least one Camelid antibody VHH domain from a population of Camelid antibody polypeptides provided by B-cells isolated from a Camelid that has been immunised with a target antigen, the method comprising:
a) contacting the population with an antibody light chain variable domain and
b) selecting at least one VHH domain that binds to the light chain variable domain.

In this preferred embodiment, preferably the light chain variable domain is a human light chain variable domain.

In this preferred embodiment, preferably the B-cells are provided in a plurality of wells or receptacles, wherein each well or receptacle contains on average one B-cell type.

The invention also provides a method comprising (i) using a target antigen to performing SLAM (selected lymphocyte antibody method) on a starting population of antibody polypeptides to select a population of antibody polypeptides that bind the target antigen; and (ii) using the selected population as the population of antibody polypeptides used in step a) of the method of the invention.

In the method of the invention, in step b) at least one of the selected antibody heavy chain variable domains is preferably fused or conjugated to a protein moiety. Preferably, the protein moiety is selected from a bacteriophage coat protein, one or more antibody domains, an antibody Fc domain, an enzyme, a toxin, a label and an effector group.

Preferably, in step b) at least one of the selected antibody heavy chain variable domains is part of an antibody or an antibody fragment selected from an IgG, Fab, Fab′, F(ab)₂, F(ab′)₂, scFv, Fv and a disulphide bonded Fv.

The present invention also provides an isolated antibody polypeptide comprising or consisting of an antibody heavy chain variable domain, wherein the polypeptide is obtainable by the method comprising steps a) and b), wherein the light chain variable domain in the method is a human light chain variable domain and the heavy chain variable domain is from a non-human mammal, e.g. Camelid. Preferably, the heavy chain variable domain is from the group consisting of a heavy chain variable domain derived from a Camelid; a VHH domain; a Nanobody™; a VHH having a glycine at position 44; a VHH having a leucine at position 45; a VHH having a tryptophan at position 47; a VHH having a glycine at position 44 and a leucine at position 45; a VHH having a glycine at position 44 and a tryptophan at position 47; a VHH having a leucine at position 45 and a tryptophan at position 47; a VHH having a glycine at position 44, a leucine at position 45 and a tryptophan at position 47; a VHH having a tryptophan or arginine at position 103.

Preferably, the heavy chain variable domain is provided as part of a Camelid IgG or an IgG derived from a Camelid.

Preferably, the heavy chain variable domain is provided as part of a human IgG or an IgG derived from a human, and wherein the heavy chain variable domain is paired in the IgG with a light chain variable domain that is different from the light chain variable domain used in step a) of the method.

The invention also provides the use of such an antibody polypeptide as a medicament.

The invention also provides the use of such an antibody polypeptide for therapy and/or prevention of a disease or condition in a human. Applicable conditions and diseases are disclosed in WO04003019A2, WO05044858A1, WO04062551A2, WO04041867A2, WO04041865A2, WO04041863A2, and WO04041862A2, as are routes of deliver, administration and formulation. All of these specific disclosures are explicitly incorporated into the present disclosure by reference as suitable examples for application to the present invention.

### II. Selection Using Antibody Heavy Chain Variable Domains

In one embodiment, the present invention provides a method for selecting, from a repertoire of antibody polypeptides, a population of functional variable domains which bind a target ligand and a generic ligand, which generic ligand is capable of binding functional members of the repertoire regardless of target ligand specificity, comprising the steps of:
a) contacting the repertoire with said generic ligand and selecting functional variable domains bound thereto; and
b) contacting the selected functional variable domains with the target ligand and selecting a population of variable domains which bind to the target ligand,
wherein the variable domains are light chain variable domains and the generic ligand is an antibody heavy chain variable domain.

The invention contemplates that the selected light chain variable domains can be present on IgG, Fab, Fab′, F(ab)₂, F(ab′)₂, scFv, Fv and a disulphide bonded Fv, i.e. an antibody or antibody fragment having at least one heavy and light chain variable domain pairing. Without wishing to be bound by any theory, it is believed that in some examples of these pairings, the variable domain pairings are loosely complementary, in that the domains may engage antigen predominantly through antigen binding exclusively or predominantly with one of the variable domains, and not the other in the pairing to any predominant extent. For example, in some VH/VL pairings, e.g. in some human or Camelid 4-chain IgG, the light chain variable domains may provide the predominant/exclusive binding contact with antigen. Thus, the present application has application to select for antibodies or antibody fragments of this description from the antibody polypeptide population, since the heavy chain variable domain used in step a) binds to at least one VL presented by antibodies or fragments (IgG, Fab, Fab′, F(ab)₂, F(ab′)₂, scFv, Fv or a disulphide bonded Fv) with loose VH/VL complementarity. This provides one with a useful way of selecting for VL that can be used as single variable domain (dAb or Nanobody™) diagnostic, therapeutic and/or prophylactic products, or starting points for the development of these. The selected antibody or antibody fragments themselves may have utility as such products, for example to address antigens where “breathability” or loose VH/VL pairing may be an advantage. Thus, for example the invention be used to make such a selection of such desirable IgG as a subset from a population of IgG, eg a population of Camel or human IgG or humanised or chimaeric IgG. As an extension of this concept, the inventions described herein in sections I and II can be used to select an antibody or antibody fragment (eg, IgG, Fab, Fab′, F(ab)₂, F(ab′)₂, diabody, scFv paired dimer) that comprises a dual specific ligand as disclosed in any one of WO03002609A2, WO04003019A2 and WO04058821A2 where the dual specific ligand has at least one VH/VL or VH/VH or VL/VL pairing in which each variable domain in the pairing binds a respective antigen. The antigen species may be the same (eg, VH and VL both binding TNF alpha, vWF, serum albumin or any other target antigen disclosed herein) or the variable domains may bind different antigen species, eg, one binding serum albumin and the other TNF alpha or any other target antigen.

In one embodiment, the population of antibody polypeptides is a population of antibody single variable domains. Thus, the population can be a population of Camelid or human antibody light chain variable domains.

Optionally the light chain variable domains in step b) are Camelid light domains, or derived from a Camelid; or optionally each light chain variable domain is a human variable domain or derived from a human.

Preferably, the repertoire of antibody polypeptides is first contacted with the target ligand and then with the generic ligand. Alternatively, the antibody polypeptides is contacted with the generic ligand and then with the target ligand.

Preferably, the generic ligand binds a subset of variable domains in the repertoire. In one example, two or more subsets of heavy chain variable domains are selected from the repertoire of polypeptides. The selection in this case may be performed with two or more generic ligands, i.e. two or more different heavy chain variable domains. Preferably, the two or more subsets are combined after selection to produce a further repertoire of polypeptides which can then be selected against a heavy chain variable domain according to the invention.

In one embodiment, two or more repertoires of polypeptides are contacted with generic ligands (the same or different generic ligands) and the subsets of polypeptides thereby obtained are then combined.

In another aspect of the invention, there is provided a method for selecting at least one antibody light chain variable domain from a population of antibody polypeptides, the method comprising:
a) contacting the population with an antibody heavy chain variable domain and
b) selecting at least one antibody light chain variable domain that binds to the heavy chain variable domain.

As with the embodiment above, it is contemplated that the selected light chain variable domains can be present on IgG, Fab, Fab′, F(ab)₂, F(ab′)₂, scFv, Fv and a disulphide bonded Fv, i.e. an antibody or antibody fragment having at least one heavy and light chain variable domain pairing. The discussion above on selecting VL from loose VH/VL pairings applies here too.

Preferably, prior to step a), there is a step of contacting antibody polypeptides with a target ligand and selecting antibody polypeptides that bind the target ligand, thereby providing said population of antibody polypeptides used in step a).

Preferably, after to step b), there is a step of contacting antibody light chain variable domains selected in step b) with a target ligand and selecting light chain variable domains that bind the target ligand.

Preferably, each light chain domain selected in step b) is a humanised Camelid or murine light chain variable domain.

Preferably, each light chain domain selected in step b) is a human light chain variable domain; a light chain variable domain derived from a human; or a humanised light chain variable domain (e.g. a humanised Camelid or murine variable domain).

Preferably, the heavy chain variable domain is a human heavy chain variable domain; derived from a human; a light chain variable domain having a FW2 sequence that is identical to FW2 encoded by germline gene sequence DP47; or a heavy chain variable domain having positions 44, 45 and 47 that are identical to positions 44, 45 and 47 encoded by germline gene sequence DP47; or a heavy chain domain (eg a Camelid-derived VH) having a human interface region (i.e. the region usually interfacing with VL domains in human VH/VL pairings). In another example, the heavy chain variable domain is a Camelid heavy chain variable domain or derived from a Camelid.

Preferably, the population in step a) is provided by a population of B-cells, for example peripheral blood lymphocytes. In one example the B-cells are isolated from a mammal (e.g. a mouse or a Camelid, e.g. a llama or camel) that has been immunised with a target antigen. In another example, the B-cells are isolated from a mammal (e.g. a mouse or a Camelid, e.g. a llama or camel) that has not been immunised with a target antigen.

In an alternative, the population used in step a) is provided by a repertoire of antibody polypeptides encoded by synthetically rearranged antibody genes.

In an embodiment, the population used in step a) is provided by a phage display library comprising bacteriophage displaying said antibody polypeptides. Examples of such libraries are disclosed in WO99/20749. Reference is also made to WO04003019A2, WO05044858A1, WO04062551A2, WO04041867A2, WO04041865A2, WO04041863A2, and WO04041862A2 for examples of phage display libraries.

In an embodiment, the population used in step a) comprises (i) antibody polypeptides each comprising at least one light chain variable domain that is not paired with a heavy chain variable domain; and (ii) antibody polypeptides each comprising a heavy chain variable domain that is paired with a light chain variable domain. Thus, for example, the method of the invention is useful for selecting out antibody single variable domains (i.e. unpaired V domains) from a mixed population also comprising paired V domains, e.g. in the form of IgG. Thus, the invention finds utility in selecting VL single variable domains from a mixed population (e.g. provided by B-cells such as peripheral blood lymphocytes) also comprising Camelid or human 4-chain IgG (which has paired VH/VL domains). If single variable domains are selected along with IgG in which the VH/VL pairings are “breathable” as described above, there may be an additional step after step b), wherein the single variable domains are separated from the selected IgG (e.g. on the basis of size or by any other conventional technique).

The nucleotide sequence encoding a selected single variable domain using any embodiment of the invention can be isolated from the B-cell or phage (or yeast or any other system used in the library to connect phenotype to genotype) and inserted into an expression vector for expression of the variable domain. Optionally, the nucleotide sequence can be mutated (e.g. by introduction of one or more mutations in CDR3 and/or a FW) and/or operatively linked to one or more antibody domains (e.g. another single variable domain), an antibody Fc domain, a label or an effector group before expression from the expression vector.

Preferably, the population used in step a) comprises camelid light chain single variable domains.

Preferably, the population used in step a) comprises human light chain single variable domains (VL).

The invention also provides a method comprising (i) using a target antigen to performing SLAM (selected lymphocyte antibody method) on a starting population of antibody polypeptides to select a population of antibody polypeptides that bind the target antigen; and (ii) using the selected population as the population of antibody polypeptides used in step a) of the method of the invention.

In the method of the invention, in step b) at least one of the selected antibody light chain variable domains is preferably fused or conjugated to a protein moiety. Preferably, the protein moiety is selected from a bacteriophage coat protein, one or more antibody domains, an antibody Fc domain, an enzyme, a toxin, a label and an effector group. Preferably, in step b) at least one of the selected antibody light chain variable domains is part of an antibody or an antibody fragment selected from an IgG, Fab, Fab′, F(ab)₂, F(ab′)₂, scFv, Fv and a disulphide bonded Fv.

The present invention also provides an isolated antibody polypeptide comprising or consisting of an antibody light chain variable domain, wherein the polypeptide is obtainable by the method comprising steps a) and b), wherein the heavy chain variable domain in the method is a human heavy chain variable domain and the heavy chain variable domain is from a non-human mammal, e.g. a Camelid. Preferably, the light chain variable domain is derived from a Camelid. Preferably, the light chain variable domain is provided as part of a Camelid IgG or an IgG derived from a Camelid.

Preferably, the light chain variable domain is provided as part of a human IgG or an IgG derived from a human, and wherein the light chain variable domain is paired in the IgG with a heavy chain variable domain that is different from the heavy chain variable domain used in step a) of the method.

The invention also provides the use of such an antibody polypeptide as a medicament.

The invention also provides the use of such an antibody polypeptide for therapy and/or prevention of a disease or condition in a human. Applicable conditions and diseases are disclosed in WO04003019A2, WO05044858A1, WO04062551A2, WO04041867A2, WO04041865A2, WO04041863A2, and WO04041862A2, as are routes of deliver, administration and formulation. All of these specific disclosures are explicitly incorporated into the present disclosure by reference as suitable examples for application to the present invention.

In one embodiment, a population of antibody heavy chain variable domains are selected according to a method set out in Section I and a population of antibody light chain variable domains is selected according to a method set out in Section II and the populations thereby obtained are then combined.

### III. Selection Using T-Cell Receptor Domains

The present invention provides a method for selecting, from a repertoire of polypeptides, a population of functional T-cell receptor domains which bind a target ligand and a generic ligand, which generic ligand is capable of binding functional members of the repertoire regardless of target ligand specificity, comprising the steps of:
a) contacting the repertoire with said generic ligand and selecting functional T-cell receptor domains bound thereto; and
b) contacting the selected functional T-cell receptor domains with the target ligand and selecting a population of T-cell receptor domains which bind to the target ligand,
wherein either a) the T-cell receptor domains are V_{α} domains and the generic ligand is a T-cell receptor V_{β} domain; or b) the T-cell domains are T-cell receptor V_{β} domains and the generic ligand is a T-cell receptor V_{α} domain.

Optionally in a) the T-cell receptor V_{α} domains are Camelid domains derived from a Camelid; or optionally in a) and b) each T-cell receptor domain is a human domain or derived from a human.

Preferably, a population of T-cell receptor V_{α} domains is selected according to the method and a population of T-cell receptor V_{β} domains is selected according to the method and the populations thereby obtained are then combined.

### FURTHER EXAMPLES

### Example A1

### Isolating Variable Domains of Antigen-Specific Heavy-Chain Antibodies from Immunized Llama in Two Biopanning Rounds

One adult llama will be injected at days 0, 7, 14, 21, 28, 35, 42, 49, and 54 with 1 mg human tetanus toxoid (TT). Serum will be collected prior to each injection to follow the immune response against the immunogen. Anticoagulated blood (150 ml) will be collected from the immunized animal and peripheral blood lymphocytes (PBLs) will be prepared with Unisep (WAK Chemie, Germany).

Sterile ELISA plates will be coated with protein G (10 μg/ml overnight at 4° C.), then washed with sterile PBS, blocked with sterile PBS-10% IgG-depleted FCS (foetal calf serum) and then washed again with PBS.

Sterile ELISA plates will be coated with TT (10 μg/ml overnight at 4° C.), then washed with sterile PBS, blocked with sterile PBS-10% IgG-depleted FCS (foetal calf serum) and then washed again with PBS.

The purified PBLs (in PBS) will be first added to the protein G-coated wells and allowed to bind for at least one hour at 37° C. Unbound cells (mainly displaying heavy-chain antibodies or no antibodies) in the supernatant will be carefully removed from each protein G-coated well and will be combined.

The cell population enriched in heavy-chain-expressing B-cells will be added to the TT-coated wells (at a density of 300 cells per well and 1,500 cell per well) and allowed to bind for at least one hour at 37° C. Unbound cells in the supernatant will be removed by washing ten times with culture media. The remaining cells expressing antigen-specific heavy-chain antibodies will be cultured in the presence of coated antigen, T cell conditioned media (3%) and EL-4 cells (5×10⁴/well) for seven days.

Positive wells secreting antigen-specific antibodies will be identified by analyzing a small volume of culture supernatant for antibody binding in fresh TT-coated wells by ELISA using protein A-horseradish peroxidase conjugate and TMB substrate.

Positive B-cell wells will be selected for further processing: the B-cells will be pelleted, supernatant will be removed and the cell pellet will be resuspended in 10 μl of fresh media (DMEM or RPMI with 1-6% T-cell conditioned medium). Aliquots (2 μl each) will be taken for PCR using the MJ Research Robus RT-PCR kit (Catalogue No. F-580L) using the following mix per tube to isolate the heavy chain variable domains:

| DEPC water | 35.5 | μl |
|---|---|---|
| 10xbuffer | 5 | μl |
| dNTPS | 1 | μl |
| 10% NP-40 | 2.5 | μl |
| RNAsin | 0.5 | μl |
| RT | 1 | μl |
| Polymerase | 2 | μl |
| Primer mix (*) | 1 | μl |
| | (10 μM each) | |
| MgCl2 | 1.5 | |

PCR program: 30 min at 50° C., followed 2 min at 94° C. and then 40 cycles of [94° C./1 min, 55° C./1 min, 72° C./1 min]. Final extension: 72° C./5 min
(*): The mix will contain the following two primers: an oligo-dT primer and a single degenerated FRI primer: 5′-GAGGTBCARCTGCAGGASTCYGG-3′ which encodes a PstI restriction site.

The 1,300 by fragment will be cleaved with PstI and BstEII. The latter enzyme frequently cleaves in the DNA segment encoding framework 4 of heavy-chain antibodies.

The digested product will be isolated from an agarose gel after electrophoresis and UV illumination and will be ligated into corresponding sites of a cloning vector that can be propagated in E. coli under selective antibiotic conditions for growth.

The ligated vector will be used to transform E. coli cells and the PstI/BstEII insert will be sequenced to reveal the DNA sequence of the heavy-chain variable domain(s).

### Example B

### Isolating Variable Domains of Antigen-Specific Heavy-Chain Antibodies from Immunized Llama by Biopanning and Flow Cytometry

One adult llama will be injected at days 0, 7, 14, 21, 28, 35, 42, 49, and 54 with 1 mg human tetanus toxoid (TT). Serum will be collected prior to each injection to follow the immune response against the immunogen. Anticoagulated blood (150 ml) will be collected from the immunized animal and peripheral blood lymphocytes (PBLs) will be prepared with Unisep (WAK Chemie, Germany).

Sterile ELISA plates will be coated with a VL domain (eg. the VL domain described in Conrath et al. (2005) J. Mol Biol. 350, 112-25) (10 μg/ml overnight at 4° C.), then washed with sterile PBS, blocked with sterile PBS-10% IgG-depleted FCS (foetal calf serum) and then washed again with PBS.

Tetanus toxoid will be labelled with Texas Red using N-hydrosuccinimidyl group (NHS) specific for the amino group of exposed lysine residues or with fluorescein-isothiocyanate (FITC). The excess fluorescent label will be removed by passing the labelled TT on a PD10 column (Amersham Biosciences). The extent of labelling with Texas Red or FITC will be evaluated by mass spectrometry (MALDI-TOF).

The purified PBLs will be first incubated with labelled TT for 30 min at room temperature (1 ug labelled n for 10⁶ cells). Optional step: the cells will be pelleted by centrifugation for 5 min at 200 g and resuspended in 0.5 ml PBS. The cells will then be sorted for TT-positive cells in a flow cytometer (eg. Coulter Epics Elite flow cytometer from Coulter, Fla., USA) equipped with an automatic cell deposit unit. All positive cells for TT-binding will be collected in a single receptacle.

At this stage, the antigen-specific B-cells will either express conventional antibodies or heavy-chain antibodies on the cell surface. Further separation for the sub-population of B-cells expressing antigen-specific heavy-chain antibodies will be performed as follows.

The combined B-cell population will be added to the protein L-coated wells and allowed to bind for at least one hour at 37° C. Unbound cells (mainly displaying conventional antibodies or no antibodies) in the supernatant will be removed by washing ten times with culture media. Bound cells (expressing antigen-specific heavy-chain antibodies) will be dislodged, counted and used to seed fresh EL4-B5 T-cells coated wells (5×10⁴/well) in the presence of T cell conditioned media (3%) at a seeding ratio of 0.3 cell per well. The cells will be cultured for 7 days after which positive wells secreting antigen-specific heavy-chain antibodies will be identified by analyzing a small volume of culture supernatant for antibody binding in fresh TT-coated wells by ELISA using protein A-horseradish peroxidase conjugate and TMB substrate.

Positive B-cell wells will be selected for further processing: the B-cells will be pelleted, supernatant will be removed and the cell pellet will be resuspended in 10 μl of fresh media (DMEM or RPMI with 1-6% T-cell conditioned medium). Aliquots (2 μl each) will be taken for PCR using the MJ Research Robus RT-PCR kit (Catalogue No. F-580L) using the following mix per tube to isolate the heavy chain variable domains.

| DEPC water | 35.5 | μl |
|---|---|---|
| 10xbuffer | 5 | μl |
| dNTPS | 1 | μl |
| 10% NP-40 | 2.5 | μl |
| RNAsin | 0.5 | μl |
| RT | 1 | μl |
| Polymerase | 2 | μl |
| Primer mix (*) | 1 | μl |
| | (10 μM each) | |
| MgCl2 | 1.5 | |

PCR program: 30 min at 50° C., followed 2 min at 94° C. and then 40 cycles of [94° C./1 min, 55° C./1 min, 72° C./1 min]. Final extension: 72° C./5 min
(*): The mix will contain the following two primers: an oligo-dT primer and a single degenerated FRI primer: 5′-GAGGTBCARCTGCAGGASTCYGG-3′ which encodes a PstI restriction site.

The 1,300 by fragment will be cleaved with PstI and BstEII. The latter enzyme frequently cleaves in the DNA segment encoding framework 4 of heavy-chain antibodies.

The digested product will be isolated from an agarose gel after electrophoresis and UV illumination and will be ligated into corresponding sites of a cloning vector that can be propagated in E. coli under selective antibiotic conditions for growth.

The ligated vector will be used to transform E. coli cells and the PstI/BstEII insert will be sequenced to reveal the DNA sequence of the heavy-chain variable domain(s).

### Annex 1

a) Cytokines, cytokine receptors, enzymes etc, including Cytokines, cytokine receptors, enzymes, enzyme co-factors, or DNA binding proteins. Suitable cytokines and growth factors include but are not limited to: ApoE, Apo-SAA, BDNF, Cardiotrophin-1, EGF, EGF receptor, ENA-78, Eotaxin, Eotaxin-2, Exodus-2, FGF-acidic, FGF-basic, fibroblast growth factor-10, FLT3 ligand, Fractalkine (CX3C), GDNF, G-CSF, GM-CSF, GF-β 1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (ME), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TACE recognition site, TGF-α, TGF-1β, TGF-β2, TGF-β3, tumor necrosis factor (TNF), TNF-α, TNF-β, TNF receptor I (p55), TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3 and HER 4. Cytokine receptors include receptors for each of the foregoing cytokines, e.g., IL-1R, IL-6R, IL-10R, IL-18R, etc. It will be appreciated that this list is by no means exhaustive. Preferred targets for antigen single variable domain polypeptides according to the invention are disclosed in WO04/041867 (the contents of which are incorporated herein in their entirety) and include, but are not limited to TNFα, IgE, IFNγ, MMP-12, EGFR, CEA, H. pylori, TB, influenza, PDK-1, GSK1, Bad, caspase, Forkhead and VonWillebrand Factor (vWF). Targets may also be fragments of the above targets. Thus, a target is also a fragment of the above targets capable of eliciting an immune response. A target is also a fragment of the above targets, capable of binding to an antibody single variable domain polypeptide raised against the full length target.

b) an antigen capable of increasing the half life of a moiety (the latter moiety being e.g. therapeutic or imaging protein moiety, e.g. an antibody or antibody fragment, e.g. an antibody variable domain such as a human VH or VL domain or a Camelid VHH)

### Alpha-1 Glycoprotein (Orosomucoid) (AAG)

### Alpha-1 Antichyromotrypsin (ACT)

### Alpha-1 Antitrypsin (AAT)

### Alpha-1 Microglobulin (Protein HC) (AIM)

### Alpha-2 Macroglobulin (A2M)

### Antithrombin III (AT III)

### Apolipoprotein A-1 (Apo A-1)

### Apolipoprotein B (Apo B)

Beta-2-microglobulin (B2M)

### Ceruloplasmin (Cp)

### Complement Component (C3)

### Complement Component (C4)

### C1 Esterase Inhibitor (C1 INH)

### C-Reactive Protein (CRP)

### Cystatin C (Cys C)

### Ferritin (FER)

### Fibrinogen (FIB)

### Fibronectin (FN)

### Haptoglobulin (Hp)

### Hemopexin (HPX)

### Immunoglobulin A (IgA)

### Immunoglobulin D (IgD)

### Immunoglobulin E (IgE)

### Immunoglobulin G (IgG)

### Immunoglobulin M (IgM)

Immunoglobulin Light Chains (kapa/lambda)

### Lipoproteina) [Lpa)]

Mannose-binding protein (MBP)

### Myoglobulin (Myo)

### Plasminogen (PSM)

### Prealbumin (Transthyretin) (PAL)

Retinol-binding protein (RBP)

### Rheomatoid Factor (RF)

### Serum Amyloid A (SAA)

Soluble Tranferrin Receptor (sTfR)

### Transferrin (Tf)

### G: Antigens Capable of Increasing Ligand Half-Life

Proteins from the extracellular matrix; for example collagen, laminins, integrins and fibronectin. Collagens are the major proteins of the extracellular matrix. About 15 types of collagen molecules are currently known, found in different parts of the body, e.g. type I collagen (accounting for 90% of body collagen) found in bone, skin, tendon, ligaments, cornea, internal organs or type II collagen found in cartilage, invertebral disc, notochord, vitreous humour of the eye.

Proteins found in blood, including:

Plasma proteins such as fibrin, α-2 macroglobulin, serum albumin, fibrinogen A, fibrinogen B. serum amyloid protein A, heptaglobulin, protein, ubiquitin, uteroglobulin and p-2-microglobulin;
Enzymes and inhibitors such as plasminogen, lysozyme, cystatin C, alpha-1-antitrypsin and pancreatic kypsin inhibitor. Plasminogen is the inactive precursor of the trypsin-like 2s serine protease plasmin. It is normally found circulating through the blood stream. When plasminogen becomes activated and is converted to plasmin, it unfolds a potent enzymatic domain that dissolves the fibrinogen fibres that entangle the blood cells in a blood clot. This is called fibrinolysis.

Immune system proteins, such as IgE, IgG, IgM.

Transport proteins such as retinol binding protein, o-1 microglobulin.

Defensins such as beta-defensin 1, Neutrophil defensins 1, 2 and 3.

Proteins found at the blood brain barrier or in neural tissues, such as melanocortin receptor, myelin, ascorbate transporter.

Transferrin receptor specific ligand-neuropharmaceutical agent fusion proteins (see U.S. Pat. No. 5,977,307);

Brain capillary endothelial cell receptor, transferrin, transferrin receptor, insulin, insulin-like growth factor 1 (IGF 1) receptor, insulin-like growth factor 2 (IGF 2) receptor, insulin receptor.

Proteins localised to the kidney, such as polycystin, type IV collagen, organic anion transporter K1, Heymann's antigen.

Proteins localised to the liver, for example alcohol dehydrogenase, G250.

Blood coagulation factor X
α1 antitrypsin

### HNF 1α

Proteins localised to the lung, such as secretory component (binds IgA).

Proteins localised to the Heart, for example HSP 27. This is associated with dilated cardiomyopathy.

Proteins localised to the skin, for example keratin.

Bone specific proteins, such as bone morphogenic proteins (BMPs), which are a subset of the transforming growth factor β superfamily that demonstrate osteogenic activity. Examples include BMP-2, -4, -5, -6, -7 (also referred to as osteogenic protein (OP-1) and -8 (OP-2).

Tumour specific proteins, including human trophoblast antigen, herceptin receptor, oestrogen receptor, cathepsins eg cathepsin B (found in liver and spleen).

Disease-specific proteins, such as antigens expressed only on activated T-cells: including LAG-3 (lymphocyte activation gene), osteoprotegerin ligand (OPGL) see Nature 402, 304-309; 1999, OX40 (a member of the TNF receptor family, expressed on activated T cells and the only costimulatory T cell molecule known to be specifically up-regulated in human T cell leukaemia virus type-I (HTLV-I)-producing cells.) See J Immunol. 2000 Jul. 1; 165(1):263-70; Metalloproteases (associated with arthritis/cancers), including CG6512 Drosophila, human paraplegia, human FtsH, human AFG3L2, murine ftsH; angiogenic growth factors, including acidic fibroblast growth factor (FGF-1), basic fibroblast growth factor (FGF-2), Vascular endothelial growth factor/vascular permeability factor (VEGF/VPF), transforming growth factor-a (TGF a), tumor necrosis factor-alpha (TNF-α), angiogenin, interleukin-3 (IL-3), interleukin-8 (IL-8), platelet-derived endothelial growth factor (PD-ECGF), placental growth factor (PlGF), midkine platelet-derived growth factor-BB (PDGF), fractalkine.

### Stress Proteins (Heat Shock Proteins)

HSPs are normally found intracellularly. When they are found extracellularly, it is an indicator that a cell has died and spilled out its contents. This unprogrammed cell death (necrosis) only occurs when as a result of trauma, disease or injury and therefore in vivo, extracellular HSPs trigger a response from the immune system that will fight infection and disease. A dual specific which binds to extracellular HSP can be localised to a disease site.

Proteins involved in Fc transport
Brambell receptor (also known as FcRB)

This Fc receptor has two functions, both of which are potentially useful for delivery.

The functions are:
1) the transport of IgG from mother to child across the placenta
2) the protection of IgG from degradation thereby prolonging its serum half life of IgG. It is thought that the receptor recycles IgG from endosome.

See Holliger et al, Nat Biotechnol 1997 July; 15(7):632-6.

c) a target antigen can also be any one of the antigens in the following table, irrespective of whether or not the antigen is shown in a pairing in the table:—

| Pairing | Therapeutic relevant references. |
|---|---|
| TNF | TGF-b and TNF when injected into the ankle joint of collagen |
| ALPHA/TGF-β | induced arthritis model significantly enhanced joint |
| | inflammation. In non-collagen challenged mice there was no |
| | effect. |
| TNF | TNF and IL-1 synergize in the pathology of uveitis. |
| ALPHA/IL-1 | TNF and IL-1 synergize in the pathology of malaria |
| | (hypoglycaemia, NO). |
| | TNF and IL-1 synergize in the induction of |
| | polymorphonuclear (PMN) cells migration in inflammation. |
| | IL-1 and TNF synergize to induce PMN infiltration into the |
| | peritoneum. |
| | IL-1 and TNF synergize to induce the secretion of IL-1 by |
| | endothelial cells. Important in inflammation. |
| | IL-1 or TNF alone induced some cellular infiltration into knee |
| | synovium. IL-1 induced PMNs, TNF - monocytes. Together |
| | they induced a more severe infiltration due to increased PMNs. |
| | Circulating myocardial depressant substance (present in sepsis) |
| | is low levels of IL-1 and TNFacting synergistically. |
| TNF | Most relating to synergistic activation of killer T-cells. |
| ALPHA/IL-2 | |
| TNF | Synergy of interleukin 3 and tumor necrosis factor alpha in |
| ALPHA/IL-3 | stimulating clonal growth of acute myelogenous leukemia |
| | blasts is the result of induction of secondary hematopoietic |
| | cytokines by tumor necrosis factor alpha. |
| | Cancer Res. 1992 Apr 15; 52(8): 2197-201. |
| TNF | IL-4 and TNF synergize to induce VCAM expression on |
| ALPHA/IL-4 | endothelial cells. Implied to have a role in asthma. Same for |
| | synovium - implicated in RA. |
| | TNF and IL-4 synergize to induce IL-6 expression in |
| | keratinocytes. |
| | Sustained elevated levels of VCAM-1 in cultured fibroblast- |
| | like synoviocytes can be achieved by TNF-alpha in |
| | combination with either IL-4 or IL-13 through increased |
| | mRNA stability. Am J Pathol. 1999 Apr; 154(4): 1149-58 |
| TNF | Relationship between the tumor necrosis factor system and the |
| ALPHA/IL-5 | serum interleukin-4, interleukin-5, interleukin-8, eosinophil |
| | cationic protein, and immunoglobulin E levels in the bronchial |
| | hyperreactivity of adults and their children. Allergy Asthma |
| | Proc. 2003 Mar-Apr; 24(2): 111-8. |
| TNF | TNF and IL-6 are potent growth factors for OH-2, a novel |
| ALPHA/IL-6 | human myeloma cell line. Eur J Haematol. 1994 Jul; 53(1): 31-7. |
| TNF | TNF and IL-8 synergized with PMNs to activate platelets. |
| ALPHA/IL-8 | Implicated in Acute Respiratory Distress Syndrome. |
| | See IL-5/TNF (asthma). Synergism between interleukin-8 and |
| | tumor necrosis factor-alpha for neutrophil-mediated platelet |
| | activation. Eur Cytokine Netw. 1994 Sep-Oct; 5(5): 455-60. |
| | (adult respiratory distress syndrome (ARDS)) |
| TNF | |
| ALPHA/IL-9 | |
| TNF | IL-10 induces and synergizes with TNF in the induction of |
| ALPHA/IL-10 | HIV expression in chronically infected T-cells. |
| TNF | Cytokines synergistically induce osteoclast differentiation: |
| ALPHA/IL-11 | support by immortalized or normal calvarial cells. Am J |
| | Physiol Cell Physiol. 2002 Sep; 283(3): C679-87. (Bone loss) |
| TNF | |
| ALPHA/IL-12 | |
| TNF | Sustained elevated levels of VCAM-1 in cultured fibroblast- |
| ALPHA/IL-13 | like synoviocytes can be achieved by TNF-alpha in |
| | combination with either IL-4 or IL-13 through increased |
| | mRNA stability. Am J Pathol. 1999 Apr; 154(4): 1149-58. |
| | Interleukin-13 and tumour necrosis factor-alpha synergistically |
| | induce eotaxin production in human nasal fibroblasts. Clin Exp |
| | Allergy. 2000 Mar; 30(3): 348-55. |
| | Interleukin-13 and tumour necrosis factor-alpha synergistically |
| | induce eotaxin production in human nasal fibroblasts. Clin Exp |
| | Allergy. 2000 Mar; 30(3): 348-55 (allergic inflammation) |
| | Implications of serum TNF-beta and IL-13 in the treatment |
| | response of childhood nephrotic syndrome. Cytokine. 2003 |
| | Feb 7; 21(3): 155-9. |
| TNF | Effects of inhaled tumour necrosis factor alpha in subjects with |
| ALPHA/IL-14 | mild asthma. Thorax. 2002 Sep; 57(9): 774-8. |
| TNF | Effects of inhaled tumour necrosis factor alpha in subjects with |
| ALPHA/IL-15 | mild asthma. Thorax. 2002 Sep; 57(9): 774-8. |
| TNF | Tumor necrosis factor-alpha-induced synthesis of interleukin- |
| ALPHA/IL-16 | 16 in airway epithelial cells: priming for serotonin stimulation. |
| | Am J Respir Cell Mol Biol. 2003 Mar; 28(3): 354-62. (airway |
| | inflammation) |
| | Correlation of circulating interleukin 16 with proinflammatory |
| | cytokines in patients with rheumatoid arthritis. Rheumatology |
| | (Oxford). 2001 Apr; 40(4): 474-5. No abstract available. |
| | Interleukin 16 is up-regulated in Crohn's disease and |
| | participates in TNBS colitis in mice. Gastroenterology. 2000 |
| | Oct; 119(4): 972-82. |
| TNF | Inhibition of interleukin-17 prevents the development of |
| ALPHA/IL-17 | arthritis in vaccinated mice challenged with Borrelia |
| | burgdorferi. Infect Immun. 2003 Jun; 71(6): 3437-42. |
| | Interleukin 17 synergises with tumour necrosis factor alpha to |
| | induce cartilage destruction in vitro. Ann Rheum Dis. 2002 |
| | Oct; 61(10): 870-6. |
| | A role of GM-CSF in the accumulation of neutrophils in the |
| | airways caused by IL-17 and TNF-alpha. Eur Respir J. 2003 |
| | Mar; 21(3): 387-93. (Airway inflammation) |
| | Abstract Interleukin-1, tumor necrosis factor alpha, and |
| | interleukin-17 synergistically up-regulate nitric oxide and |
| | prostaglandin E2 production in explants of human |
| | osteoarthritic knee menisci. Arthritis Rheum. 2001 |
| | Sep; 44(9): 2078-83. |
| TNF | Association of interleukin-18 expression with enhanced levels |
| ALPHA/IL-18 | of both interleukin-1beta and tumor necrosis factor alpha in |
| | knee synovial tissue of patients with rheumatoid arthritis. |
| | Arthritis Rheum. 2003 Feb; 48(2): 339-47. |
| | Abstract Elevated levels of interleukin-18 and tumor necrosis |
| | factor-alpha in serum of patients with type 2 diabetes mellitus: |
| | relationship with diabetic nephropathy. Metabolism. 2003 |
| | May; 52(5): 605-8. |
| TNF | Abstract IL-19 induces production of IL-6 and TNF-alpha and |
| ALPHA/IL-19 | results in cell apoptosis through TNF-alpha. J Immunol. 2002 |
| | Oct 15; 169(8): 4288-97. |
| TNF | Abstract Cytokines: IL-20-a new effector in skin |
| ALPHA/IL-20 | inflammation. Curr Biol. 2001 Jul 10; 11(13): R531-4 |
| TNF | Inflammation and coagulation: implications for the septic |
| ALPHA/Complement | patient. Clin Infect Dis. 2003 May 15; 36(10): 1259-65. Epub |
| | 2003 May 08. Review. |
| TNF | MHC induction in the brain. |
| ALPHA/IFN-γ | Synergize in anti-viral response/IFN□ induction. |
| | Neutrophil activation/respiratory burst. |
| | Endothelial cell activation |
| | Toxicities noted when patients treated with TNF/IFN-□ as |
| | anti-viral therapy |
| | Fractalkine expression by human astrocytes. |
| | Many papers on inflammatory responses - i.e. LPS, also |
| | macrophage activation. |
| | Anti-TNF and anti-IFN-γ synergize to protect mice from lethal |
| | endotoxemia. |
| TGF-β/IL-1 | Prostaglndin synthesis by osteoblasts |
| | IL-6 production by intestinal epithelial cells (inflammation model) |
| | Stimulates IL-11 and IL-6 in lung fibroblasts (inflammation |
| | model) |
| | IL-6 and IL-8 production in the retina |
| TGF-β/IL-6 | Chondrocarcoma proliferation |
| IL-1/IL-2 | B-cell activation |
| | LAK cell activation |
| | T-cell activation |
| | IL-1 synergy with IL-2 in the generation of lymphokine |
| | activated killer cells is mediated by TNF-alpha and beta |
| | (lymphotoxin). Cytokine. 1992 Nov; 4(6): 479-87. |
| IL-1/IL-3 | |
| IL-1/IL-4 | B-cell activation |
| | IL-4 induces IL-1 expression in endothelial cell activation. |
| IL-1/IL-5 | |
| IL-1/IL-6 | B cell activation |
| | T cell activation (can replace accessory cells) |
| | IL-1 induces IL-6 expression |
| | C3 and serum amyloid expression (acute phase response) |
| | HIV expression |
| | Cartilage collagen breakdown. |
| IL-1/IL-7 | IL-7 is requisite for IL-1-induced thymocyte proliferation. |
| | Involvement of IL-7 in the synergistic effects of granulocyte- |
| | macrophage colony-stimulating factor or tumor necrosis factor |
| | with IL-1. J Immunol. 1992 Jan 1; 148(1): 99-105. |
| IL-1/IL-8 | |
| IL-1/IL-10 | |
| IL-1/IL-11 | Cytokines synergistically induce osteoclast differentiation: |
| | support by immortalized or normal calvarial cells. Am J |
| | Physiol Cell Physiol. 2002 Sep; 283(3): C679-87. (Bone loss) |
| IL-1/IL-16 | Correlation of circulating interleukin 16 with proinflammatory |
| | cytokines in patients with rheumatoid arthritis. Rheumatology |
| | (Oxford). 2001 Apr; 40(4): 474-5. No abstract available. |
| IL-1/IL-17 | Inhibition of interleukin-17 prevents the development of |
| | arthritis in vaccinated mice challenged with Borrelia |
| | burgdorferi. Infect Immun. 2003 Jun; 71(6): 3437-42. |
| | Contribution of interleukin 17 to human cartilage degradation |
| | and synovial inflammation in osteoarthritis. Osteoarthritis |
| | Cartilage. 2002 Oct; 10(10): 799-807. |
| | Abstract Interleukin-1, tumor necrosis factor alpha, and |
| | interleukin-17 synergistically up-regulate nitric oxide and |
| | prostaglandin E2 production in explants of human |
| | osteoarthritic knee menisci. Arthritis Rheum. 2001 |
| | Sep; 44(9): 2078-83. |
| IL-1/IL-18 | Association of interleukin-18 expression with enhanced levels |
| | of both interleukin-1beta and tumor necrosis factor alpha in |
| | knee synovial tissue of patients with rheumatoid arthritis. |
| | Arthritis Rheum. 2003 Feb; 48(2): 339-47. |
| IL-1/IFN-g | |
| IL-2/IL-3 | T-cell proliferation |
| | B cell proliferation |
| IL-2/IL-4 | B-cell proliferation |
| | T-cell proliferation |
| | (selectively inducing activation of CD8 and NK |
| | lymphocytes)IL-2R beta agonist P1-30 acts in synergy with |
| | IL-2, IL-4, IL-9, and IL-15: biological and molecular effects. J |
| | Immunol. 2000 Oct 15; 165(8): 4312-8. |
| IL-2/IL-5 | B-cell proliferation/Ig secretion |
| | IL-5 induces IL-2 receptors on B-cells |
| IL-2/IL-6 | Development of cytotoxic T-cells |
| IL-2/IL-7 | |
| IL-2/IL-9 | See IL-2/IL-4 (NK-cells) |
| IL-2/IL-10 | B-cell activation |
| IL-2/IL-12 | IL-12 synergizes with IL-2 to induce lymphokine-activated |
| | cytotoxicity and perforin and granzyme gene expression in |
| | fresh human NK cells. Cell Immunol. 1995 Oct 1; 165(1): 33-43. |
| | (T-cell activation) |
| IL-2/IL-15 | See IL-2/IL-4 (NK cells) |
| | (T cell activation and proliferation) IL-15 and IL-2: a matter of |
| | life and death for T cells in vivo. Nat Med. 2001 Jan; 7(1): 114-8. |
| IL-2/IL-16 | Synergistic activation of CD4+ T cells by IL-16 and IL-2. J |
| | Immunol. 1998 Mar 1; 160(5): 2115-20. |
| IL-2/IL-17 | Evidence for the early involvement of interleukin 17 in human |
| | and experimental renal allograft rejection. J Pathol. 2002 |
| | Jul; 197(3): 322-32. |
| IL-2/IL-18 | Interleukin 18 (IL-18) in synergy with IL-2 induces lethal lung |
| | injury in mice: a potential role for cytokines, chemokines, and |
| | natural killer cells in the pathogenesis of interstitial |
| | pneumonia. Blood. 2002 Feb 15; 99(4): 1289-98. |
| IL-2/TGF-β | Control of CD4 effector fate: transforming growth factor beta |
| | 1 and interleukin 2 synergize to prevent apoptosis and promote |
| | effector expansion. J Exp Med. 1995 Sep 1; 182(3): 699-709. |
| IL-2/IFN-γ | Ig secretion by B-cells |
| | IL-2 induces IFN-γ expression by T-cells |
| IL-2/IFN-α/β | None |
| IL-3/IL-4 | Synergize in mast cell growth |
| | Synergistic effects of IL-4 and either GM-CSF or IL-3 on the |
| | induction of CD23 expression by human monocytes: |
| | regulatory effects of IFN-alpha and IFN-gamma. Cytokine. |
| | 1994 Jul; 6(4): 407-13. |
| IL-3/IL-5 | |
| IL-3/IL-6 | |
| IL-3/IFN-γ | IL-4 and IFN-gamma synergistically increase total polymeric |
| | IgA receptor levels in human intestinal epithelial cells. Role of |
| | protein tyrosine kinases. J Immunol. 1996 Jun |
| | 15; 156(12): 4807-14. |
| IL-3/GM-CSF | Differential regulation of human eosinophil IL-3, IL-5, and |
| | GM-CSF receptor alpha-chain expression by cytokines: IL-3, |
| | IL-5, and GM-CSF down-regulate IL-5 receptor alpha |
| | expression with loss of IL-5 responsiveness, but up-regulate |
| | IL-3 receptor alpha expression. J Immunol. 2003 Jun |
| | 1; 170(11): 5359-66. (allergic inflammation) |
| IL-4/IL-2 | IL-4 synergistically enhances both IL-2- and IL-12-induced |
| | IFN-{gamma} expression in murine NK cells. Blood. 2003 |
| | Mar 13 [Epub ahead of print] |
| IL-4/IL-5 | Enhanced mast cell histamine etc. secretion in response to IgE |
| | A Th2-like cytokine response is involved in bullous |
| | pemphigoid. the role of IL-4 and IL-5 in the pathogenesis of |
| | the disease. Int J Immunopathol Pharmacol. 1999 May-Aug; |
| | 12(2): 55-61. |
| IL-4/IL-6 | |
| IL-4/IL-10 | |
| IL-4/IL-11 | Synergistic interactions between interleukin-11 and |
| | interleukin-4 in support of proliferation of primitive |
| | hematopoietic progenitors of mice. Blood. 1991 Sep |
| | 15; 78(6): 1448-51. |
| IL-4/IL-12 | Synergistic effects of IL-4 and IL-18 on IL-12-dependent IFN- |
| | gamma production by dendritic cells. J Immunol. 2000 Jan |
| | 1; 164(1): 64-71. (increase Th1/Th2 differentiation) |
| | IL-4 synergistically enhances both IL-2- and IL-12-induced |
| | IFN-{gamma} expression in murine NK cells. Blood. 2003 |
| | Mar 13 [Epub ahead of print] |
| IL-4/IL-13 | Abstract Interleukin-4 and interleukin-13 signaling |
| | connections maps. Science. 2003 Jun 6; 300(5625): 1527-8. |
| | (allergy, asthma) |
| | Inhibition of the IL-4/IL-13 receptor system prevents allergic |
| | sensitization without affecting established allergy in a mouse |
| | model for allergic asthma. J Allergy Clin Immunol. 2003 |
| | Jun; 111(6): 1361-1369. |
| IL-4/IL-16 | (asthma) Interleukin (IL)-4/IL-9 and exogenous IL-16 induce |
| | IL-16 production by BEAS-2B cells, a bronchial epithelial cell |
| | line. Cell Immunol. 2001 Feb 1; 207(2): 75-80 |
| IL-4/IL-17 | Interleukin (IL)-4 and IL-17 synergistically stimulate IL-6 |
| | secretion in human colonic myofibroblasts. Int J Mol Med. |
| | 2002 Nov; 10(5): 631-4. (Gut inflammation) |
| IL-4/IL-24 | IL-24 is expressed by rat and human macrophages. |
| | Immunobiology. 2002 Jul; 205(3): 321-34. |
| IL-4/IL-25 | Abstract New IL-17 family members promote Th1 or Th2 |
| | responses in the lung: in vivo function of the novel cytokine |
| | IL-25. J Immunol. 2002 Jul 1; 169(1): 443-53. (allergic |
| | inflammation) |
| | Abstract Mast cells produce interleukin-25 upon Fc-epsilon |
| | RI-mediated activation. Blood. 2003 May 1; 101(9): 3594-6. |
| | Epub 2003 Jan 02. (allergic inflammation) |
| IL-4/IFN-γ | Abstract Interleukin 4 induces interleukin 6 production by |
| | endothelial cells: synergy with interferon-gamma. Eur J |
| | Immunol. 1991 Jan; 21(1): 97-101. |
| IL-4/SCF | Regulation of human intestinal mast cells by stem cell factor |
| | and IL-4. Immunol Rev. 2001 Feb; 179: 57-60. Review. |
| IL-5/IL-3 | Differential regulation of human eosinophil IL-3, IL-5, and |
| | GM-CSF receptor alpha-chain expression by cytokines: IL-3, |
| | IL-5, and GM-CSF down-regulate IL-5 receptor alpha |
| | expression with loss of IL-5 responsiveness, but up-regulate |
| | IL-3 receptor alpha expression. J Immunol. 2003 Jun |
| | 1; 170(11): 5359-66. (Allergic inflammation see abstract) |
| IL-5/IL-6 | |
| IL-5/IL-13 | Inhibition of allergic airways inflammation and airway hyper |
| | responsiveness in mice by dexamethasone: role of eosinophils. |
| | IL-5, eotaxin, and IL-13. J Allergy Clin Immunol. 2003 |
| | May; 111(5): 1049-61. |
| IL-5/IL-17 | Interleukin-17 orchestrates the granulocyte influx into airways |
| | after allergen inhalation in a mouse model of allergic asthma. |
| | Am J Respir Cell Mol Biol. 2003 Jan; 28(1): 42-50. |
| IL-5/IL-25 | Abstract New IL-17 family members promote Th1 or Th2 |
| | responses in the lung: in vivo function of the novel cytokine |
| | IL-25. J Immunol. 2002 Jul 1; 169(1): 443-53. (allergic |
| | inflammation) |
| | Abstract Mast cells produce interleukin-25 upon Fc-epsilon |
| | RI-mediated activation. Blood. 2003 May 1; 101(9): 3594-6. |
| | Epub 2003 Jan 02. (allergic inflammation) |
| IL-5/IFN-γ | |
| IL-5/GM-CSF | Differential regulation of human eosinophil IL-3, IL-5, and |
| | GM-CSF receptor alpha-chain expression by cytokines: IL-3, |
| | IL-5, and GM-CSF down-regulate IL-5 receptor alpha |
| | expression with loss of IL-5 responsiveness, but up-regulate |
| | IL-3 receptor alpha expression. J Immunol. 2003 Jun |
| | 1; 170(11): 5359-66. (Allergic inflammation) |
| IL-6/IL-10 | |
| IL-6/IL-11 | |
| IL-6/IL-16 | Interleukin-16 stimulates the expression and production of pro- |
| | inflammatory cytokines by human monocytes. Immunology. |
| | 2000 May; 100(1): 63-9. |
| IL-6/IL-17 | Stimulation of airway mucin gene expression by interleukin |
| | (IL)-17 through IL-6 paracrine/autocrine loop. J Biol Chem. |
| | 2003 May 9; 278(19): 17036-43. Epub 2003 Mar 06. (airway |
| | inflammation, asthma) |
| IL-6/IL-19 | Abstract IL-19 induces production of IL-6 and TNF-alpha and |
| | results in cell apoptosis through TNF-alpha. J Immunol. 2002 |
| | Oct 15; 169(8): 4288-97. |
| IL-6/IFN-g | |
| IL-7/IL-2 | Interleukin 7 worsens graft-versus-host disease. Blood. 2002 |
| | Oct 1; 100(7): 2642-9. |
| IL-7/IL-12 | Synergistic effects of IL-7 and IL-12 on human T cell |
| | activation. J Immunol. 1995 May 15; 154(10): 5093-102. |
| IL-7/IL-15 | Interleukin-7 and interleukin-15 regulate the expression of the |
| | bcl-2 and c-myb genes in cutaneous T-cell lymphoma cells. |
| | Blood. 2001 Nov 1; 98(9): 2778-83. (growth factor) |
| IL-8/IL-11 | Abnormal production of interleukin (IL)-11 and IL-8 in |
| | polycythaemia vera. Cytokine. 2002 Nov 21; 20(4): 178-83. |
| IL-8/IL-17 | The Role of IL-17 in Joint Destruction. Drug News Perspect. |
| | 2002 Jan; 15(1): 17-23. (arthritis) |
| | Abstract Interleukin-17 stimulates the expression of |
| | interleukin-8, growth-related oncogene-alpha, and |
| | granulocyte-colony-stimulating factor by human airway |
| | epithelial cells. Am J Respir Cell Mol Biol. 2002 |
| | Jun; 26(6): 748-53. (airway inflammation) |
| IL-8/GSF | Interleukin-8: an autocrine/paracrine growth factor for human |
| | hematopoietic progenitors acting in synergy with colony |
| | stimulating factor-1 to promote monocyte-macrophage growth |
| | and differentiation. Exp Hematol. 1999 Jan; 27(1): 28-36. |
| IL-8/VGEF | Intracavitary VEGF, bFGF, IL-8, IL-12 levels in primary and |
| | recurrent malignant glioma. J Neurooncol. 2003 |
| | May; 62(3): 297-303. |
| IL-9/IL-4 | Anti-interleukin-9 antibody treatment inhibits airway |
| | inflammation and hyperreactivity in mouse asthma model. Am |
| | J Respir Crit Care Med. 2002 Aug 1; 166(3): 409-16. |
| IL-9/IL-5 | Pulmonary overexpression of IL-9 induces Th2 cytokine |
| | expression, leading to immune pathology. J Clin Invest. 2002 |
| | Jan; 109(1): 29-39. |
| | Th2 cytokines and asthma. Interleukin-9 as a therapeutic target |
| | for asthma. Respir Res. 2001; 2(2): 80-4. Epub 2001 Feb 15. |
| | Review. |
| | Abstract Interleukin-9 enhances interleukin-5 receptor |
| | expression, differentiation, and survival of human eosinophils. |
| | Blood. 2000 Sep 15; 96(6): 2163-71 (asthma) |
| IL-9/IL-13 | Anti-interleukin-9 antibody treatment inhibits airway |
| | inflammation and hyperreactivity in mouse asthma model. Am |
| | J Respir Crit Care Med. 2002 Aug 1; 166(3): 409-16. |
| | Direct effects of interleukin-13 on epithelial cells cause airway |
| | hyperreactivity and mucus overproduction in asthma. Nat Med. |
| | 2002 Aug; 8(8): 885-9. |
| IL-9/IL-16 | See IL-4/IL-16 |
| IL-10/IL-2 | The interplay of interleukin-10 (IL-10) and interleukin-2 (IL- |
| | 2) in humoral immune responses: IL-10 synergizes with IL-2 |
| | to enhance responses of human B lymphocytes in a mechanism |
| | which is different from upregulation of CD25 expression. Cell |
| | Immunol. 1994 Sep; 157(2): 478-88. |
| IL-10/IL-12 | |
| IL-10/TGF-β | IL-10 and TGF-beta cooperate in the regulatory T cell |
| | response to mucosal allergens in normal immunity and specific |
| | immunotherapy. Eur J Immunol. 2003 May; 33(5): 1205-14. |
| IL-10/IFN-γ | |
| IL-11/IL-6 | Interleukin-6 and interleukin-11 support human osteoclast |
| | formation by a RANKL-independent mechanism. Bone. 2003 |
| | Jan; 32(1): 1-7. (bone resorption in inflammation) |
| IL-11/IL-17 | Polarized in vivo expression of IL-11 and IL-17 between acute |
| | and chronic skin lesions. J Allergy Clin Immunol. 2003 |
| | Apr; 111(4): 875-81. (allergic dermatitis) |
| | IL-17 promotes bone erosion in murine collagen-induced |
| | arthritis through loss of the receptor activator of NF-kappa B |
| | ligand/osteoprotegerin balance. J Immunol. 2003 Mar |
| | 1; 170(5): 2655-62. |
| IL-11/TGF-β | Polarized in vivo expression of IL-11 and IL-17 between acute |
| | and chronic skin lesions. J Allergy Clin Immunol. 2003 |
| | Apr; 111(4): 875-81. (allergic dermatitis) |
| IL-12/IL-13 | Relationship of Interleukin-12 and Interleukin-13 imbalance |
| | with class-specific rheumatoid factors and anticardiolipin |
| | antibodies in systemic lupus erythematosus. Clin Rheumatol. |
| | 2003 May; 22(2): 107-11. |
| IL-12/IL-17 | Upregulation of interleukin-12 and -17 in active inflammatory |
| | bowel disease. Scand J Gastroenterol. 2003 Feb; 38(2): 180-5. |
| IL-12/IL-18 | Synergistic proliferation and activation of natural killer cells |
| | by interleukin 12 and interleukin 18. Cytokine. 1999 |
| | Nov; 11(11): 822-30. |
| | Inflammatory Liver Steatosis caused by IL-12 and IL-18. J |
| | Interferon Cytokine Res. 2003 Mar; 23(3): 155-62. |
| IL-12/IL-23 | nterleukin-23 rather than interleukin-12 is the critical cytokine |
| | for autoimmune inflammation of the brain. Nature. 2003 Feb |
| | 13; 421(6924): 744-8. |
| | Abstract A unique role for IL-23 in promoting cellular |
| | immunity. J Leukoc Biol. 2003 Jan; 73(1): 49-56. Review. |
| IL-12/IL-27 | Abstract IL-27, a heterodimeric cytokine composed of EBI3 |
| | and p28 protein, induces proliferation of naive CD4(+) T cells. |
| | Immunity. 2002 Jun; 16(6): 779-90. |
| IL-12/IFN-γ | IL-12 induces IFN-γ expression by B and T-cells as part of |
| | immune stimulation. |
| IL-13/IL-5 | See IL-5/IL-13 |
| IL-13/IL-25 | Abstract New IL-17 family members promote Th1 or Th2 |
| | responses in the lung: in vivo function of the novel cytokine |
| | IL-25. J Immunol. 2002 Jul 1; 169(1): 443-53. (allergic |
| | inflammation) |
| | Abstract Mast cells produce interleukin-25 upon Fc-epsion |
| | RI-mediated activation. Blood. 2003 May 1; 101(9): 3594-6. |
| | Epub 2003 Jan 02. (allergic inflammation) |
| IL-15/IL-13 | Differential expression of interleukins (IL)-13 and IL-15 in |
| | ectopic and eutopic endometrium of women with |
| | endometriosis and normal fertile women. Am J Reprod |
| | Immunol. 2003 Feb; 49(2): 75-83. |
| IL-15/IL-16 | IL-15 and IL-16 overexpression in cutaneous T-cell |
| | lymphomas: stage-dependent increase in mycosis fungoides |
| | progression. Exp Dermatol. 2000 Aug; 9(4): 248-51. |
| IL-15/IL-17 | Abstract IL-17, produced by lymphocytes and neutrophils, is |
| | necessary for lipopolysaccharide-induced airway neutrophilia: |
| | IL-15 as a possible trigger. J Immunol. 2003 Feb |
| | 15; 170(4): 2106-12. (airway inflammation) |
| IL-15/IL-21 | IL-21 in Synergy with IL-15 or IL-18 Enhances IFN-gamma |
| | Production in Human NK and T Cells. J Immunol. 2003 Jun |
| | 1; 170(11): 5464-9. |
| IL-17/IL-23 | Interleukin-23 promotes a distinct CD4 T cell activation state |
| | characterized by the production of interleukin-17. J Biol |
| | Chem. 2003 Jan 17; 278(3): 1910-4. Epub 2002 Nov 03 |
| IL-17/TGF-β | Polarized in vivo expression of IL-11 and IL-17 between acute |
| | and chronic skin lesions. J Allergy Clin Immunol. 2003 |
| | Apr; 111(4): 875-81. (allergic dermatitis) |
| IL-18/IL-12 | Synergistic proliferation and activation of natural killer cells |
| | by interleukin 12 and interleukin 18. Cytokine. 1999 |
| | Nov; 11(11): 822-30. |
| | Abstract Inhibition of in vitro immunoglobulin production by |
| | IL-12 in murine chronic graft-vs.-host disease: synergism with |
| | IL-18. Eur J Immunol. 1998 Jun; 28(6): 2017-24. |
| IL-18/IL-21 | IL-21 in Synergy with IL-15 or IL-18 Enhances IFN-gamma |
| | Production in Human NK and T Cells. J Immunol. 2003 Jun |
| | 1; 170(11): 5464-9. |
| IL-18/TGF-β | Interleukin 18 and transforming growth factor betal in the |
| | serum of patients with Graves' ophthalmopathy treated with |
| | corticosteroids. Int Immunopharmacol. 2003 Apr; 3(4): 549-52. |
| IL-18/IFN-γ | |
| Anti-TNF | Synergistic therapeutic effect in DBA/1 arthritic mice. |
| ALPHA/anti- | |
| CD4 | |

| Target | Disease | Pair with |
|---|---|---|
| CD89* | Use as cytotoxic cell | all |
| | recruiter | |
| CD19 | B cell lymphomas | HLA-DR |
| | | CD5 |
| HLA-DR | B cell lymphomas | CD89 |
| | | CD19 |
| | | CD5 |
| CD38 | Multiple myeloma | CD138 |
| | | CD56 |
| | | HLA-DR |
| CD138 | Multiple myeloma | CD38 |
| | | CD56 |
| | | HLA-DR |
| CD138 | Lung cancer | CD56 |
| | | CEA |
| CD33 | Acute myelod lymphoma | CD34 |
| | | HLA-DR |
| CD56 | Lung cancer | CD138 |
| | | CEA |
| CEA | Pan carcinoma | MET receptor |
| VEGF | Pan carcinoma | MET receptor |
| VEGF | Pan carcinoma | MET receptor |
| receptor | | |
| IL-13 | Asthma/pulmonary | IL-4 |
| | inflammation | IL-5 |
| | | Eotaxin(s) |
| | | MDC |
| | | TARC |
| | | TNFα |
| | | IL-9 |
| | | EGFR |
| | | CD40L |
| | | IL-25 |
| | | MCP-1 |
| | | TGFβ |
| IL-4 | Asthma | IL-13 |
| | | IL-5 |
| | | Eotaxin(s) |
| | | MDC |
| | | TARC |
| | | TNFα |
| | | IL-9 |
| | | EGFR |
| | | CD40L |
| | | IL-25 |
| | | MCP-1 |
| | | TGFβ |
| Eotaxin | Asthma | IL-5 |
| | | Eotaxin-2 |
| | | Eotaxin-3 |
| EGFR | cancer | HER2/neu |
| | | HER3 |
| | | HER4 |
| HER2 | cancer | HER3 |
| | | HER4 |
| TNFR1 | RA/Crohn's disease | IL-1R |
| | | IL-6R |
| | | IL-18R |
| TNFα | RA/Crohn's disease | IL-1α/β |
| | | IL-6 |
| | | IL-18 |
| | | ICAM-1 |
| | | IL-15 |
| | | IL-17 |
| IL-1R | RA/Crohn's disease | IL-6R |
| | | IL-18R |
| IL-18R | RA/Crohn's disease | IL-6R |

### EpCAM

### CD20

### CD33

### CD52

### Her-2/neu

### GPIIb/IIIa

### RSV

### CD25

### CD3

a4B3

e) A human version of a target ligand in a) to d).

### Annex 2

### Examples of Generic Ligands

a) Published single variable domains
Any variable domain disclosed in WO030020609, WO2004101790, WO2005035572, WO2004081026, WO2004003019 and WO2004058821, the disclosure of these variable domains, their sequences and method of production and selection being explicitly incorporated by reference herein to provide the skilled addressee with examples of generic ligands for use in the present invention.
Any VHH domain or any other variable domain disclosed in WO9404678, WO9748905, WO9933221, WO9937681, WO0024884, WO0043507, WO0065057, WO0140310, WO03035694, WO03053531, WO03054015, WO0305527, WO2004015425, WO2004041862, WO2004041863, WO20040401865, WO2004062551, WO2005044858 and EP1134231 the disclosure of these variable domains, their sequences and method of production and selection being explicitly incorporated by reference herein to provide the skilled addressee with examples of generic ligands for use in the present invention.

b)
(i) a human VH derived from germline VH segment of the 3-23 locus or any other locus in FIG. 7a).
(ii) a human VH having a FW1 amino acid sequence that is the same as the amino acid sequence of the corresponding FW from germline VH segment of the 3-23 locus or any other locus in FIG. 7a), or has up to 5 amino acid differences from said corresponding FW.
(iii) a human VH having a FW2 amino acid sequence that is the same as the amino acid sequence of the corresponding FW from germline VH segment of the 3-23 locus or any other locus in FIG. 7a), or has up to 5 amino acid differences from said corresponding FW.
(iv) a human VH having a FW3 amino acid sequence that is the same as the amino acid sequence of the corresponding FW from germline VH segment of the 3-23 locus or any other locus in FIG. 7a), or has up to 5 amino acid differences from said corresponding FW.
(v) a human VH having a FW4 amino acid sequence that is the same as the amino acid sequence of the corresponding FW from germline VH segment of the 3-23 locus or any other locus in FIG. 7a), or has up to 5 amino acid differences from said corresponding FW.
(vi) a human VH having FW 1, 2, 3 and 4 amino acid sequences that are the same as the amino acid sequences of the corresponding FWs from germline VH segment of the 3-23 locus or any other locus in FIG. 7a), or collectively where the FW 1, 2, 3 and 4 amino acid sequences have up to 10 amino acid differences from said corresponding FWs.
(vii) a human Vₖ derived from germline Vₖ segment of the DPK9 locus or any locus in FIG. 7b).
(viii) a human Vₖ having a FW1 amino acid sequence that is the same as the amino acid sequence of the corresponding FW from germline Vₖ segment of the DPK9 locus or any locus in FIG. 7b), or has up to 5 amino acid differences from said corresponding FW.
(ix) a human Vₖ having a FW2 amino acid sequence that is the same as the amino acid sequence of the corresponding FW from germline Vₖ segment of the DPK9 locus or any locus in FIG. 7b), or has up to 5 amino acid differences from said corresponding FW.
(x) a human Vₖ having a FW3 amino acid sequence that is the same as the amino acid sequence of the corresponding FW from germline Vₖ segment of the DPK9 locus or any locus in FIG. 7b), or has up to 5 amino acid differences from said corresponding FW.
(xi) a human Vₖ having a FW4 amino acid sequence that is the same as the amino acid sequence of the corresponding FW from germline Vₖ segment of the DPK9 locus or any locus in FIG. 7b), or has up to 5 amino acid differences from said corresponding FW.
(xii) a human VH having FW 1, 2, 3 and 4 amino acid sequences that are the same as the amino acid sequences of the corresponding FWs from germline Vₖ segment of the DPK9 locus or any locus in FIG. 7b), or collectively where the FW 1, 2, 3 and 4 amino acid sequences have up to 10 amino acid differences from said corresponding FWs.
(xiii) a human Vλ derived from any germline Vλ segment in FIG. 7c).
(xiv) a human Vλ having a FW1 amino acid sequence that is the same as the amino acid sequence of the corresponding FW from any germline Vλ segment in FIG. 7c), or has up to 5 amino acid differences from said corresponding FW.
(xv) a human Vλ having a FW2 amino acid sequence that is the same as the amino acid sequence of the corresponding FW from any germline Vλ segment in FIG. 7c), or has up to 5 amino acid differences from said corresponding FW.
(xvi) a human Vλ having a FW3 amino acid sequence that is the same as the amino acid sequence of the corresponding FW from any germline Vλ segment in FIG. 7c), or has up to 5 amino acid differences from said corresponding FW.
(xvii) a human Vλ having a FW4 amino acid sequence that is the same as the amino acid sequence of the corresponding FW from any germline Vλ segment in FIG. 7c), or has up to 5 amino acid differences from said corresponding FW.
(xviii) a human Vλ having FW 1, 2, 3 and 4 amino acid sequences that are the same as the amino acid sequences of the corresponding FWs from any germline Vλ segment in FIG. 7c), or collectively where the FW 1, 2, 3 and 4 amino acid sequences have up to 10 amino acid differences from said corresponding FWs.
(xix) a Camelid VHH or Nanobody™ derived from any germline segment in FIG. 7d) or disclosed in FIG. 2 (page 924) of Nguyen et al, EMBO J, 2000, Vol 19, No. 5, pp 921-930 (the disclosure of which, in particular the VHH germline sequences disclosed therein, are incorporated by reference).
(xx) a Camelid VHH or Nanobody™ having a FW1 amino acid sequence that is the same as the amino acid sequence of the corresponding FW from any germline VHH segment disclosed in FIG. 2 (page 924) of Nguyen et al, EMBO J, 2000, Vol 19, No. 5, pp 921-930, or has up to 5 amino acid differences from said corresponding FW.
(xxi) a Camelid VHH or Nanobody™ having a FW2 amino acid sequence that is the same as the amino acid sequence of the corresponding FW from any VHH germline segment disclosed in FIG. 2 (page 924) of Nguyen et al, EMBO J, 2000, Vol 19, No. 5, pp 921-930, or has up to 5 amino acid differences from said corresponding FW.
(xxii) a Camelid VHH or Nanobody™ having a FW3 amino acid sequence that is the same as the amino acid sequence of the corresponding FW from any VHH germline segment disclosed in FIG. 2 (page 924) of Nguyen et al, EMBO J, 2000, Vol 19, No. 5, pp 921-930, or has up to 5 amino acid differences from said corresponding FW.
(xxiii) a Camelid VHH or Nanobody™ having a FW4 amino acid sequence that is the same as the amino acid sequence of the corresponding FW from any VHH germline segment disclosed in FIG. 2 (page 924) of Nguyen et al, EMBO J, 2000, Vol 19, No. 5, pp 921-930, or has up to 5 amino acid differences from said corresponding FW.
(xxiv) a Camelid VHH or Nanobody™ having FW 1, 2, 3 and 4 amino acid sequences that are the same as the amino acid sequences of the corresponding FWs from any VHH germline segment disclosed in FIG. 2 (page 924) of Nguyen et al, EMBO J, 2000, Vol 19, No. 5, pp 921-930, or collectively where the FW 1, 2, 3 and 4 amino acid sequences have up to 10 amino acid differences from said corresponding FWs.

c)
(i) VH or VL from any of the Following Antibody Products:

| Target | Product | Ab Type | Condition |
|---|---|---|---|
| EpCAM | Panorex | murine | Colon cancer |
| CD20 | Rituxin | Chimeric IgG1 | Non-Hodgkin's |
| | | | lymhoma (NHL) |
| CD20 | Zevalin | murine 90Y | NHL |
| | | humanized IgG4 | AML (acute |
| | | toxin drug | myeloid |
| CD33 | Mylotarg | conjugate | leukaemia) |
| CD52 | Campath-1H | Humanized IgG1 | B-CLL |
| Her-2/neu | Herceptin | Humanized IgG1 | Breast cancer |
| GPIIb/IIIa | ReoPro | chimeric Fab | Angina |
| RSV | Synagis | Humanized IgG1 | Respiratory |
| | | | syncitial virus |
| | | | (RSV) |
| IgE | Xolair | humanized IgG1 | asthma, allergic |
| | | | rhinitis |
| TNF-alpha | Remicade | chimeric IgG1 | Rheumatoid |
| | | | arthritis (RA), |
| | | | Crohn's disease, |
| | | | psoriasis, |
| | | | ankylosing |
| | | | spondylitis, |
| | | | psoriatic arthritis, |
| | | | ulcerative colitis |
| CD25 | Simulect | chimeric IgG1 | Transplant |
| | | | rejection |
| CD3 | OKT3, | murine IgG2a | Transplant |
| | Orthoclone | | rejection |
| CD25 | Zenapax | Humanized IgG1 | Kidney transplant |
| TNF-alpha | Humira | Human IgG1 | RA, Crohn's |
| | | | disease, psoriasis, |
| | | | ankylosing |
| | | | spondylitis, |
| | | | psoriatic arthritis, |
| | | | ulcerative colitis |
| CD20 | Bexxar | | NHL |
| CD11a | Raptiva | | Psoriasis |
| a4B3 | Antegren | | Multiple sclerosis |
| EGFR | Erbitux | | Colorectal cancer |
| VEGF | Avastin | | Colorectal cancer, |
| | | | renal cancer |

(ii) A variable domain (VH or VL) of an anti-TNF antibody or antibody fragment disclosed in U.S. Pat. No. 6,090,382, US20030235585A1, US20040166111A1, EP1500329A2, US20040131613A1, US20030144484A1, U.S. Pat. No. 5,698,195, EP1159003A1, US20020119152A1, EP0871641A4, EP0710121B1,U.S. Pat. No. 5,702,705, EP0487610B1, the disclosure of which (in particular disclosure of the generation, sequence and utility of the antibodies and fragments therein) are expressly incorporated by reference to provide the skilled addressee with such information for use in the present invention.
(iii) An antibody heavy or light chain variable domain (e.g. VH, VL or VHH) that dissociates from target ligand with a K_{d} of 1×10⁻⁸ M or less determined by surface plasmon resonance. In a preferred embodiment the target ligand is a target ligand described in Annex 1. For guidance of surface plasmon resonance, the skilled addressee is directed to U.S. Pat. No. 6,090,382 or WO04003019A2.
(iv) An antibody heavy or light chain variable domain (e.g. VH, VL or VHH) that dissociates from target ligand with a K_{off} rate constant of 1×10⁻³ s⁻¹ or less determined by surface plasmon resonance. In a preferred embodiment the target ligand is a target ligand described in Annex 1. For guidance of surface plasmon resonance, the skilled addressee is directed to U.S. Pat. No. 6,090,382 or WO04003019A2.
(v) An antibody heavy or light chain variable domain (e.g. VH, VL or VHH) that dissociates from target ligand with a K_{d} of 1×10⁻⁸ M or less and a K_{off} rate constant of 1×10⁻³ s⁻¹ or less determined by surface plasmon resonance. In a preferred embodiment the target ligand is a target ligand described in Annex 1. For guidance of surface plasmon resonance, the skilled addressee is directed to U.S. Pat. No. 6,090,382 or WO04003019A2.
(vi) An antibody heavy or light chain variable domain (e.g. VH, VL or VHH) that dissociates from target ligand with a K_{d} of 1×10⁻⁸ M or less and a K_{off} rate constant of 1×10⁻³ s⁻¹ or less, both determined by surface plasmon resonance, and neutralizes target ligand cytotoxicity in a standard assay with an IC₅₀ of 1×10⁻⁷ M or less. In a preferred embodiment the target ligand is a target ligand described in Annex 1. For guidance of surface plasmon resonance, the skilled addressee is directed to U.S. Pat. No. 6,090,382 or WO04003019A2. In one embodiment, the target ligand is TNF alpha and the standard assay is a L929 assay as described in U.S. Pat. No. 6,090,382.

In the embodiments above,

Preferably, the generic ligand binds with a K_{d} is 1×10⁻⁹ M or less, 1×10⁻¹⁰ M or less, 1×10⁻¹¹M or less, 1×10⁻¹² M or less.

Preferably, the K_{off} rate constant is 1×10⁻⁴ s⁻¹ or less, 1×10⁻⁵ s⁻¹ or less, 1×10⁻⁶ s⁻¹ or less, 1×10⁻⁷ s⁻¹ or less, 1×10⁻⁸ s⁻¹ or less.

Preferably, the IC₅₀ is 1×10⁻⁸ M or less, 1×10⁻⁹ M or less, 5×10⁻¹⁰ M or less, 1×10⁻¹⁰ M or less, 5×10⁻¹¹M or less.
d) an antibody variable domain having a sequence that is at least 90% homologous to a sequence in Annex 2 (a).
(e) an antibody variable domain having a sequence that is at least 90% homologous to a sequence in Annex 2 (c).

1. A method for selecting, from a repertoire of antibody polypeptides, a population of functional variable domains which bind a target ligand and a generic ligand, which generic ligand is capable of binding functional members of the repertoire regardless of target ligand specificity, comprising the steps of:
a) contacting the repertoire with said generic ligand and selecting functional variable domains bound thereto; and
b) contacting the selected functional variable domains with the target ligand and selecting a population of variable domains which bind to the target ligand,
wherein either (i) the variable domains are heavy chain variable domains and the generic ligand is an antibody light chain variable domain; or (ii) the variable domains are light chain variable domains and the generic ligand is an antibody heavy chain variable domain; and
wherein optionally in (i) the heavy chain variable domains are Camelid variable domains (VHH) or derived from a Camelid heavy chain antibody (H2 antibody);
or optionally in (i) and (ii) each variable domain is a human variable domain or derived from a human.

2. A method according to claim 1 wherein the repertoire of antibody polypeptides is first contacted with the target ligand and then with the generic ligand.

3. A method according to claim 1 wherein the generic ligand binds a subset of the repertoire of variable domains.

4. A method according to claim 3 wherein two or more subsets are selected from the repertoire of polypeptides.

5. A method according to claim 4 wherein the selection is performed with two or more generic ligands, optionally two or more light chain variable domains (for option (i)) or two or more heavy chain variable domains (for option (ii)).

6. A method according to claims 4 wherein the two or more subsets are combined after selection to produce a further repertoire of polypeptides.

7. A method according to claim 1 wherein two or more repertoires of polypeptides are contacted with generic ligands and the subsets of polypeptides thereby obtained are then combined.

8. (canceled)

9. A method wherein a population of antibody heavy chain variable domains is selected according to claim 1 and a population of antibody light chain variable domains is selected according to claim 1 and the populations thereby obtained are then combined.

10. (canceled)

11. A method for selecting at least one antibody heavy chain variable domain from a population of antibody polypeptides, the method comprising:
a) contacting the population with an antibody light chain variable domain and
b) selecting at least one antibody heavy chain variable domain that binds to the light chain variable domain.

12. The method of claim 11, comprising prior to step a), the step of contacting antibody polypeptides with a target ligand and selecting antibody polypeptides that bind the target ligand, thereby providing said population of antibody polypeptides used in step a).

13. The method of claim 11, comprising after to step b), the step of contacting antibody heavy chain variable domains selected in step b) with a target ligand and selecting heavy chain variable domains that bind the target ligand.

14. The method of claim 11, wherein each heavy chain domain selected in step b) is from the group consisting of heavy chain variable domains derived from a Camelid; a VHH domain; a Nanobody™; a VHH having a glycine at position 44; a VHH having a leucine at position 45; a VHH having a tryptophan at position 47; a VHH having a glycine at position 44 and a leucine at position 45; a VHH having a glycine at position 44 and a tryptophan at position 47; a VHH having a leucine at position 45 and a tryptophan at position 47; a VHH having a glycine at position 44, a leucine at position 45 and a tryptophan at position 47; a VHH having a tryptophan or arginine at position 103.

15. The method of claim 11, wherein each heavy chain domain selected in step b) is a humanised Camelid or murine heavy chain variable domain or a humanised Nanobody™.

16. The method of claim 11, wherein each heavy chain domain selected in step b) is a human heavy chain variable domain.

17. The method of claim 11, wherein the light chain variable domain is a human light chain variable domain or derived from a human or a light chain variable domain having a FW2 sequence that is identical to FW2 encoded by germline gene sequence DPK9.

18. The method of claim 11, wherein the light chain variable domain is a Camelid light chain variable domain or derived from a Camelid.

19-24. (canceled)

25. The method of claim 11, wherein the population used in step a) comprises (i) antibody polypeptides each comprising at least one heavy chain variable domain that is not paired with a light chain variable domain; and (ii) antibody polypeptides each comprising a heavy chain variable domain that is paired with a light chain variable domain.

26-34. (canceled)

35. An isolated antibody polypeptide comprising or consisting of an antibody heavy chain variable domain, wherein the polypeptide is obtainable by the method of claim 11, wherein the light chain variable domain in the method is a human light chain variable domain and the heavy chain variable domain is from a non-human mammal and wherein the heavy chain variable domain is a humanized heavy chain variable domain.

36. The antibody polypeptide of claim 35, wherein the heavy chain variable domain is from the group consisting of a heavy chain variable domain derived from a Camelid; a VHH domain; a Nanobody™; a VHH having a glycine at position 44; a VHH having a leucine at position 45; a VHH having a tryptophan at position 47; a VHH having a glycine at position 44 and a leucine at position 45; a VHH having a glycine at position 44 and a tryptophan at position 47; a VHH having a leucine at position 45 and a tryptophan at position 47; a VHH having a glycine at position 44, a leucine at position 45 and a tryptophan at position 47; a VHH having a tryptophan or arginine at position 103.

37. The antibody polypeptide of claim 36, wherein the heavy chain variable domain is provided as part of a Camelid IgG or an IgG derived from a Camelid.

38. The antibody polypeptide of claim 36, wherein the heavy chain variable domain is provided as part of a human IgG or an IgG derived from a human, and wherein the heavy chain variable domain is paired in the IgG with a light chain variable domain that is different from the light chain variable domain recited in claim 11.

39. (canceled)

40. The method of claim 11, wherein the heavy chain variable domain binds a target ligand selected from the group consisting of TNF alpha, serum albumin, von Willebrand's factor (vWF), IgE, interferon gamma, EGFR, IgE, MMP12, PDK1 and Amyloid beta (A-beta), or any one of the targets listed in Annex 1.

41. The antibody of claim 35, wherein the heavy chain variable domain binds a target ligand selected from the group consisting of TNF alpha, serum albumin, von Willebrand's factor (vWF), IgE, interferon gamma, EGFR, IgE, MMP12, PDK1 and Amyloid beta (A-beta), or any one of the targets listed in Annex 1.

42. A method for selecting at least one antibody light chain variable domain from a population of antibody polypeptides, the method comprising:
a) contacting the population with an antibody heavy chain variable domain and
b) selecting at least one antibody light chain variable domain that binds to the heavy chain variable domain.

43. The method of claim 42, comprising prior to step a), the step of contacting antibody polypeptides with a target ligand and selecting antibody polypeptides that bind the target ligand, thereby providing said population of antibody polypeptides used in step a).

44. The method of claim 42, comprising after to step b), the step of contacting antibody light chain variable domains selected in step b) with a target ligand and selecting light chain variable domains that bind the target ligand.

45. The method of claim 42, wherein each light chain domain selected in step b) is derived from a Camelid.

46. The method of claim 42, wherein each light chain domain selected in step b) is a human light chain variable domain, or a humanized light chain variable domain, optionally a humanised camelid or murine variable domain.

47. The method of claim 42, wherein the heavy chain variable domain is a human heavy chain variable domain; derived from a human; a heavy chain variable domain having a FW2 sequence that is identical to FW2 encoded by germline gene sequence DP47; or a heavy chain variable domain having positions 44, 45 and 47 that are identical to positions 44, 45 and 47 encoded by germline gene sequence DP47.

48. The method of claim 42, wherein the heavy chain variable domain is a Camelid heavy chain variable domain (VHH or VH) or derived from a Camelid.

49. The method of claim 42, wherein the population in step a) is provided by a population of B-cells.

50. The method of claim 49, wherein the B-cells are peripheral blood lymphocytes.

51. The method of claim 49, wherein the B-cells are isolated from an animal that has been immunised with a target antigen.

52. The method of claim 49, wherein the B-cells are isolated from an animal that has not been immunised with a target antigen.

53. The method of claim 42, wherein the population used in step a) is provided by a repertoire of antibody polypeptides encoded by synthetically rearranged antibody genes.

54. The method of claim 42, wherein the population used in step a) is provided by a phage display library comprising bacteriophage displaying said antibody polypeptides.

55. The method of claim 42, wherein the population used in step a) comprises (i) antibody polypeptides each comprising at least one light chain variable domain that is not paired with a heavy chain variable domain; and (ii) antibody polypeptides each comprising a light chain variable domain that is paired with a heavy chain variable domain.

56. The method of claim 42, wherein the population used in step a) comprises human light chain single variable domains (VL).

57-60. (canceled)

61. An isolated antibody polypeptide comprising or consisting of an antibody light chain variable domain, wherein the polypeptide is obtainable by the method of claim 42, wherein the heavy chain variable domain in the method is a human heavy chain variable domain and the light chain variable domain is from a non-human mammal.

62. The antibody polypeptide of claim 61, wherein the light chain variable domain is from a Camelid.

63. The antibody polypeptide of claim 61, wherein the light chain variable domain is provided as part of a Camelid IgG or an IgG derived from a Camelid.

64. The antibody polypeptide of claim 61, wherein the light chain variable domain is provided as part of a human IgG or an IgG derived from a human, and wherein the light chain variable domain is paired in the IgG with a heavy chain variable domain that is different from the heavy chain variable domain recited in claim 42.

65-66. (canceled)

67. A polypeptide comprising a half-life extending moiety linked to an antibody polypeptide of claim 35, wherein the moiety is selected from a PEG; an antibody constant domain; an antibody Fc region; albumin or a fragment thereof; a peptide or an antibody fragment that binds albumin, an albumin fragment; the neonatal Fc receptor; transferring; or the transferrin receptor.

68. (canceled)

69. The method of claim 42, wherein the light chain variable domain or the variable domain of the antibody polypeptide binds a target ligand selected from the group consisting of TNF alpha, serum albumin, von Willebrand's factor (vWF), IgE, interferon gamma, EGFR, IgE, MMP12, PDK1 and Amyloid beta (A-beta), or any one of the targets listed in Annex 1.

70. The antibody polypeptide of claim 61, wherein the light chain variable domain or the variable domain of the antibody polypeptide binds a target ligand selected from the group consisting of TNF alpha, serum albumin, von Willebrand's factor (vWF), IgE, interferon gamma, EGFR, IgE, MMP12, PDK1 and Amyloid beta (A-beta), or any one of the targets listed in Annex 1.

71. The method of claim 1, comprising the step of producing a mutant or derivative of the selected variable domain.

72-73. (canceled)

74. A method for separating IgG from Camelid VNH antibody single variable domain in a population of antibody polypeptides comprising single variable domains and IgG, the method comprising:
a) contacting the population with a generic ligand and
b) selecting a subpopulation that binds to the generic ligand, thereby separating IgG from the single variable domain,
wherein the generic ligand has binding specificity for antibody CH1 domain, light chain constant domain (CL), IgG hinge or antibody light chain variable domain.

75-80. (canceled)

81. A method for separating a Camelid VHH single variable domain from IgG in a population of antibody polypeptides comprising Camelid VHH domains and IgG, the method comprising:
a) contacting the population with a generic ligand and
b) selecting a subpopulation that binds to the generic ligand, thereby separating the single variable domain from IgG,
wherein the generic ligand has binding specificity for (i) VHH and not VH; or (ii) heavy chain antibody (H2) hinge.

82-91. (canceled)

92. A method for selecting, from a repertoire of antibody polypeptides, a single variable domain which binds a target ligand and a generic ligand, comprising the steps of:
a) contacting the repertoire with a target ligand and selecting single variable domains bound thereto; and
b) contacting the selected variable domains with the generic ligand and selecting a variable domain which binds to the generic ligand,
wherein the generic ligand is an antibody variable domain selected from Annex 2 c) or e); and
wherein (i) when the selected variable domain is a heavy chain variable domain the generic ligand is a light chain variable domain, or (ii) when the selected variable domain is a light chain variable domain the generic ligand is a heavy chain variable domain.

93-94. (canceled)

95. The method of claim 92, comprising the step of producing a mutant or derivative of the selected variable domain.

96. The method of claim 92, wherein the generic ligand binds the same target ligand species as the selected variable domain.

97. The method of claim 92, wherein the generic ligand binds a different target ligand species to the selected variable domain.

98. The method of claim 92, comprising combining the selected variable domain with an antibody variable domain that is identical to the generic ligand or a derivative thereof to produce a product with target ligand binding specificity.

99. A method of producing a derivative of an antibody or antibody fragment in any of Annex 2c) (i) to (iv) that binds a target ligand, the method comprising:
a) using a heavy chain variable domain of said antibody or fragment or an identical variable domain as the generic ligand in the method of claim 92, and wherein the target ligand used in step a) is the target ligand to which the antibody or fragment binds, thereby selecting a light chain single variable domain that binds the target ligand and the heavy chain variable domain; and
b) replacing at least one of the light chain variable domains of the antibody or fragment with the selected light chain variable domain; an identical light chain variable domain or a derivative thereof.

100. A method of producing multispecific derivative of an antibody or antibody fragment in any of Annex 2c) (i) to (iv), the method comprising:
a) using a heavy chain variable domain of said antibody or fragment or an identical variable domain as the generic ligand in the method of claim 92, and wherein the target ligand used in step a) is a target ligand that is different from the target ligand to which the antibody or fragment binds, thereby selecting a light chain single variable domain that binds the different target ligand and the heavy chain variable domain; and
b) replacing at least one of the light chain variable domains of the antibody or fragment with the selected light chain variable domain; an identical light chain variable domain or a derivative thereof, thereby producing a multispecific product.

101. A method of producing a derivative of an antibody or antibody fragment in any of Annex 2c) (i) to (iv) that binds a target ligand, the method comprising:
a) using a light chain variable domain of said antibody or fragment or an identical variable domain as the generic ligand in the method of claim, and wherein the target ligand used in step a) is the target ligand to which the antibody or fragment binds, thereby selecting a heavy chain single variable domain that binds the target ligand and the light chain variable domain; and
b) replacing at least one of the heavy chain variable domains of the antibody or fragment with the selected heavy chain variable domain; an identical heavy chain variable domain or a derivative thereof.
c)

102. A method of producing multispecific derivative of an antibody or antibody fragment in any of Annex 2c) (i) to (iv), the method comprising:
a) using a light chain variable domain of said antibody or fragment or an identical variable domain as the generic ligand in the method of claim 92, and wherein the target ligand used in step a) is a target ligand that is different from the target ligand to which the antibody or fragment binds, thereby selecting a heavy chain single variable domain that binds the different target ligand and the light chain variable domain; and
b) replacing at least one of the heavy chain variable domains of the antibody or fragment with the selected heavy chain variable domain;
an identical heavy chain variable domain or a derivative thereof, thereby producing a multispecific product.

103. The method of claim 100, wherein the heavy chain variable domain is selected from is from the group consisting of heavy chain variable domains derived from a Camelid; a VHH domain; a Nanobody™; a VHH having a glycine at position 44; a VHH having a leucine at position 45; a VHH having a tryptophan at position 47; a VHH having a glycine at position 44 and a leucine at position 45; a VHH having a glycine at position 44 and a tryptophan at position 47; a VHH having a leucine at position 45 and a tryptophan at position 47; a VHH having a glycine at position 44, a leucine at position 45 and a tryptophan at position 47; a VHH having a tryptophan or arginine at position 103; a humanised Camelid or murine heavy chain variable domain; a humanised Nanobody™; a human heavy chain variable domain; a heavy chain variable domain derived from a human; a heavy chain variable domain having a FW2 sequence that is identical to FW2 encoded by germline gene sequence DP47; or a heavy chain variable domain having positions 44, 45 and 47 that are identical to positions 44, 45 and 47 encoded by germline gene sequence DP47.

104. The method of claim 99, wherein the light chain variable domain is selected from a human light chain variable domain; a light chain variable domain derived from a human; a light chain variable domain having a FW2 sequence that is identical to FW2 encoded by germline gene sequence DPK9; a Camelid light chain variable domain; a light chain variable domain derived from a Camelid; and a humanised Camelid or murine light chain variable domain.

105. The method of claim 99, wherein either a) the selected variable domain is a heavy chain variable domain and each heavy chain variable domain of the antibody or fragment is replaced with the selected heavy chain variable domain; an identical heavy chain variable domain or a derivative thereof; or b) the selected variable domain is a light chain variable domain and each light chain variable domain of the antibody or fragment is replaced with the selected light chain variable domain; an identical light chain variable domain or a derivative thereof.

106-107. (canceled)

108. The method of claim 100, wherein the wherein the generic ligand is an antibody variable domain from an antibody or antibody fragment selected from Panorex™, Rituxin™, Zevalin™, Mylotarg™, Campath™, Herceptin™, ReoPro™, Synagis™, Xolair™, Remicade™, Simulect™, OKT3™, Orthoclone™, Zenapax™, Humira™, Bexxar™, Raptiva™, Antegren™, Erbitux™ and Avastin™ or an identical variable domain or a derivative thereof that binds the target ligand bound by the antibody or antibody fragment.

109-112. (canceled)

113. The antibody polypeptide of claim 35, wherein the heavy chain variable domain in a humanized camelid or murine heavy chain variable domain.

114. The antibody polypeptide of claim 61, wherein the higher chain variable domain is a humanized camelid or murine light chain variable domain.
